SEQUENCE LISTING (106 sequences)

SEQ ID NO: 1 - DNA, 20 bp, synthetic construct
  source 1..20: mol_type = other DNA
cgctctctgctcctcctgtt

SEQ ID NO: 2 - DNA, 19 bp, synthetic construct
  source 1..19: mol_type = other DNA
gcaaccacgaacggacatc

SEQ ID NO: 3 - DNA, 20 bp, synthetic construct
  source 1..20: mol_type = other DNA
cggataccttccctagtgcc

SEQ ID NO: 4 - DNA, 20 bp, synthetic construct
  source 1..20: mol_type = other DNA
cagaatgcacgctacaggaa

SEQ ID NO: 5 - DNA, 20 bp, synthetic construct
  source 1..20: mol_type = other DNA
gcaccaaggatggagatgtt

SEQ ID NO: 6 - DNA, 21 bp, synthetic construct
  source 1..21: mol_type = other DNA
ggatgagcctacaactggctt

SEQ ID NO: 7 - DNA, 19 bp, synthetic construct
  source 1..19: mol_type = other DNA
gtggtgggaactttggctg

SEQ ID NO: 8 - DNA, 20 bp, synthetic construct
  source 1..20: mol_type = other DNA
atcgagacgttaccccacaa

SEQ ID NO: 9 - DNA, 21 bp, synthetic construct
  source 1..21: mol_type = other DNA
gtgtttctggtcagcccaact

SEQ ID NO: 10 - DNA, 18 bp, synthetic construct
  source 1..18: mol_type = other DNA
tccaactgtgcttcaagc

SEQ ID NO: 11 - DNA, 21 bp, synthetic construct
  source 1..21: mol_type = other DNA
gatgatgcgggagaagaaggt

SEQ ID NO: 12 - DNA, 21 bp, synthetic construct
  source 1..21: mol_type = other DNA
cttcccctccgctgctcacta

SEQ ID NO: 13 - DNA, 24 bp, synthetic construct
  source 1..24: mol_type = other DNA
tgcatcttgctgaagtatgtcacc

SEQ ID NO: 14 - DNA, 19 bp, synthetic construct
  source 1..19: mol_type = other DNA
tctcggtctggaggatgga

SEQ ID NO: 15 - DNA, 19 bp, synthetic construct
  source 1..19: mol_type = other DNA
ggctgttcccttgcttcct

SEQ ID NO: 16 - DNA, 22 bp, synthetic construct
  source 1..22: mol_type = other DNA
ggatgactctgacaacttctcc

SEQ ID NO: 17 - DNA, 25 bp, synthetic construct
  source 1..25: mol_type = other DNA
cagtgtggtatctctacaggatgga

SEQ ID NO: 18 - DNA, 20 bp, synthetic construct
  source 1..20: mol_type = other DNA
caagtctgcagtggtgtttg

SEQ ID NO: 19 - DNA, 20 bp, synthetic construct
  source 1..20: mol_type = other DNA
gccgagagctacacgttcac

SEQ ID NO: 20 - DNA, 22 bp, synthetic construct
  source 1..22: mol_type = other DNA
caacatacagtgacgcttcaca

SEQ ID NO: 21 - DNA, 20 bp, synthetic construct
  source 1..20: mol_type = other DNA
aagatgcacatccgaagcca

SEQ ID NO: 22 - DNA, 20 bp, synthetic construct
  source 1..20: mol_type = other DNA
tgcactgagtgtggaaaagc

SEQ ID NO: 23 - DNA, 20 bp, synthetic construct
  source 1..20: mol_type = other DNA
cgcttgacatcactgaagga

SEQ ID NO: 24 - DNA, 20 bp, synthetic construct
  source 1..20: mol_type = other DNA
catgagcgctgctcagatag

SEQ ID NO: 25 - DNA, 20 bp, synthetic construct
  source 1..20: mol_type = other DNA
cttgggatctccaacctcac

SEQ ID NO: 26 - DNA, 19 bp, synthetic construct
  source 1..19: mol_type = other DNA
ccgactctagccgcctctt

SEQ ID NO: 27 - DNA, 22 bp, synthetic construct
  source 1..22: mol_type = other DNA
cctgggcttcttctctagattg

SEQ ID NO: 28 - DNA, 23 bp, synthetic construct
  source 1..23: mol_type = other DNA
cctggtctctgtactgtccatct

SEQ ID NO: 29 - DNA, 20 bp, synthetic construct
  source 1..20: mol_type = other DNA
gcagaaggcaagcggtgaac

SEQ ID NO: 30 - DNA, 21 bp, synthetic construct
  source 1..21: mol_type = other DNA
gcgaatggagaggcagtgtac

SEQ ID NO: 31 - DNA, 20 bp, synthetic construct
  source 1..20: mol_type = other DNA
agatgtcattcctgcacacg

SEQ ID NO: 32 - DNA, 21 bp, synthetic construct
  source 1..21: mol_type = other DNA
tccctcgctgcacaaatactc

SEQ ID NO: 33 - DNA, 22 bp, synthetic construct
  source 1..22: mol_type = other DNA
cgcagacattcccgtttgtaga

SEQ ID NO: 34 - DNA, 21 bp, synthetic construct
  source 1..21: mol_type = other DNA
gcaaagggaacattccgatat

SEQ ID NO: 35 - DNA, 18 bp, synthetic construct
  source 1..18: mol_type = other DNA
tgtagccctggtgagcct

SEQ ID NO: 36 - DNA, 20 bp, synthetic construct
  source 1..20: mol_type = other DNA
ctacggcacctatcgtcacc

SEQ ID NO: 37 - DNA, 23 bp, synthetic construct
  source 1..23: mol_type = other DNA
aattgtggaattgatgcgtgatg

SEQ ID NO: 38 - DNA, 21 bp, synthetic construct
  source 1..21: mol_type = other DNA
ccaataaaggatttgacagcc

SEQ ID NO: 39 - DNA, 18 bp, synthetic construct
  source 1..18: mol_type = other DNA
atggacgaagcggatcgg

SEQ ID NO: 40 - DNA, 20 bp, synthetic construct
  source 1..20: mol_type = other DNA
caggcccagtttctgccatt

SEQ ID NO: 41 - DNA, 22 bp, synthetic construct
  source 1..22: mol_type = other DNA
ccagcaaaagcagggagtctgt

SEQ ID NO: 42 - DNA, 22 bp, synthetic construct
  source 1..22: mol_type = other DNA
gagtattactgcacagccttca

SEQ ID NO: 43 - DNA, 20 bp, synthetic construct
  source 1..20: mol_type = other DNA
gttcgccaacattgtcgtcc

SEQ ID NO: 44 - DNA, 20 bp, synthetic construct
  source 1..20: mol_type = other DNA
ccgtctcattgacttgcagc

SEQ ID NO: 45 - DNA, 21 bp, synthetic construct
  source 1..21: mol_type = other DNA
atcaaccggaggaatttccgt

SEQ ID NO: 46 - DNA, 20 bp, synthetic construct
  source 1..20: mol_type = other DNA
ggccctcaaggtttccaagg

SEQ ID NO: 47 - DNA, 23 bp, synthetic construct
  source 1..23: mol_type = other DNA
caggatcacttacggagaaacag

SEQ ID NO: 48 - DNA, 20 bp, synthetic construct
  source 1..20: mol_type = other DNA
gcaggctagagaagcagagc

SEQ ID NO: 49 - DNA, 18 bp, synthetic construct
  source 1..18: mol_type = other DNA
tgatctccaacgcctgct

SEQ ID NO: 50 - DNA, 20 bp, synthetic construct
  source 1..20: mol_type = other DNA
aaccagagccaggagacact

SEQ ID NO: 51 - DNA, 20 bp, synthetic construct
  source 1..20: mol_type = other DNA
acatcaagggcattcaggag

SEQ ID NO: 52 - DNA, 19 bp, synthetic construct
  source 1..19: mol_type = other DNA
gggaagatgctgctgttca

SEQ ID NO: 53 - DNA, 22 bp, synthetic construct
  source 1..22: mol_type = other DNA
tcaaccagaccaccttatacca

SEQ ID NO: 54 - DNA, 20 bp, synthetic construct
  source 1..20: mol_type = other DNA
ccatggtgtctgagcgatgt

SEQ ID NO: 55 - DNA, 22 bp, synthetic construct
  source 1..22: mol_type = other DNA
gtatccaagactccactccagc

SEQ ID NO: 56 - DNA, 20 bp, synthetic construct
  source 1..20: mol_type = other DNA
agctgaggttgctggttctg

SEQ ID NO: 57 - DNA, 20 bp, synthetic construct
  source 1..20: mol_type = other DNA
cgtgtggttctccactttga

SEQ ID NO: 58 - DNA, 20 bp, synthetic construct
  source 1..20: mol_type = other DNA
gcacattgacagctgagagc

SEQ ID NO: 59 - DNA, 21 bp, synthetic construct
  source 1..21: mol_type = other DNA
cttcctgaggccaataaggtg

SEQ ID NO: 60 - DNA, 21 bp, synthetic construct
  source 1..21: mol_type = other DNA
tacctggtcatgtcttcctcc

SEQ ID NO: 61 - DNA, 20 bp, synthetic construct
  source 1..20: mol_type = other DNA
cattctggatggtggacagg

SEQ ID NO: 62 - DNA, 21 bp, synthetic construct
  source 1..21: mol_type = other DNA
ttggatctcaggatggctagg

SEQ ID NO: 63 - DNA, 18 bp, synthetic construct
  source 1..18: mol_type = other DNA
ggcatccacagacatcag

SEQ ID NO: 64 - DNA, 20 bp, synthetic construct
  source 1..20: mol_type = other DNA
acagcgttgatgccagacag

SEQ ID NO: 65 - DNA, 21 bp, synthetic construct
  source 1..21: mol_type = other DNA
caaaagtcctgccacgggtct

SEQ ID NO: 66 - DNA, 21 bp, synthetic construct
  source 1..21: mol_type = other DNA
ctccaccatcagccacaggat

SEQ ID NO: 67 - DNA, 20 bp, synthetic construct
  source 1..20: mol_type = other DNA
caatgacatctaggtctccg

SEQ ID NO: 68 - DNA, 21 bp, synthetic construct
  source 1..21: mol_type = other DNA
taccacttccacctctgacga

SEQ ID NO: 69 - DNA, 19 bp, synthetic construct
  source 1..19: mol_type = other DNA
ctacagcctctccctcctt

SEQ ID NO: 70 - DNA, 19 bp, synthetic construct
  source 1..19: mol_type = other DNA
cagtttgggccccttcaaa

SEQ ID NO: 71 - DNA, 22 bp, synthetic construct
  source 1..22: mol_type = other DNA
gtgtccatgatgatgctcattt

SEQ ID NO: 72 - DNA, 20 bp, synthetic construct
  source 1..20: mol_type = other DNA
gtcgagggaaaaataggctg

SEQ ID NO: 73 - DNA, 22 bp, synthetic construct
  source 1..22: mol_type = other DNA
cactattgacgtttccccactc

SEQ ID NO: 74 - DNA, 20 bp, synthetic construct
  source 1..20: mol_type = other DNA
cattcgggagaaggtccgag

SEQ ID NO: 75 - DNA, 20 bp, synthetic construct
  source 1..20: mol_type = other DNA
tggtgatgctgaaagagacg

SEQ ID NO: 76 - DNA, 20 bp, synthetic construct
  source 1..20: mol_type = other DNA
cttgccacactctgtgcatt

SEQ ID NO: 77 - DNA, 20 bp, synthetic construct
  source 1..20: mol_type = other DNA
acacgcaaatttccttccac

SEQ ID NO: 78 - DNA, 20 bp, synthetic construct
  source 1..20: mol_type = other DNA
aggagcagcagctcttcttg

SEQ ID NO: 79 - DNA, 21 bp, synthetic construct
  source 1..21: mol_type = other DNA
gtggaagtcgcgttctttaca

SEQ ID NO: 80 - DNA, 22 bp, synthetic construct
  source 1..22: mol_type = other DNA
ttgtcttggaaaggttgttctg

SEQ ID NO: 81 - DNA, 22 bp, synthetic construct
  source 1..22: mol_type = other DNA
gatcacgaacagctgtgaagtc

SEQ ID NO: 82 - DNA, 20 bp, synthetic construct
  source 1..20: mol_type = other DNA
tagcaggacagcagatgagt

SEQ ID NO: 83 - DNA, 26 bp, synthetic construct
  source 1..26: mol_type = other DNA
gagtgacgttgtcttcattagcactg

SEQ ID NO: 84 - DNA, 20 bp, synthetic construct
  source 1..20: mol_type = other DNA
aaggagaagcttcctcagcc

SEQ ID NO: 85 - DNA, 19 bp, synthetic construct
  source 1..19: mol_type = other DNA
ttctgcccctgccaaatct

SEQ ID NO: 86 - DNA, 24 bp, synthetic construct
  source 1..24: mol_type = other DNA
gtgaccgggaatatcaggaacaag

SEQ ID NO: 87 - DNA, 21 bp, synthetic construct
  source 1..21: mol_type = other DNA
gcgacatcacatggaaatcta

SEQ ID NO: 88 - DNA, 18 bp, synthetic construct
  source 1..18: mol_type = other DNA
ccaacggtcagcatcaca

SEQ ID NO: 89 - DNA, 20 bp, synthetic construct
  source 1..20: mol_type = other DNA
ctgcggctgcttctctatga

SEQ ID NO: 90 - DNA, 23 bp, synthetic construct
  source 1..23: mol_type = other DNA
ctacaacgatcccctctgaaaaa

SEQ ID NO: 91 - DNA, 20 bp, synthetic construct
  source 1..20: mol_type = other DNA
gcatctgtgtcattgatggg

SEQ ID NO: 92 - DNA, 18 bp, synthetic construct
  source 1..18: mol_type = other DNA
ccctggccttatgatgtt

SEQ ID NO: 93 - DNA, 21 bp, synthetic construct
  source 1..21: mol_type = other DNA
ttccagctcagcgtggtcgta

SEQ ID NO: 94 - DNA, 25 bp, synthetic construct
  source 1..25: mol_type = other DNA
tgtctgtgtcatcggagtgatatcc

SEQ ID NO: 95 - DNA, 22 bp, synthetic construct
  source 1..22: mol_type = other DNA
aaccactgcaataagtcctttc

SEQ ID NO: 96 - DNA, 23 bp, synthetic construct
  source 1..23: mol_type = other DNA
gtagttcttcttgtcgtagtcgc

SEQ ID NO: 97 - DNA, 20 bp, synthetic construct
  source 1..20: mol_type = other DNA
gatgtggtcccctcattcgt

SEQ ID NO: 98 - DNA, 21 bp, synthetic construct
  source 1..21: mol_type = other DNA
caccaggacgaccaggttttc

SEQ ID NO: 99 - DNA, 21 bp, synthetic construct
  source 1..21: mol_type = other DNA
caccctgtggtccaacaactc

SEQ ID NO: 100 - DNA, 22 bp, synthetic construct
  source 1..22: mol_type = other DNA
gccagtgacagcatacacagtg

SEQ ID NO: 101 - DNA, 20 bp, synthetic construct
  source 1..20: mol_type = other DNA
atgtcttcatgctggtgcag

SEQ ID NO: 102 - DNA, 20 bp, synthetic construct
  source 1..20: mol_type = other DNA
tcatgttcaggtccaactcg

SEQ ID NO: 103 - DNA, 20 bp, synthetic construct
  source 1..20: mol_type = other DNA
gagacctctggcttcgtcag

SEQ ID NO: 104 - DNA, 21 bp, synthetic construct
  source 1..21: mol_type = other DNA
gcctccgtataccgcatcaat

SEQ ID NO: 105 - DNA, 20 bp, synthetic construct
  source 1..20: mol_type = other DNA
tcaactcactccgggaactc

SEQ ID NO: 106 - DNA, 17 bp, synthetic construct
  source 1..17: mol_type = other DNA
atccgcagacactccat

(19) United States
(12) Patent Application Publication
(10) Pub. No.: US 2026/0159794 A1
(43) Pub. Date: Jun. 11, 2026

(54) Advanced Placenta On A Chip

(71) Applicant: The Trustees of the University of Pennsylvania, Philadelphia, PA (US)

(72) Inventors: Dongeun Huh, Villanova, PA (US); Mousa Younesi, Philadelphia, PA (US); Pouria Fattahi, Philadelphia, PA (US)

(21) Appl. No.: 19/347,379

(22) Filed: Oct. 1, 2025

Related U.S. Application Data
(60) Provisional application No. 63/702,039, filed on Oct. 1, 2024.

(51) Int. Cl. C12M 3/00; C12M 1/00; C12M 1/12; C12N 5/073

(52) U.S. Cl. CPC C12M 21/08; C12M 23/40; C12M 25/04; C12N 5/0605; C12N 2502/28; C12N 2513/00

(57) ABSTRACT

A placenta-on-a-chip fluidic chip, comprising: a bottom region, the bottom region comprising a central channel and at least one side channel adjacent thereto, the at least one side channel having therein a plurality of placental or placenta-derived cells, the central channel having therein a plurality of cells disposed in a matrix; and a upper region, the upper region having an upper channel defined therein, the upper channel being in register with at least one of the central channel and the at least one side channel of the bottom region. A method, comprising communicating an agent within the upper channel of a fluidic chip according to the present disclosure and monitoring a response of at least one of a tissue in the upper channel and a tissue in the central channel.

### RELATED APPLICATIONS

The present application claims priority to and the benefit of U.S. patent application No. 63/702,039, “Advanced Placenta On A Chip” (filed Oct. 1, 2024). All foregoing applications are incorporated herein by reference in their entireties for any and all purposes.

### GOVERNMENT RIGHTS

This invention was made with government support under ES029275 awarded by the National Institutes of Health. The government has certain rights in the invention.

### SEQUENCE LISTING

The instant application contains a Sequence Listing which is being submitted herewith electronically in XML format and is hereby incorporated by reference in its entirety. Said XML copy, created on Jan. 14, 2026, is named 103241024776_22-10105_SequenceListing_ST26.xml and is 94,374 bytes in size.

### TECHNICAL FIELD

The present disclosure relates to the field of organ-on-a-chip devices.

### BACKGROUND

Organs-on-a-chip have emerged as an alternative to animal testing in the biotechnology and pharmaceutical companies. A placenta-on-a-chip has particular utility, but because existing placenta-on-a-chip designs can have suboptimal characteristics in some cases, there is a long-felt need in the art for improved placenta-on-a-chip designs.

### SUMMARY

This advanced placenta-on-a-chip system incorporates a vascularized interface at the maternal channel which allows for more realistic evaluation of the actual interface between the mother and placenta.

In meeting the described long-felt needs, the present disclosure provides a placenta-on-a-chip fluidic chip, comprising: a bottom region, the bottom region comprising a central channel and at least one side channel adjacent thereto, the at least one side channel having therein a plurality of placental or placenta-derived cells, the central channel having therein a plurality of cells disposed in a matrix; and a upper region, the upper region having an upper channel defined therein, the upper channel being in register with at least one of the central channel and the at least one side channel of the bottom region.

Also provided is a method, comprising communicating an agent within the upper channel of a fluidic chip according to the present disclosure and monitoring a response of at least one of a tissue in the upper channel and a tissue in the central channel.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 depicts a chip according to the present disclosure. As shown, a chip according to the present disclosure can comprise a fluidic path that corresponds to material circulation and a fluidic path that corresponds to fetal circulation. As shown in the bottom-right image of FIG. 1, a chip can include a main media channel or side channel, which main media channel can be lined with endothelial from the placenta. A chip can also include a central channel, which can include therein perfusable vessels formed with endothelial and fibroblast in ECM. As shown, a rail or ridge can be present between the side channel and the central channel. A material channel can be present and be in fluid communication—for example, across a membrane—from the central channel. The material channel can be lined with trophoblast from the placenta.

FIG. 2 provides exemplary images of cellular constructs formed using the disclosed technology.

FIGS. 3A-3R. A bioengineered microphysiological model of the maternal-fetal interface in the human placenta. FIG. 3A, 3B. Schematics of the cotyledon in the human placenta (FIG. 3A) and the placental barrier (FIG. 3B). FIG. 3C. Illustration of a microengineered 3D model of the human placental barrier consisting of i) the maternal compartment designed to mimic the maternal blood-containing intervillous space and the trophoblast layer of the placental barrier and ii) the fetal compartment that represents a vascularized connective tissue of the placental barrier with fetal circulation. Please note that trophoblasts and the other cell types in the connective tissue are of fetal origin. FIG. 3D. Layer-by-layer view of the microdevice used to culture placental cells. FIG. 3E. A sequential process of human placental tissue production in the device. FIG. 3F. Illustration of primary human placental cells sourced from placenta explants for use in our model. FIG. 3G. Representative confocal images capturing the progression of vascular formation over a 7-day period. Scale bar, 100 μm. FIG. 3H, FIG. 3I. Quantification of select vascular features during vasculogenesis. FIG. 3J, FIG. 3K. Demonstration of vascular perfusion using red blood cells (RBCs) (FIG. 3J) and 70 kDa FITC-dextran (pseudo-colored black in the timelapse images) (FIG. 3K). Scale bar, 20 μm. FIG. 3L. A confocal micrograph of microvilli shown with actin immunostaining (green). Scale bar, 25 μm. FIG. 3M. E-cadherin (red) and nuclear (blue) staining before (Day 7) and after (Day 10) spontaneous syncytialization. Day 7 represents the day of trophoblast seeding and monolayer formation. E-cadherin staining decreased between Day 7 and Day 10 due to cell fusion and the resultant loss of intercellular junctions. During syncytialization, the trophoblast layer became slightly thinner and as a result, the apical surface of the cell layer at Day 10 in this image was captured roughly 6 μm above the membrane surface. Scale bar, 50 μm. FIG. 3N. Quantification of nuclear area in the trophoblast layer. FIG. 3O. Quantification of the level of β-human chorionic gonadotropin (β-hCG) during syncytialization over 72 hours from Day 7 through Day 10 (left), at different oxygen conditions (middle), and under static vs. dynamic culture conditions (right). FIG. 3P. A heatmap of gene expression analyzed by RT-PCR. FIG. 3Q. Comparison of barrier integrity using transepithelial electrical resistance (TEER). FIG. 3R. Immunofluorescence of GLUT-1 (left) and quantification of the rate of glucose transfer from the maternal to fetal chambers (right). Scale bar, 25 μm. Data are presented as mean±SD with n=3, 4. ns: not significant, *P<0.05, **P<0.01, ***P<0.001, and ****P<0.0001.

FIGS. 4A-4X. Simulation of cadmium exposure in the engineered placental model. FIG. 4A. Schematic illustrations of in vitro techniques to model exposure of the in vivo human placental barrier to cadmium using our platform. FIG. 4B, 4C. Fluorescence micrographs of trophoblasts exposed to varying concentrations of cadmium chloride in the engineered model (FIG. 4B) and Transwell (FIG. 4C). Green and red fluorescence show live and membrane-compromised cells, respectively. Scale bar, 20 μm. FIG. 4D. Quantification of LDH release from the maternal compartment after 24 hours of cadmium exposure. ‘Device-static’ and ‘Device-dynamic’ represent cadmium exposure in the engineered model in the presence and absence of flow, respectively. Data are normalized to the untreated control group (0 μM). FIG. 4E. Plots of caspase-3/7 activities after 24 hours of exposure to varying cadmium concentrations (left) and at 10 μM (right). FIG. 4F. Quantification of maternal-to-fetal cadmium transfer. FIG. 4G. Quantification of LDH release from the fetal compartment after 24 hours of cadmium exposure. ‘Device-static’ and ‘Device-dynamic’ represent cadmium exposure in the engineered model in the presence and absence of flow, respectively. Data are normalized to the untreated control group (0 μM). FIG. 4H. Effects of cadmium on barrier function as assessed by TEER (left), permeability to fluorescently labeled bovine serum albumin (BSA) (middle), and permeability to lucifer yellow (LY) dye (right). LY data were normalized to the untreated control group (0 μM). FIG. 1. Quantification of β-hCG secretion by trophoblasts in the maternal chamber. FIG. 4J. Immunofluorescence imaging of ZO-1 (left column) and GLUT1 (right column) transporters expressed by trophoblasts. Scale bar, 20 μm. FIG. 4K. Evaluation of the rate of maternal-to-fetal glucose transfer. Data are presented as mean±SD with n=3, 4 and 6. ns=not significant, *P<0.05, **P<0.01, ***P <0.001, and ****P<0.0001. FIG. 4L, 4M. Responses of engineered placental barrier to cadmium. FIG. 4A, 4B. Analysis of select pro-inflammatory cyto-kines in media effluent collected from the maternal (FIG. 4L) and fetal (FIG. 4M) compartments of our model (labeled “Device”) and Transwell. n,o. Comparison of ICAM-1 expression in the vasculature (FIG. 4N) and vascular permeability (FIG. 4O). Scale bars, 100 μm. FIG. 4P. Schematic illustration of Hofbauer cells present in the maternal-fetal interface in vivo. FIG. 4Q. Immuno-fluorescence imaging of CD163+ primary human Hofbauer cells incorporated into the engineered placental barrier model and grown for seven days. Scale bar, 20 μm. FIG. 4R-4T, Evaluation and comparison of cytokine production in the presence or absence of Hofbauer cells (HBC). FIG. 4U, 4V. Elevated expression of α-SMA (u) and increased production of fibronectin and collagen (FIG. 4V) due to cadmium exposure. The punctate pattern of α-SMA staining in the micrographs in j represents non-specific background signal, rather than actual protein expression. Scale bars, 100 μm. 1,m. Heatmaps illustrating altered gene expression in the maternal (FIG. 4W) and fetal (FIG. 4X) compartments due to cadmium analyzed by RT-PCR. Data are presented as mean±SD with n=3, 4. ns=not significant, *P<0.05, **P<0.01, ***P<0.001, and ****P<0.0001.

FIGS. 5A-5N. Investigation of BCRP in the cadmium exposure model. FIG. 5A. Schematics of efflux transporters ex-pressed on the maternal blood-facing apical surface of trophoblasts in the human placental barrier. FIG. 5B, 5C. Quantification of cadmium-induced LDH release (FIG. 5B) and pro-inflammatory cytokine production (FIG. 5C) when efflux transporters are biochemically inhibited. Data in (FIG. 5B) were normalized to the untreated control group (not shown in the plot). FIG. 5D. Illustration of incorporating BeWo cells with BCRP knockdown into the engineered placental barrier model. FIG. 5E. Comparison of BCRP expression in the wild-type (WT) and BCRP knockdown (KD) models. Scale bar, 50 μm. The plot shows relative expression of BCRP measured by RT-PCR. f,g. Evaluation of β-hCG secretion in the maternal compartment (FIG. 5F) and barrier permeability to lucifer yellow (LY) (FIG. 5G) after 24 hours of exposure to cadmium. Data were normalized to the untreated control group (0 μM). FIG. 5H, FIG. 5I. Comparison of LDH release (FIG. 5H) and pro-inflammatory cytokine production (FIG. 5I) in the WT and KD models after 24-hour cadmium exposure. Data in (FIG. 5H) were normalized to the untreated control group (0 μM). FIG. 5J-FIG. 5L. Comparison of endothelial expression of ICAM-1 (FIG. 5J), fibroblast expression of α-SMA (FIG. 5K), and fibronectin deposition in the stroma (FIG. 5L) after 24-hour exposure to 5 μM cadmium. Scale bar, 100 μm. FIG. 5M. Demonstration of neutrophil adhesion to the vasculature due to cadmium exposure at 5 μM for 24 hours. Scale bar, 100 μm. FIG. 5N. Comparison of cadmium-induced pro-inflammatory cytokine production in the maternal (top row) and fetal (bottom row) compartments in the presence or absence of neutrophils. Data are presented as mean±SD with n=4. ns=not significant, *P<0.05, **P<0.01, ***P<0.001, and ****P<0.0001.

FIGS. 6A-6K. Metabolomic profiling of cadmium-exposed maternal compartment. FIG. 6A. Schematic illustration of metabolomic analysis of device effluent collected from the maternal compartment of the engineered placental barrier model. FIG. 6B, FIG. 6C. PCA plot (FIG. 6B) and heatmap showing relative abundance of top 183 metabolites (FIG. 6C) in all conditions tested. FIG. 6D, FIG. 6E. Quantification of select metabolite upregulated (FIG. 6D) or downregulated (FIG. 6E) by cadmium exposure. The color of the box label represents the type of metabolites (blue: involved in peptide metabolism; red: carbohydrate metabolism; grey: lipid metabolism; green: nucleic acid metabolism). Data were normalized to the median. FIG. 6F. Plot of significantly changed metabolic pathways identified by pathway impact analysis of our model exposed to 10 μM cadmium for 24 hours FIG. 6G. ROC curves for biomarker prediction models. Different colors represent models with different numbers of constituent metabolite features. FIG. 6H. The list of top metabolites generated by the 25-feature prediction model and ranked based on their predictive accuracy. FIGS. 6I-6k. Pathway impact plot (FIG. 6I), ROC curves (FIG. 6J), and the list of top metabolite biomarker candidates (FIG. 6K) generated by metabolomic analysis of our model exposed to 1 μM cadmium for 24 hours. Box plots show minimum, 25^{th} percentile, mean, 75^{th} percentile, and maximum values. Data are presented as mean±SD with n=5. ns=not significant, *P<0.05, **P<0.01, ***P<0.001, and ****P<0.0001.

FIGS. 7A-7K. Metabolomic profiling of cadmium-exposed fetal compartment. FIG. 7A. Schematic illustration of metabolomic analysis of device effluent collected from the fetal compartment of the engineered placental barrier model. FIGS. 7B, 7C. PCA plot (FIG. 7B) and heatmap showing relative abundance of top 255 metabolites (FIG. 7C) in all conditions tested. FIGS. 7D, 7E. Quantification of select metabolite upregulated (FIG. 7D) or downregulated (FIG. 7E) by cadmium exposure. The color of the box label represents the type of metabolites (blue: involved in peptide metabolism; red: carbohydrate metabolism; grey: lipid metabolism; green: nucleic acid metabolism). Data are normalized to the sample median. FIG. 7F. Plot of significantly changed metabolic pathways identified by pathway impact analysis of our model exposed to 10 μM cadmium for 24 hours. FIG. 7G. ROC curves for biomarker prediction models. Different colors represent models with different numbers of constituent metabolite features. FIG. 7H. The list of top metabolites generated by the 25-feature prediction model and ranked based on their predictive accuracy. FIGS. 7I-7K. Pathway impact plot (FIG. 7I), ROC curves (FIG. 7J), and the list of top metabolite biomarker candidates (FIG. 7K) generated by metabolomic analysis of our model exposed to 1 μM cadmium for 24 hours. Box plots show minimum, 25^{th} percentile, mean, 75^{th} percentile, and maximum values. Data are presented as mean±SD with n=5. ns=not significant, *P<0.05, **P<0.01, ***P<0.001, and ****P<0.0001.

FIGS. 8A-8M. Ex vivo model of human placental exposure to cadmium. FIG. 8A. Schematics and photo of villous explants from whole human placentas grown in a perfusion chamber to create an ex vivo model. FIG. 8B. Micrograph of placental explants cultured in our device for 48 hours (left) and quantification of tissue viability and LDH release (right). Green and red fluorescence in the micrograph show live and dead cells, respectively. Tissue viability was quantified as the percentage of live cells in the total population. Scale bar, 200 μm. FIG. 8C. Hematoxilyn and Eosin (H&E) and Masson's Trichrome (MT) staining of histological sections of villous explants. Scale bar, 100 μm. FIG. 8D. Immunofluorescence im-age showing the key structural components of the placental barrier. Scale bars, 50 μm. FIG. 8E. Timeline for cadmium treatment in the explant model. FIG. 8F. Live/dead staining of villous explants in our device after 24 hours of cadmium exposure. Ctrl represents a control group without cadmium treatment. Scale bars, 200 μm. FIG. 8G, FIG. 8H. Quantification of tissue viability and LDH release (FIG. 8G) and cytokine production (FIG. 8H) following 24-hour cadmium treatment of the ex vivo model. FIG. 8I. Illustration of LC-MS-based untargeted metabolomic analysis of device effluent from the ex vivo model. FIG. 8J, FIG. 8K. PCA (FIG. 8J) and heatmap (FIG. 8K) representation of the metabolomics data. FIG. 8K. Heatmap showing relative abundance of detected metabolites across cadmium doses. FIG. 8L. Comparison of significantly altered metabolites commonly found in the ex vivo and in vitro models treated with 10 μM cadmium (The graphs for the in vitro model were generated using the same data presented in FIGS. 6 and 7 and FIGS. 21 and 24.). FIG. 8M. Plot of significantly altered metabolic pathways identified by pathway impact analysis following 24-hour exposure of the explant model to 10 μM cadmium. Data are presented as mean±SD with n=3, 4. ns=not significant, *P<0.05, **P<0.01, ***P<0.001, and ****P<0.0001.

FIG. 9. Comparison of cadmium-induced production of pro-inflammatory cytokines between the ex-plant and microengineered in vitro exposure models. The graphs for the in vitro model were generated using the same data presented in FIGS. 4a and 4b. Data are presented as mean±SD with n=4. ns=not significant, *P<0.05, ***P<0.001, and ****P<0.0001.

FIG. 10. Metabolic pathways significantly altered by cadmium exposure at 10 μM that are commonly found in all three groups. For visualization purposes, all the other pathways have been shaded. The plots for the maternal and fetal compartments of the in vitro model are the same as those shown in FIGS. 6f and 7f, respectively.

FIG. 11. Injection and spatial confinement of cell-ECM mixture. Time-series images demonstrating the injection of cell-ECM mixture solution into the fetal chamber of the device. Injection only occurs in the bottom layer separated from the top compartment by a permeable membrane—the top and membrane layers are not clearly visible in these images. Scale bars, 1 mm.

FIGS. 12A-12C. Analysis of fetal vasculature in human placental villi explants. FIG. 12A. Immunofluorescence micrographs of chorionic villi isolated from human placental explants. The white arrows in the magnified view indicate the fetal blood vessels. Scale bar, 50 μm. FIG. 12B. Histogram showing the frequency distribution of the diameter of blood vessels detected in immunofluorescence images. The mean diameter is 20.3+11.7 μm. FIG. 12C. Comparison of the vessel diameter. HPVEC and HUVEC represent human placental villous endothelial cell and human umbilical vein endothelial cell, respectively. The “Placental explant vessels” group shows the diameter of blood vessels detected in human placental explants. Data are presented as mean±SD with n=4.

FIGS. 13A-13B. Effect of flow culture conditions on placental vasculature. FIG. 13A. Representative confocal images comparing vascular formation under static and dynamic conditions on day 7 (left) and quantification of select vascular features (right). Green fluorescence in the confocal images shows immunostaining of CD31 expressed by endothelial cells (scale bar=100 μm). FIG. 13B. Comparison of microvilli formation on the apical surface of trophoblasts under static and flow culture conditions. Microvilli in the confocal images are shown by immunofluorescence staining of actin. Blue shows nuclear staining. The micrograph showing a top view of microvilli in the dynamic culture condition was repeated from FIG. 3I. Data are presented as mean±SD with n=3, 4. **P<0.01, and ****P<0.0001.

FIGS. 14A-14C. Plasmonic detection and quantification of cadmium. FIG. 14A. Gold nanoparticles (AuNPs) functionalized with carboxylic acid-terminated poly (ethylene glycol) (PEG) surface groups were generated using a sodium citrate reduction method. In the presence of cadmium, the distance between these particles decreases due to the chelation of cadmium ions by the carboxylate terminals of PEG, providing a basis for plasmonic detection of cadmium in our effluent samples. FIG. 14B. Absorbance spectra measured after mixing AuNP-containing culture media with different concentrations of cadmium ions. As the concentration of cadmium increases, the intensity of the surface plasmon resonance (SPR) band at 535 nm (Aₘₐₓ) decreases, whereas SPR intensity at 800 nm (A800) increases. FIG. 14C. During 24-hour cadmium exposure in our model, device effluent was collected from the maternal and fetal chambers of our device and mixed with AuNP solution. Subsequently, A800 and Aₘₐₓ were measured for both compartments, and their ratios (shown as M and F in the plot) were used in conjunction with the known concentration of cadmium in the maternal chamber (Cdₘₐₜₑᵣₙₐₗ) to calculate the cadmium concentration in the fetal chamber (Cd_{fetal}), which also al-lowed us to estimate the rate of maternal-to-fetal cadmium transfer shown in FIG. 4f.

FIG. 15. Effect of cadmium exposure on E-cadherin expression. Representative immunofluorescence images of E-cadherin expression (red) by trophoblasts in our model after 24-hour exposure to cadmium at 0 (control), 5, and 10 μM. Quantification shows cadmium-induced significant reduction in E-cadherin expression compared to the negative control. Blue shows nuclear staining. Data are presented as mean±SD with n=5. ns=not significant, **P<0.01, and ****P<0.0001. Scale bar, 50 μm.

FIG. 16. Positive control for analysis of cadmium-induced inflammation. Quantification of IL-1B, IL-6, and IL-8 levels in the maternal and fetal compartments of the placental model after 24-hour treatment with TNF-α at a physiological concentration of 10 ng/ml as a positive control for the analysis of cadmium-induced inflammatory responses. In each plot, cytokine levels following TNF-α treatment are shown alongside those induced by cadmium treatment at 10 μM to provide a reference point for comparison. Data for the control and 10 μM group were repeated from FIGS. 4a and 4b. Data shown as mean±SD, n=4. ns=not significant, *P<0.05, **P<0.01, ***P<0.001, and ****P<0.0001.

FIG. 17. Comparison of vascular permeability. Representative confocal images showing perivascular diffusion of fluorescently labeled 70 kDa dextran injected into microengineered blood vessels derived from HUVECs (left) or human placental villous endothelial cells (HPVECs) (right) in the stromal compartment of the placental model. The grey horizontal labels indicate the concentration of cadmium chloride introduced into the maternal compartment. Green fluorescence shows blood vessels in both image sets. Data are presented as mean±SD with n=5. ns=not significant, and ****P<0.0001. Scale bars, 50 μm.

FIG. 18. Cadmium-induced gene expression changes. Quantification of relative expression of top-ranked genes in the maternal (top) and fetal (bottom) compartments via RT-PCR, following 24-hour exposure of our placental model to cadmium at 0, 1 μM, and 10 μM. The bar graphs were generated using the same data presented in FIGS. 4l and 4m. Data are presented as mean±SD, n=3. ns=not significant, *P<0.05, **P<0.01, ***P<0.001, and ****P<0.0001.

FIG. 19. Heatmap of metabolites in the maternal compartment. A heatmap showing the relative abundance of the top 183 metabolites detected in all groups tested. Each column within a given group represents an independent replicate.

FIG. 20. Metabolomic profiles of cell-free model-maternal compartment. Plot of principal component analysis (top) and hierarchical clustering heatmap (bottom) comparing metabolomic profiles of the maternal compartment in our microengineered placental model with-out cadmium treatment—labelled “Untreated control”—and a cell-free model created using the same device design that contained the same hydrogel scaffold and culture media without any cells-labelled “Cell-free control”. n=5 replicates per condition.

FIGS. 21A-21F. Comparison of select metabolites in the maternal compartment. FIGS. 21A-21B, Volcano plots showing the top differentially regulated metabolites due to cadmium exposure at 10 μM (FIG. 21A) or 1 μM (FIG. 21B). FIG. 21C-FIG. 21D, Boxplots showing the normalized concentrations of select carbohydrate (FIG. 21C), nucleotide (FIG. 21D), peptide (FIG. 21E), and lipid (FIG. 21F) metabolites detected in the maternal compartment. Each box shows minimum, 25^{th} percentile, mean, and 75^{th} percentile. Ctrl represents an untreated negative control group. Data were normalized to the median. Data show mean±SD with n=5 for each group. ns=not significant, *P<0.05, **P<0.01, ***P<0.001, and ****P<0.0001.

FIG. 22. Heatmap of metabolites in the fetal compartment. A heatmap showing the relative abundance of top 255 metabolites detected in all groups tested. Each column within a given group represents an independent replicate.

FIG. 23. Metabolomic profiles of cell-free model—fetal compartment. Plot of principal component analysis (top) and hierarchical clustering heatmap (bottom) comparing metabolomic profiles of the fetal compartment in our microengineered placental model without cadmium treatment—labelled “Untreated control”—and a cell-free model created using the same device design that contained the same hydrogel scaffold and culture media without any cells—la-belled “Cell-free control”. n=5 replicates per condition.

FIGS. 24A-24F. Comparison of select metabolites in the fetal compartment. FIG. 24A-24B, Volcano plots showing top differentially regulated metabolites due to cadmium exposure at 10 μM (FIG. 24A) or 1 μM (FIG. 24B). FIG. 24C-24D, Boxplots showing the normalized concentrations of select carbohydrate (c), nucleotide (FIG. 24D), peptide (FIG. 24E), and lipid (FIG. 24F) metabolites detected in the fetal compartment. Each box shows minimum, 25^{th} percentile, mean, and 75^{th} percentile. Ctrl represents an untreated negative control group. Data are normalized to the median. Data show mean±SD with n=5 for each group. ns=not significant, *P<0.05, **P<0.01, ***P<0.001, and ****P<0.0001.

FIG. 25 provides a depiction of the disclosed technology at a first point (e.g., Day 0) in time, showing a device having a polydopamine (PDA) coating and various features of the device. As shown, a device can include a maternal chamber that is defined by a pervious membrane—such as a porous membrane having a pore size of about 1 micrometer. The device can also include a vascular chamber, which vascular chamber can be separated from the material chamber by the pervious membrane. One or more media channels can be adjacent to the vascular chamber; a rail can define the border between a media channel and the vascular chamber. A cell-laden extracellular matrix (ECM) material can be disposed within the vascular chamber.

FIG. 26 provides a depiction of the disclosed technology at a second point in time (e.g., Day 1). As shown, the central channel can have disposed therein an ECM hydrogel that includes endothelial cells and fibroblasts.

FIG. 26 provides a depiction of the disclosed technology at a third point in time (e.g., Day 2). As shown, the media channel can include endothelial material.

FIG. 27 provides a depiction of the disclosed technology at a third point in time (e.g., Day 2). As shown, the media channel can include endothelial material.

FIG. 28 provides a depiction of the disclosed technology at a fourth point in time (e.g., Day 7). As shown, the central channel can have disposed therein a vascular network, which vascular network can place media channels into fluid communication with one another.

FIG. 29 provides a depiction of the disclosed technology at a fifth point in time (e.g., Day 7-10). As shown, the maternal compartment can be lined with synctiotrophoblast, and the central channel (fetal compartment) can be lined with endothelial material from term-placenta.

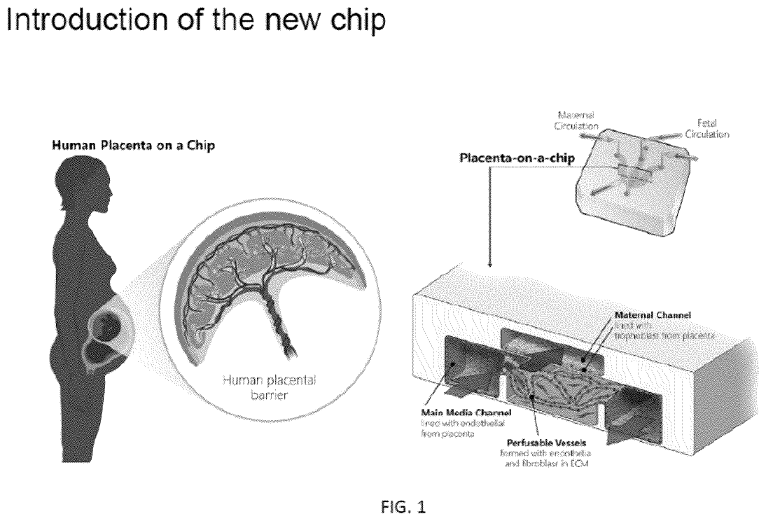

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

### Background

Exposure to toxic heavy metals contributes to the development of pregnancy complications that can adversely affect fetal development. Despite increasing evidence suggesting the negative health impact of heavy metals, investigating their adverse effects in the context of human pregnancy remains a major challenge due to the difficulty of human subject research and the limited capacity of animal models to properly represent the anatomy and function of the human reproductive system.

Motivated by this problem, here we describe two microengineered in vitro models designed to reverse engineer the maternal-fetal interface in the human placenta. In our first model, a biomimetic system enables advanced capabilities to grow human trophoblast cells and fetal vascular endothelial cells in a physiological spatial arrangement and dynamic culture conditions to engineer the placental barrier in vitro that can emulate regulated material transport between maternal and fetal circulations.

In the second model, a more complex microengineered model is developed capable of biological resemblance to human placental. We use this chip to model the interface of human placental barrier, which includes fully syncytialized trophoblast and 3D vascular endothelial network.

Using cadmium (CdCl2) as a representative example of heavy metals, we investigate whether and how this environmental chemical affects the human placental barrier and its physiological function as a gatekeeper. Our study also examines the role of membrane transporters including the breast cancer resistance protein (BCRP) in the maternal-to-fetal transfer of cadmium to provide insights into efflux transporter-medicated protection against environmental exposures during pregnancy.

### 1. Vascularized Placenta-On-a-Chip Device Design and Construction

Vascularized placenta-on-a-chip (VPOC) device composed of two compartmentalized fluidic devices separate by a semipermeable membrane. The bottom compartment consists of two parallel microchannels defined by one microguide rail protruding from the bottom of the lower microdevice. Both compartmentalized fluidic devices are fabricated with polydimethylsiloxane (PDMS) using standard soft lithography protocols. An ECM hydrogel scaffold capable of encapsulating multiple fetal cell types injected into the middle channel and later form a scaffold for microvascular network.

After device is assembled and sterilized, a fibrin solution mixed with villus endothelial cells (VEC) and fibroblast is injected into the middle microchannel (dimeter: 2000 μm and height: 400 μm) next to microguide rail. Cell-laden mixture is confined in the central channel due to the pinning effect of PDMS microguide rail and preventing outflow to the side channel. Side channels are seeded with endothelial cells the next day and after one week of vascular network formation the top compartment is seeded with trophoblast cells.

### 2. Cellular Components in VPOC

To replicate a physiologically relevant system that emulates the cellular microenvironment of placenta barrier, primary trophoblasts are obtained from human tissue (Amnion Foundation) to form maternal side and villus endothelial cells and fibroblast isolated from term placenta are used to form fetal microvascular.
Step 1: Primary human fibroblasts and villus microvascular endothelial cells are suspended in an ECM hydrogel solution at 5×10⁶ cells/ml and 2.5×10⁶ cells/ml, respectively. The cell and gel mixture are injected into the central lane of the bottom chamber. Side channels are seeded with endothelial cells next day and cells are grown for 7 days to form a perfusable microvascular network reminiscent of sub-epithelial stroma.
Step 2: For maternal side, we use primary trophoblasts isolated from term-placenta. Primary trophoblasts are be seeded into the top compartment and grow to form confluent monolayers over 3 days. The bi-layer tissue was then cultured under perfusion conditions by using syringe pumps connected to each chamber. To mimic the physiological process of placental development, we treated the trophoblast cell layer in the maternal chamber with forskolin (50 μM) to induce syncytialization.

### 3. Evaluation of Heavy Metal Toxicity in Placenta

The syncytialized maternal tissue was then exposed to the flow of medium containing cadmium (0.5-50 μM), and trophoblast responses were analyzed using various techniques including microfluorimetry, ELISA, and PCR.

### Preliminary Findings

### 1. Increased Invasiveness of Primary EVTs in the Presence of Endothelial Cells

Our microengineered model of the human placental barrier formed a tight barrier (evaluated by expression of E-cadherin and VE-cadherin), secreted the pregnancy hormone human chorionic gonadotropin beta (hCGB), and supported physiological maternal-to-fetal transport of glucose. Following CdCl2 treatment, we observed concentration-dependent deleterious responses of the maternal and fetal tissues as demonstrated by reduced cell viability and placental hormone secretion, compromised barrier function, and increased secretion of proinflammatory cytokines. The results also indicated upregulated expression of metallothioneins IA and IIA (MT-IA/IIA) metal binding proteins.

Progress in our understanding of how placenta barrier protect fetus from potentially noxious substances has been hindered by the absence of appropriate models that accurately replicate human placenta barrier. Animal model, ex-vivo tissue and simple in vitro models have been the main modalities to study protection mechanism of the placenta barrier. However, each of these methods has inherent limitations, and there is a substantial need for an alternative yet novel in vitro model capable of mimicking the placenta barrier function to study the adverse effect of noxious substances on fetus.

VPOC device emerge as a powerful research tool to emulate the placenta barrier not only to study the function of this complex tissue but also investigate the role of environmental toxic materials on this tissue interface.

Using VPOC model we can precisely model tissue heterogeneity and 3D architecture of the maternal-fetal interface, such complex yet novel engineered construct is enabled by multilayer compartmentalized design of the microchip and our unique techniques for forming 3D vascular hydrogel scaffolds. The trophoblasts in our chip are syncytialized just like maternal cells in human placenta.

Our chip is made from transparent, and easily fabricated elastomer which is ideal for advanced microscopy and analysis.

This technology thus provides the ability to (1) study and understand the placenta functional, (2) explore the mechanism by which the placenta protects the fetus, and (3) can be used as a screening tool to test therapeutic effect of drugs.

Finally, we used 4 different patient-derived cells in our VPOC which makes this chip as a candidate for personalized tool to study human-specific diseases.

### Additional Disclosure

Exposure of pregnant women to toxic metals is an environmental health issue associated with various pregnancy complications. Efforts to advance our biological understanding of this problem and mitigate its adverse effects, however, have been challenged by ethical concerns of human subject research during pregnancy. Here, we present an alternative approach that leverages the design flexibility, controllability, and scalability of bioengineered human reproductive tissues to enable experimental simulation and in-depth investigation of placental exposure to environmental metals in maternal circulation. Central to this method is an in vitro analog of the maternal-fetal interface and its dynamic tissue-specific environment constructed using primary human placental cells grown in a microengineered device. Using cadmium as a representative toxicant, we demonstrate the proof-of-concept of emulating the human placental barrier subjected to the flow of cadmium-containing maternal blood to show how this model can be used to examine adverse biological responses and impaired tissue function on both the maternal and fetal sides. Moreover, we present a mechanistic study of maternal-to-fetal cadmium transport in this system to reveal that efflux membrane transporters expressed by trophoblasts may play an important protective role against cadmium-induced toxicity. Finally, we describe metabolomic analysis of our microphysiological system to demonstrate the feasibility of discovering metabolic biomarkers that may potentially be useful for detection and monitoring of cadmium-induced placental dysfunction.

Pregnancy is an essential process of reproduction in which the female body undergoes remarkable remodeling in a highly coordinated fashion to support the development of the fetus. By nature, most of the biological changes that occur during this process are transient, but altered maternal responses to external stimuli due to pregnancy can also result in lasting effects with long-term health consequences. As a representative example, pregnancy is known to increase maternal sensitivity and susceptibility to adverse effects of foreign substances, especially those encountered in settings of environmental or occupational exposures.

Among such materials are toxic metals, such as lead, mercury, cadmium, and arsenic, which represent a class of environmental contaminants that can enter the maternal circulation through dermal exposure, ingestion, or breathing. Using mass spectrometry-based methods, scientists have revealed the presence of these environmental metals in cord blood, placental tissues, and maternal blood during pregnancy. Epidemiology studies have shown that high-level exposures to these toxicants are associated with various pregnancy complications, including miscarriage, preterm birth, and low birth weight. Many of these ad-verse outcomes have also been linked to impaired childhood development, as well as the increased risk of cardio-vascular, neurological, and metabolic diseases in adult life.

Despite widespread recognition of prenatal metal exposure as an important public health concern, much remains to be learned about the biological basis and consequences of this problem. Advances in environmental toxicology in the last decades have generated a wealth of information and insight on how metals can enter and accumulate in the human body and how metal-specific free radicals produced by the toxicants may elicit abnormal biological responses, including oxidative stress and cellular injury, tissue inflammation, disrupted protein homeostasis, and sup-pressed activities of essential enzymes. However, it is unclear whether these general toxicological findings are fully applicable to predicting the specific risks and outcomes of toxic metal exposure during pregnancy-a period in which the human body undergoes extensive and dynamic anatomical and physiological changes to support fetal development.

In particular, one of the key outstanding questions in this area is how metals in the maternal system affect the placenta, which is one of the least understood organs in the human body uniquely associated with pregnancy. The placenta functions as the interface between the mother and the fetus that enables a controlled exchange of nutrients, oxygen, hormones, and other essential molecules, while also providing a restrictive barrier to prevent the passage of harmful substances from the maternal blood to fetal circulation. Importantly, contrary to this general notion of the placenta as a protective barrier, researchers have revealed measurable levels of mercury, lead, arsenic, and cadmium in various clinical specimens of human pregnancy, including amniotic fluid, cord blood, and placenta explants, showing the ability of metals of maternal origin to cross the placental barrier and for some metals, reach the fetal compartment. By demonstrating the potential of these materials as a threat to pregnancy, this body of work has provided a scientific basis for raising the poorly understood question of how toxic metals accumulating in fetal tissues affect the development of the fetus.

Equally significant gaps exist, however, in our knowledge about the effect of environmental metals on the maternal-fetal interface of the placenta that plays a vital role in the maintenance of pregnancy. Specifically, it remains elusive whether the presence and trafficking of toxic metals can directly harm cellular components of the placental barrier. Moreover, little is known about whether and how such exposures impair the transport function of the placental barrier, which may alter material exchange between the mother and the fetus to disrupt fetal development. Despite significant progress in clinical research on human pregnancy, investigating these questions directly in pregnant women continues to be a major challenge due to ethical constraints.

As an alternative, whole human placentas or placental villous explants can be used to develop ex vivo models for such studies. These models offer the advantage of preserving the anatomical and biological complexity of the native organ, but their broader applicability is often limited by the need for fresh clinical specimens and the technical challenges associated with reproducing and maintaining vascular perfusion in a controller manner, which is essential for studying placental function. Efforts to model placental toxicology using human cells instead have relied primarily on conventional culture of placental cell lines, which offer limited capacity to recapitulate the structural and functional complexity of the human placenta needed to adequately represent its in vivo responses to environ-mental metal exposure. Taken together, these limitations highlight the need for new experimental platforms that can complement existing preclinical models of the maternal-fetal interface in the human placenta.

As a step towards addressing this need, here we introduce a bioengineering approach for experimental modeling and in-depth preclinical studies of human placental exposure to environmental metals. This method is enabled by a microengineered system capable of allowing clinically sourced primary cells to generate vascularized human placental tissues that can be perfused in a controlled manner to recreate the maternal-fetal interface of the human placenta and its hemodynamic environment. We present an example, non-limiting case study in which cadmium was used as an environmentally relevant model toxicant to demonstrate how the bioengineered placental analog can be used to visualize and measure an array of biological responses and relevant functional endpoints to generate quantitative, high-content data useful for developing a more detailed understanding of metal toxicities. Given the crucial role of mem-brane transporters on the trophoblast layer of the placental barrier in regulating fetal exposure to xenobiotics, we also show how our model can be used to investigate efflux transporters-specifically breast cancer resistance protein (BCRP), one of the most abundant efflux transporters in the human placenta- and their protective role against metal toxicities. Moreover, we demonstrate the potential of our platform for biomarker discovery by presenting unbiased global metabolomic analysis of the maternal and fetal compartments to identify tissue-specific metabolites that can indicate placental dysfunction due to cadmium toxicity.

### Results

### Design of Bioengineered Human Placental Tissue

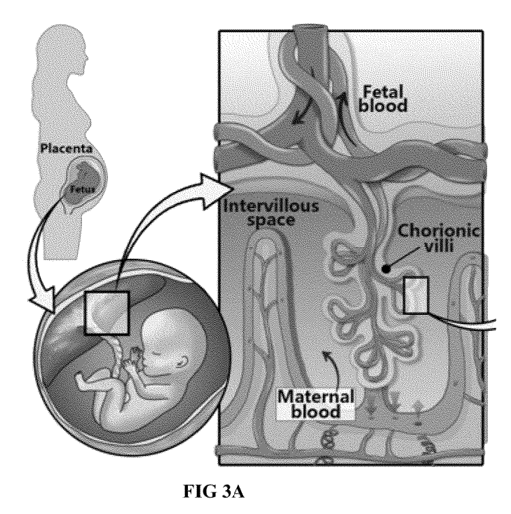

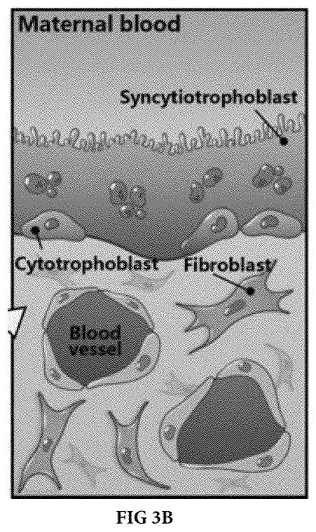

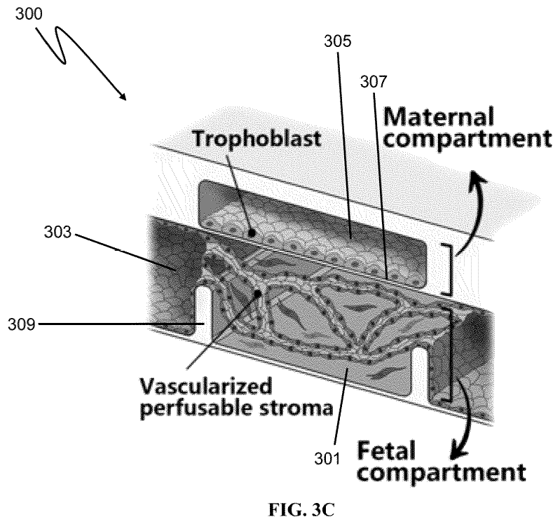

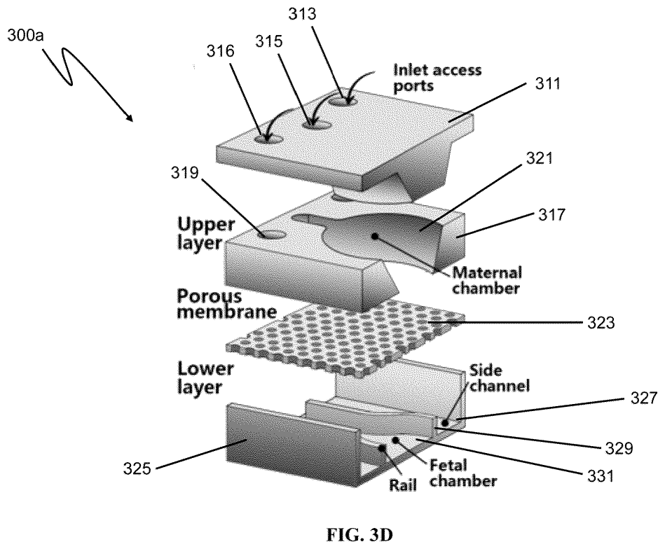

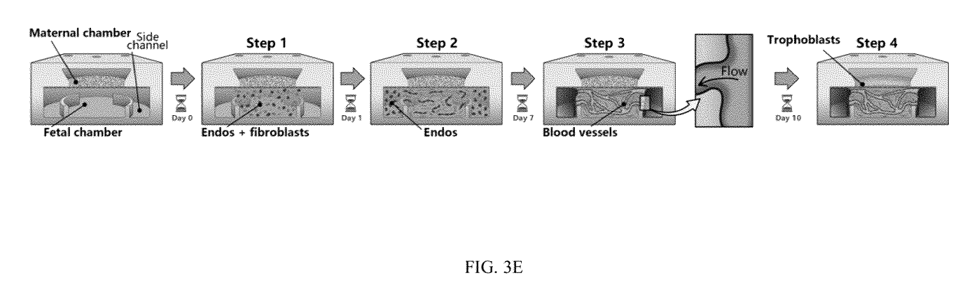

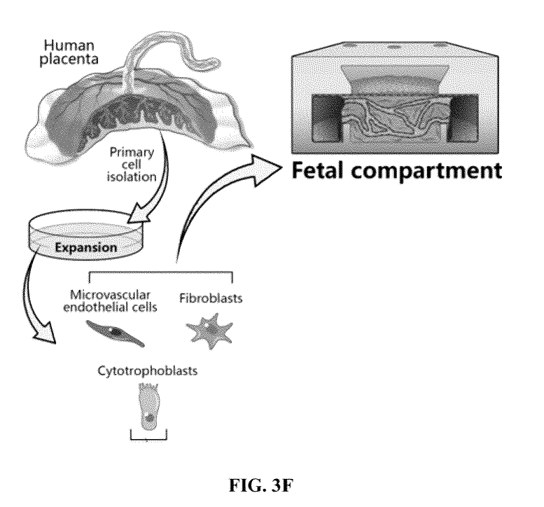

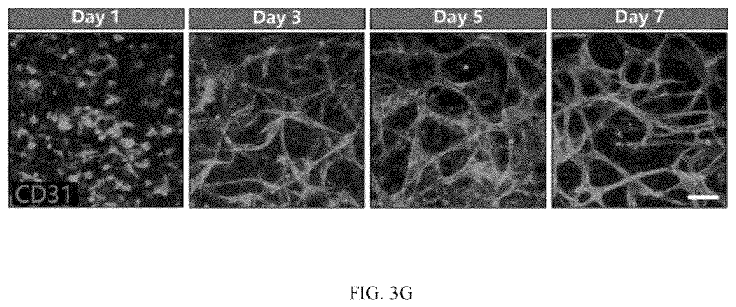

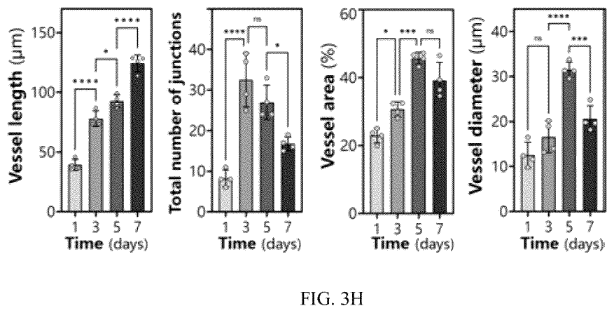

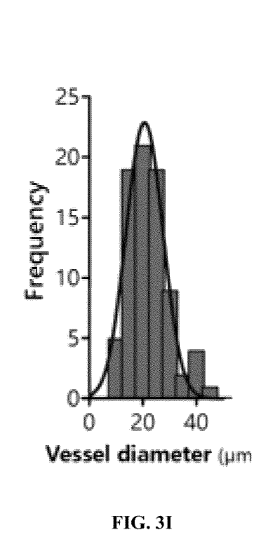

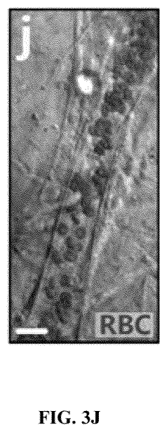

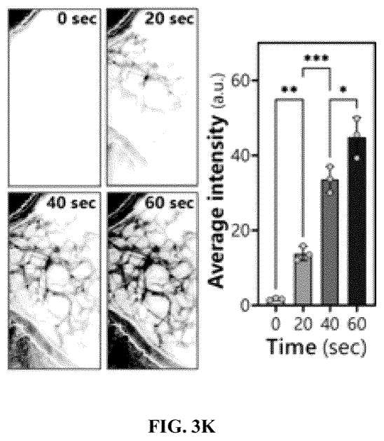

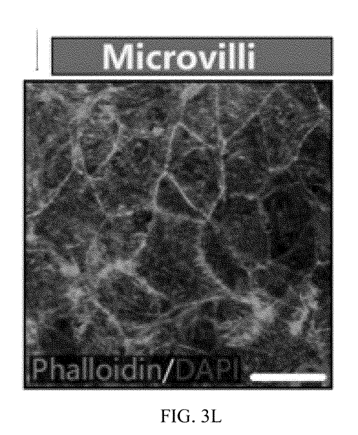

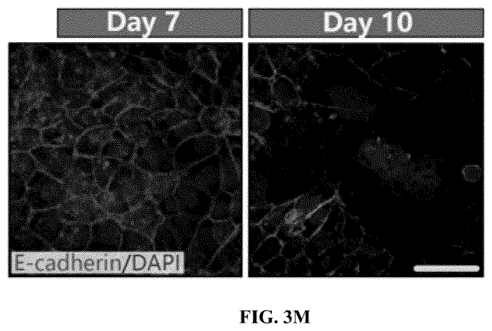

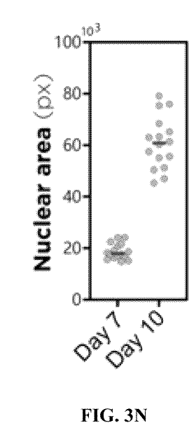

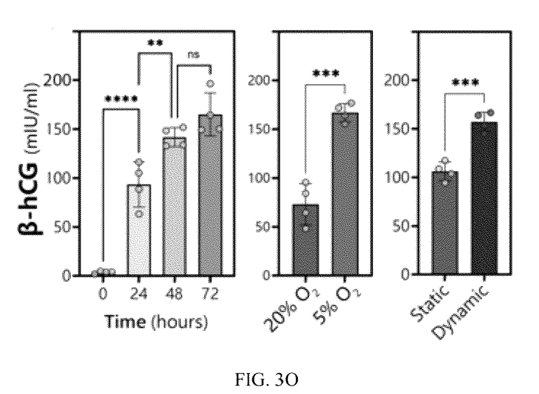

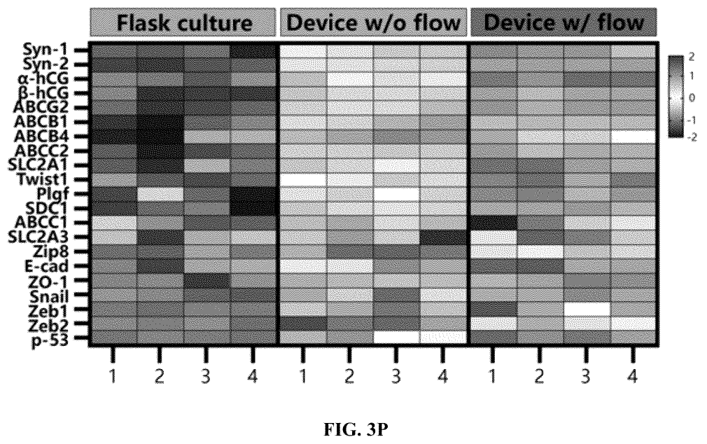

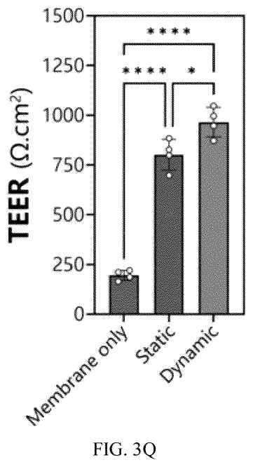

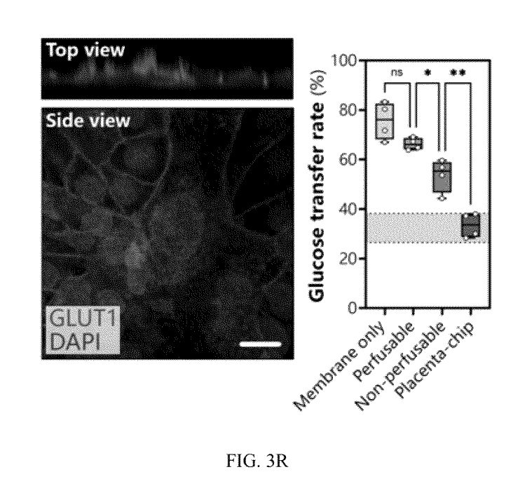

In the placenta, maternal-fetal interactions occur through molecular exchanges between the maternal blood pooling in the intervillous space and the fetal blood circulating in the vasculature of the chorionic villi (FIG. 3A). Physically separating these two compartments is a multilayered tissue assembly known as the placental barrier that consists of trophoblast cells facing the maternal blood and the underlying connective tissue containing a network of capillary blood vessels and stromal cells (FIG. 3B). Our bioengineered placental tissue was designed to capture the multicellularity and 3D structural organization of this functional unit of the placenta that forms the maternal-fetal interface (FIG. 3C).

An example, non-limiting model was built in a microfabricated device made of poly (dimethylsiloxane) (PDMS) that consists of i) an upper layer containing a 2-mm circular chamber, referred to as the maternal chamber, ii) a lower layer with a 2-mm circular chamber at the center, referred to as the fetal chamber, and iii) a thin semipermeable membrane with 1-μm pores sandwiched between the two overlapping layers (FIG. 3D). The lower layer of the device also contains micro-channels on either side of the fetal chamber, and these two compartments are separated by a micropatterned rail structure protruding from the floor of the device (FIG. 3D). Each chamber is connected to its own set of access ports contained in another substrate bonded to the upper layer to permit independent control of cells and their fluidic environment, as well as sampling of device effluent in a compartment-specific manner (FIG. 3D).

The first step of model construction in this device is to generate the fetal compartment of the placental barrier containing a vascularized connective tissue that supports fetal circulation (FIG. 3C). This is accomplished by injecting an extracellular matrix (ECM) hydrogel precursor mixed with human placental endothelial cells and fibroblasts into the fetal chamber and induce gelation to form a cell-laden hydrogel construct (Step 1 in FIG. 3E). Next, another batch of placental endothelial cells is seeded into the side channels adjacent to the fetal chamber and grown on the channel surfaces (Step 2 in FIG. 3E). In this configuration, the endothelial cells cultured in the hydrogel undergo a dynamic process reminiscent of vasculogenesis in vivo in which they become elongated and connect with one another to self-assemble into a 3D network of open blood vessels (Step 3 in FIG. 3E). During this process, the endothelial lining of the side channels sprouts into the hydrogel and anastomoses with the 3D vessels, making the self-assembled vascular network accessible and perfusable from the side channels. Finally, the maternal compartment of the placental barrier—which includes the trophoblast layer and the intervillous space containing maternal blood (FIG. 3C)-is recreated in this model by seeding human trophoblast cells into the maternal chamber and growing them on the upper side of the intervening membrane to form a confluent monolayer (Step 4 in FIG. 3E).

### Production and Characterization of Bioengineered Human Placental Tissue

To model the human maternal-fetal interface with high biological fidelity, we used primary cytotrophoblasts, villous microvascular endothelial cells, and fibroblasts isolated from term human placentas after delivery (FIG. 3F). Importantly, given the sensitivity of primary human placental cells to oxygen, these cells were maintained at 5% oxygen, which approximates physiological oxygen levels in the human placenta, throughout culture to allow them to preserve their native cellular phenotype.

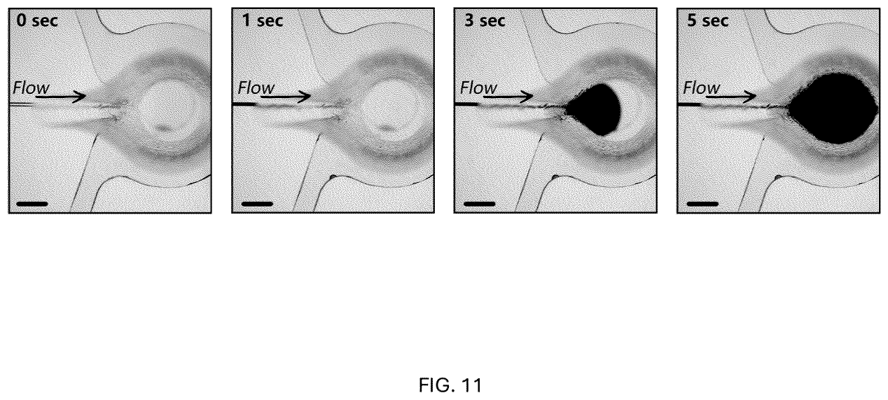

For tissue production in the device, we first prepared a mixture solution containing placental endothelial cells, fibroblasts, fibrinogen, and thrombin, and injected it into the fetal chamber. During this step, the solution was stably pinned along the microfabricated rails due to surface tension, and the advancing meniscus was guided by this structure until the entire chamber was filled (FIG. 11). This design permitted spatial confinement of the injected mixture in the fetal chamber without leakage to the side channels and the maternal chamber.

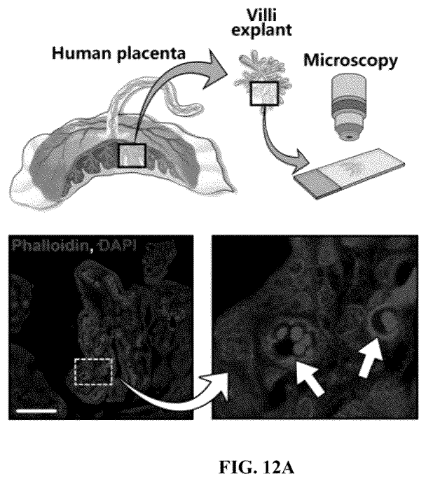

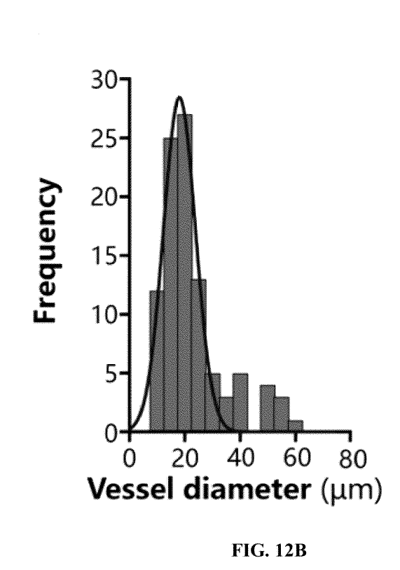

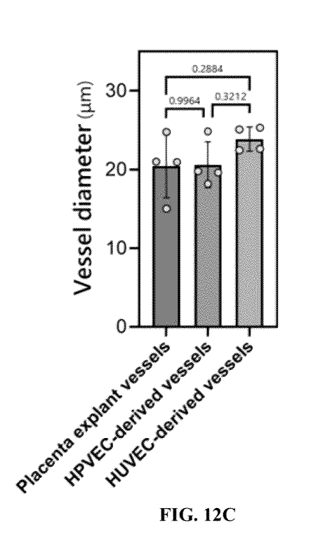

Fluorescence microscopy of the endothelial cells during 3D culture in the fibrin scaffold revealed rapid reorganization of individual cells into interconnected endothelial tubes, which was visible within 3-4 days of culture (FIG. 3G). The self-assembled blood vessels appeared to stabilize over time, yielding an average diameter of 19.9±7.6 μm by day 7 (FIGS. 3H, 3I). This value closely approximated the mean vessel diameter (20.3±11.7 μm) measured in human placental villi explants (FIGS. 12A, 12N). Of note, when the fetal vasculature was formed by human umbilical vein endothelial cells (HUVECs)-a non-tissue-specific endothelial cell type widely used to model the endothelium of blood vessels in microphysiological systems, the resulting vessels were larger, though not significantly so (23.9+1.5 μm) (FIG. 12C).

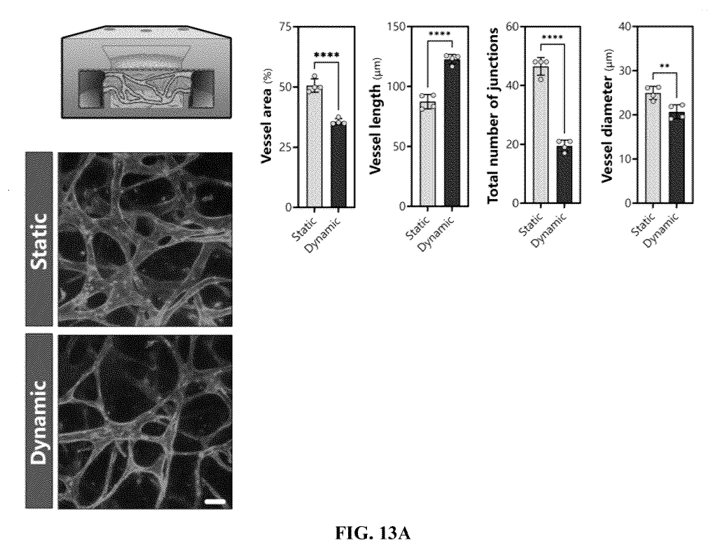

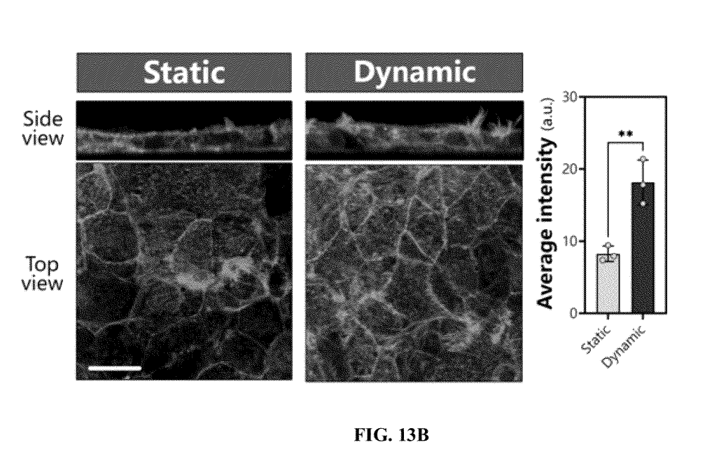

The vasculature also became perfusable within 7 days in this model (FIGS. 3J, 3K), after which the entire network was constantly perfused with media throughout the culture period. Interestingly, the blood vessels maintained in this flow condition appeared morphologically different from those in static conditions without vascular perfusion. The most notable difference was the smaller size of these vessels (FIG. 13), which might be viewed as a better representation of their microvascular origin. This observation was verified by quantitative comparison of vascular features that showed statistically significant reduction in vessel diameter, area, and the number of junctions in the dynamic culture condition with flow (FIG. 13).

In the upper half of the device, cytotrophoblasts were introduced into the maternal chamber on day 7 after the completion of vasculogenesis in the underlying fetal chamber. With the entire system maintained at 5% 02 while being perfused with media, these cells remained proliferative and continued to grow for 3-4 days to form a confluent monolayer covered with microvilli on the apical surface (FIG. 3L). Interestingly, the density of the microvilli was significantly lower when the trophoblasts were cultured in the absence of flow (FIG. 13B). Over the next 3 days, the trophoblast cells in the monolayer spontaneously fused and began to exhibit the morphological characteristics of the placental syncytium in vivo, which was evidenced by a loss of intercellular junctions and aggregation of cell nuclei (FIGS. 3M, 3N). This physiological differentiation of cytotrophoblasts into syncytiotrophoblasts was observed in the monolayer surface and further verified by a progressive increase in the level of β-human chorionic gonadotropin (β-hCG) in the effluent of the maternal chamber over time (FIG. 3O). Of note, our data revealed significantly higher levels of β-hCG due to the lower oxygen tension (5%) and flow conditions used in our model (FIG. 3O), illustrating the capacity of the physiologically relevant environment to help the cultured trophoblasts express more differentiated phenotype.

For further characterization of the syncytium, we then conducted RT-PCR to measure and compare the expression of placental cell markers. Analysis of the syncytialized cells isolated from our model showed marked upregulation of a set of genes with known associations with syncytiotrophoblast differentiation, such as syncytin, hCG, SLC2A1, and PLGF, when compared to trophoblasts cultured in flasks or our device under static conditions without flow for the same amount of time (FIG. 3P). In comparison to flask culture, our engineered placental model was also seen with decreased expression of intercellular junctional proteins (e.g., E-cad, ZO-1) and epithelial-mesenchymal transition markers such as Zeb2, whose expression levels are known to decrease with placental maturation. Similar differences were observed in the comparison of the same markers between flask culture and the engineered model without flow but in this case, the extent of changes, especially increases in the expression of syncytiotrophoblast differentiation markers, was not as substantial (FIG. 3P).

The beneficial effects of flow were also observed in the analysis of barrier function, which showed significantly higher levels of transepithelial electrical resistance between the maternal and fetal compartments under dynamic culture conditions with flow (FIG. 3Q). Expression of glucose transporter 1 (GLUT1)—the most abundant type of glucose transporters in the human placental barrier—was another functional marker measured in our analysis, which was shown by robust immunofluorescence staining along the apical surface of the trophoblast layer (FIG. 3R). Temporal measurement of glucose concentrations in the maternal and fetal perfusate yielded an average maternal-to-fetal transfer rate of 33.4%, which is within the physiological range of glucose transfer rates measured in perfused human placenta explants (FIG. 3R).

Modeling and analysis of cadmium toxicity in the human placenta

Next, we explored the utility of our bioengineered model for in-depth in vitro investigation of how environmental toxicants affect the maternal-fetal interface of the human placenta. This study focused on modeling placental toxicity of cadmium, an environmental metal monitored as a high-priority toxicant by environmental agencies due to its known or suspected adverse effects on human health. Cadmium is commonly found in the environment, and humans are exposed to this substance in a variety of situations, including food consumption, drinking water contamination, metal mining, fertilizer production, fossil fuel combustion, and tobacco smoking. Human exposure to cadmium occurs globally, but its prevalence has been particularly high in developing countries, which makes it an important global health equity issue.

Notably, cadmium is known to pose significant threats to pregnancy. According to a recent systematic review of epidemiological literature, for example, cadmium exposure increases the risk of low birth weight and preterm birth by 21% and 32%, respectively. Studies have also reported measurable levels of cadmium in human placentas and umbilical cord blood collected after delivery. Based on this evidence, it is suspected that cadmium accumulating in the placenta may exert direct or indirect deleterious effects on the maternal-fetal interface and its function, playing a causative role in the development of adverse pregnancy outcomes. The biological mechanism of how this process of placental dysregulation can occur, however, remains poorly understood. Although reproductive toxicity of cadmium has been investigated in various animal species human data are extremely limited, hampering our fundamental understanding of this problem. Therefore, our goal was to use our bioengineered tissue as a platform to model human placental exposure to cadmium, modulate its key parameters in a precisely controlled manner, and generate informative human-relevant data.

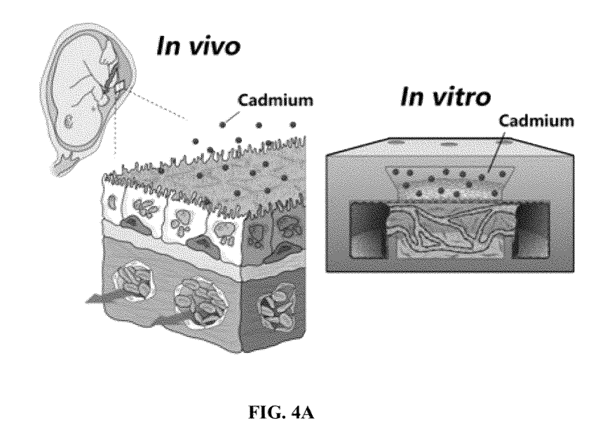

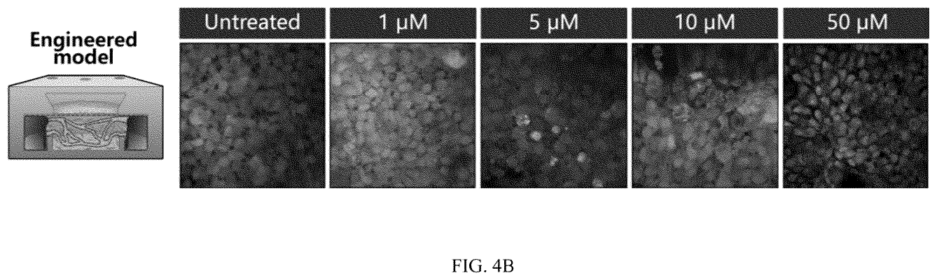

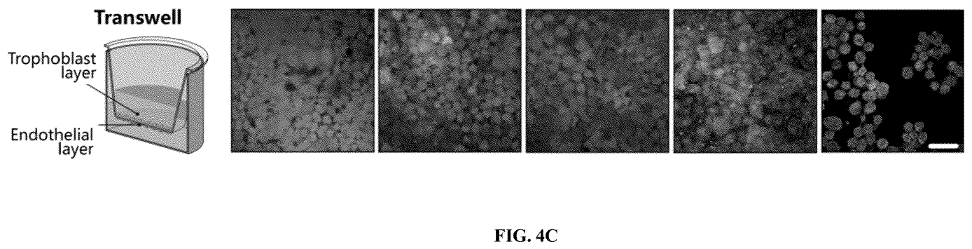

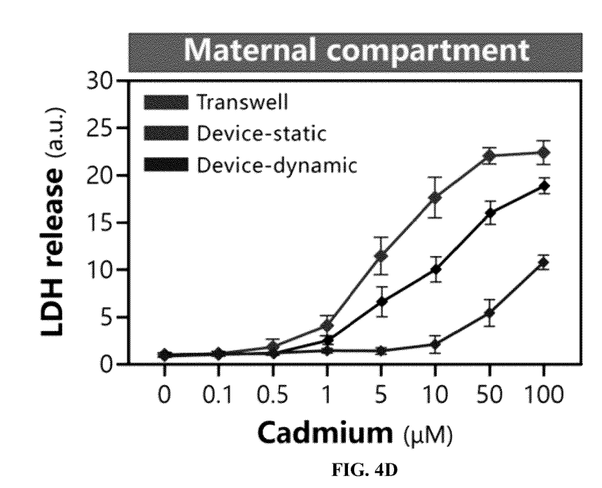

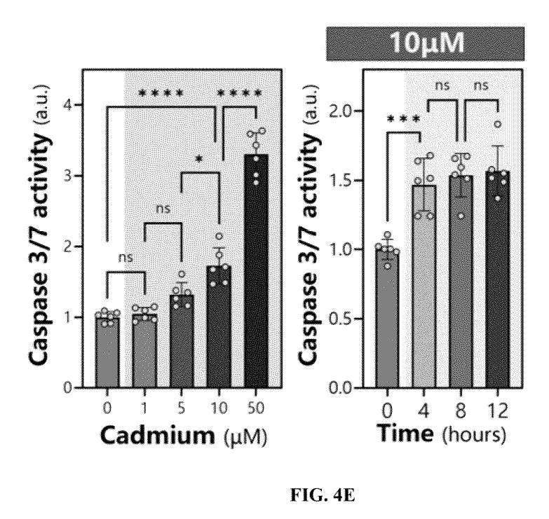

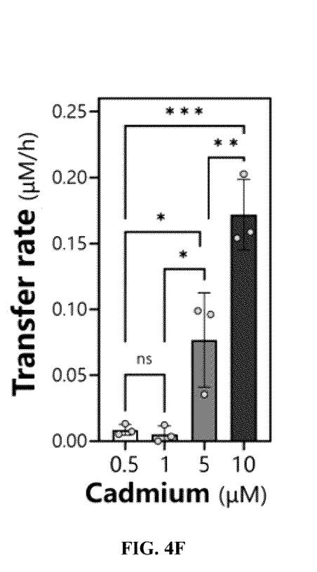

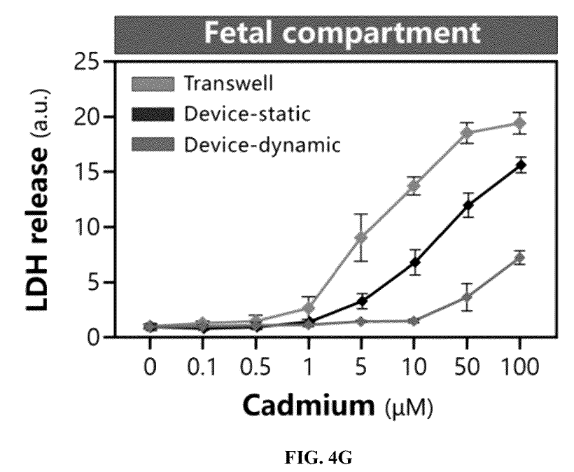

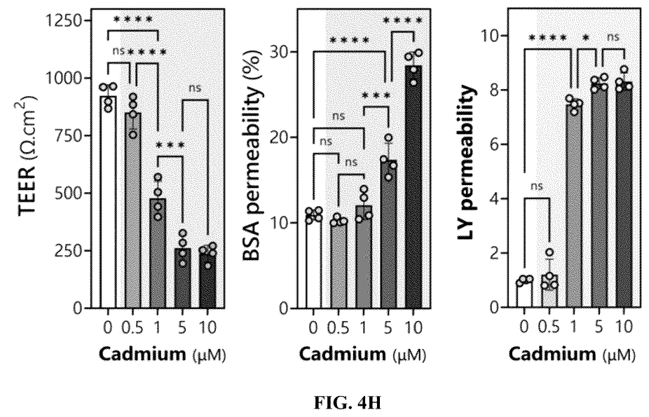

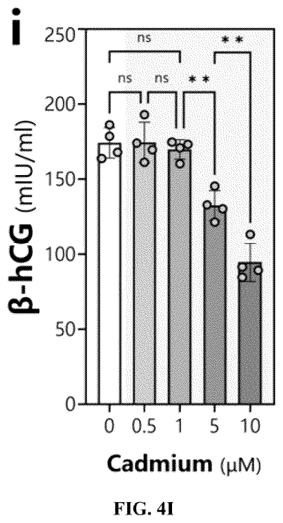

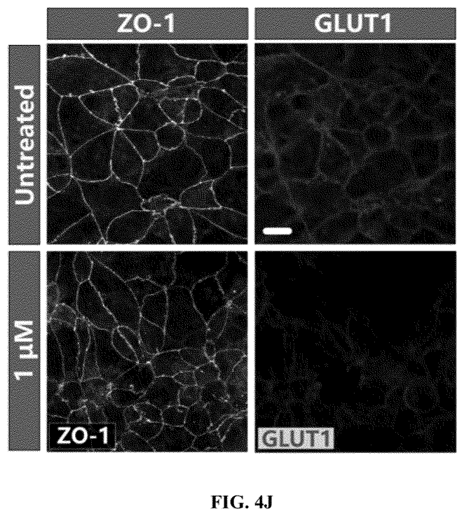

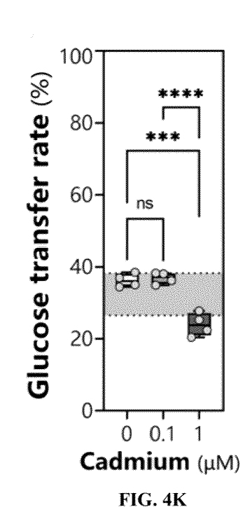

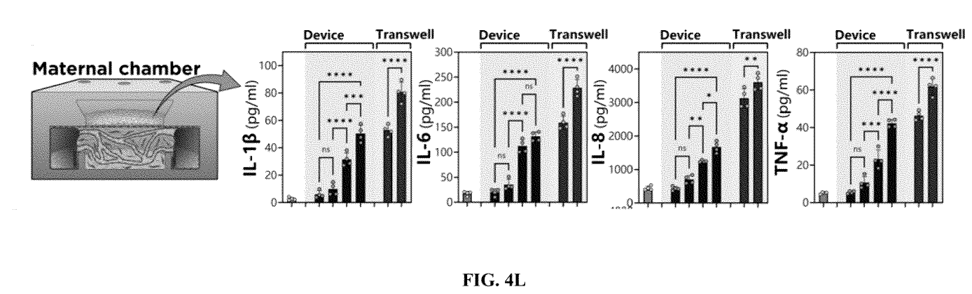

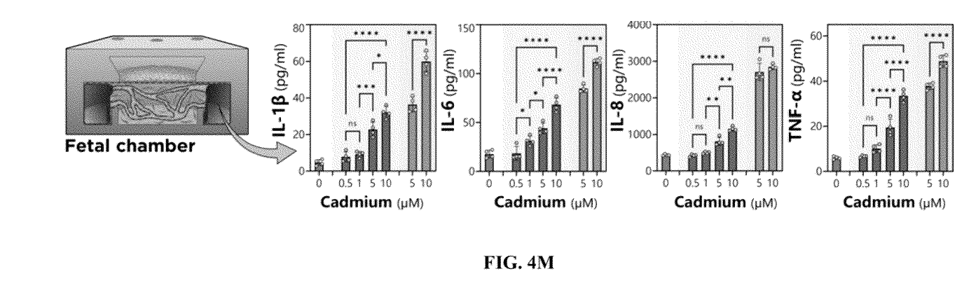

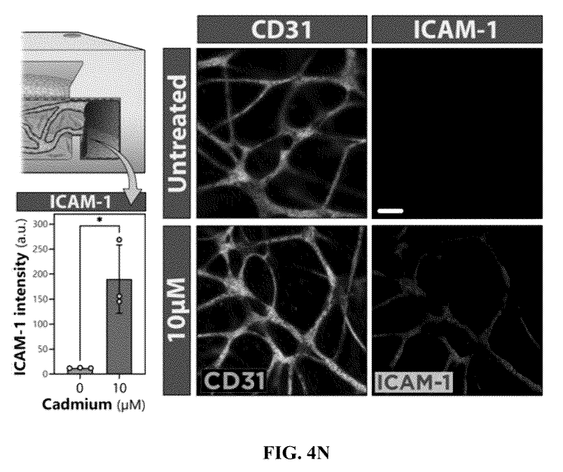

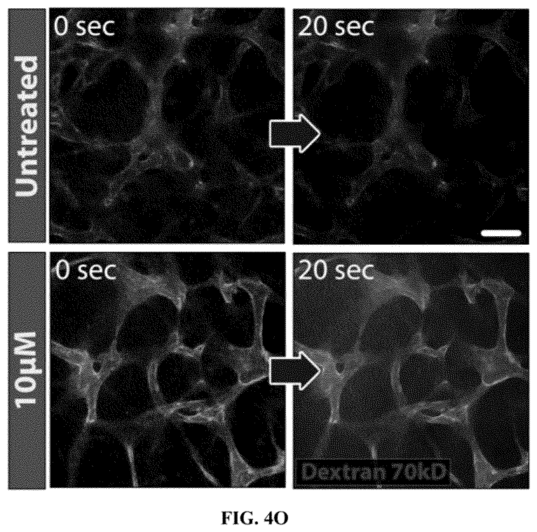

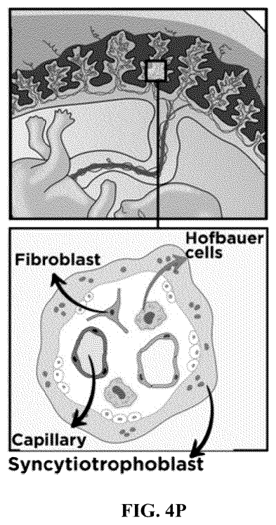

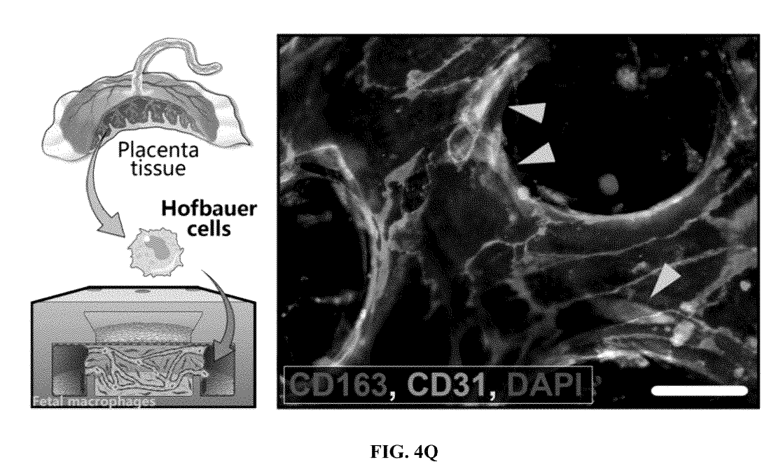

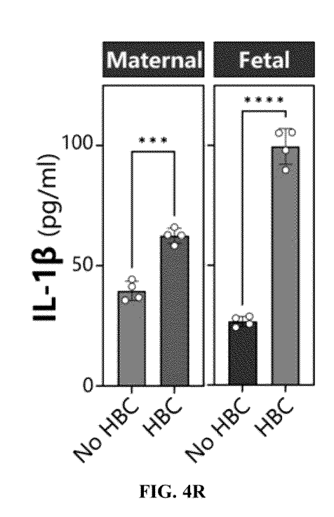

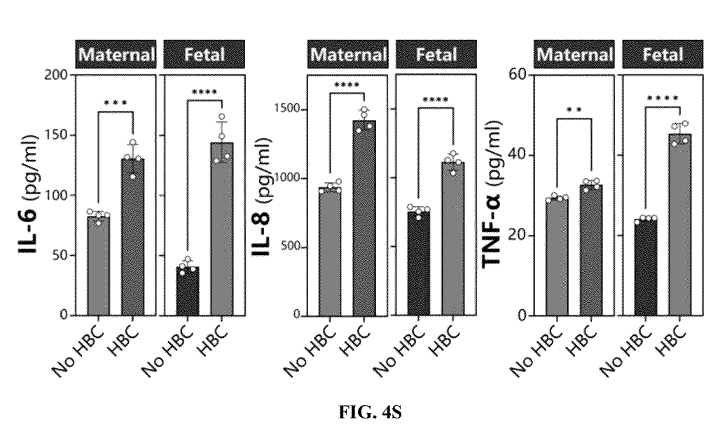

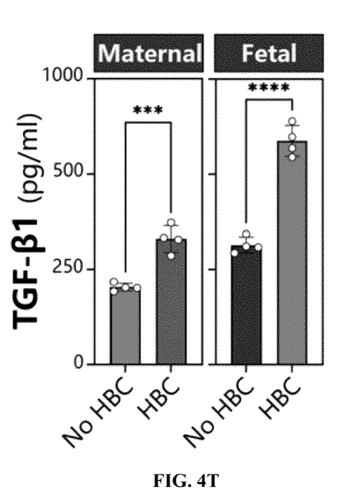

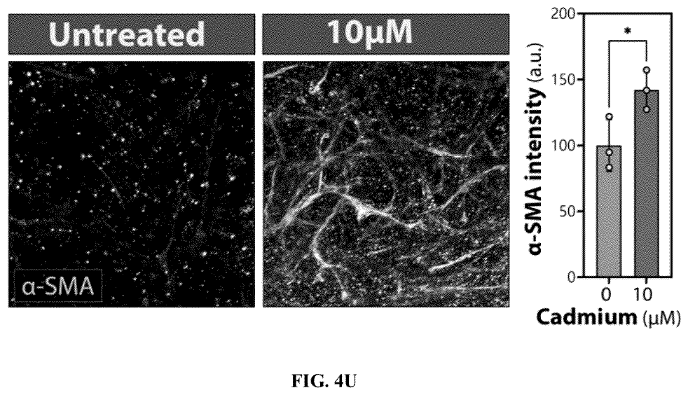

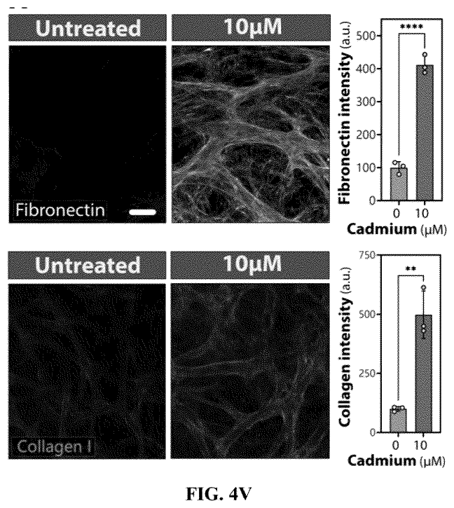

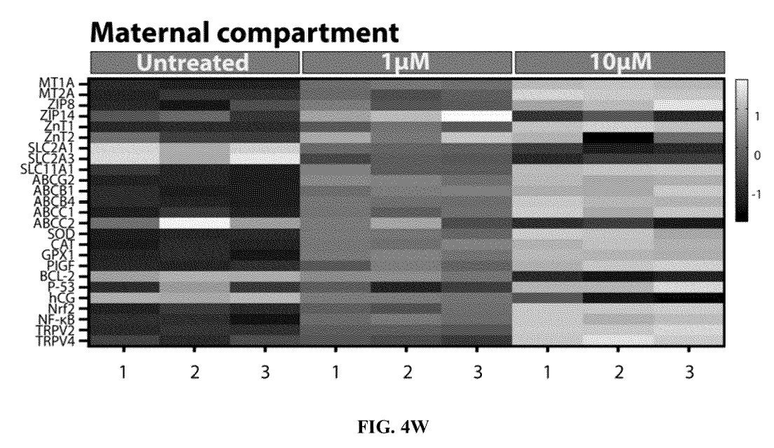

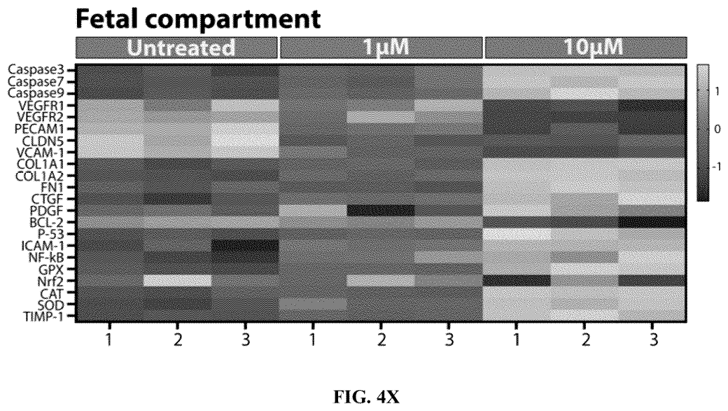

To this end, we established an exposure model (FIG. 4A) by adding cadmium chloride to the flow of trophoblast media in the maternal chamber. This model was first used to assess acute injury of the placental barrier after 24 hours of treatment in a range of cadmium chloride concentrations previously described in the literature. At lower doses ranging from 0.1 to 10 μM, which are more relevant to human situations, direct visualization of the trophoblast layer using live/dead staining showed intact or minimally affected cells, but treatment with higher concentrations (50-100 μM) resulted in larger numbers of membrane-compromised cells (FIG. 4B). Interestingly, at a given dose, the extent of this cellular injury was substantially greater in a Transwell-based exposure model constructed by growing the same batches of primary human trophoblasts and villous endothelial cells on either side of the Transwell membrane for the same duration (FIG. 4C), which was used to represent the gold standard model for in vitro studies of cadmium toxicity.

Consistent with these observations, the concentrations of lactate dehydrogenase (LDH) in the effluent of the maternal compartment exposed to lower doses of cadmium were statistically indistinguishable from those measured in the untreated group, and appreciable increases from the baseline levels were only detected at 10 μM or higher (FIG. 4D). By comparison, in the Transwell model and the engineered tissue maintained under static conditions without flow, the maternal LDH levels began to increase substantially even at 1 μM and were found to be more than twice as high as those in the dynamic model with flow at any given cadmium concentration greater than 1 μM (FIG. 4D). Our data also revealed significant upregulation of caspase activities in the trophoblast layer within a few hours of treatment at 10 and 50 μM of cadmium (FIG. 4E), indicating the potential of high-level cadmium exposure to induce rapid activation of apoptosis signaling in the placental syncytium.

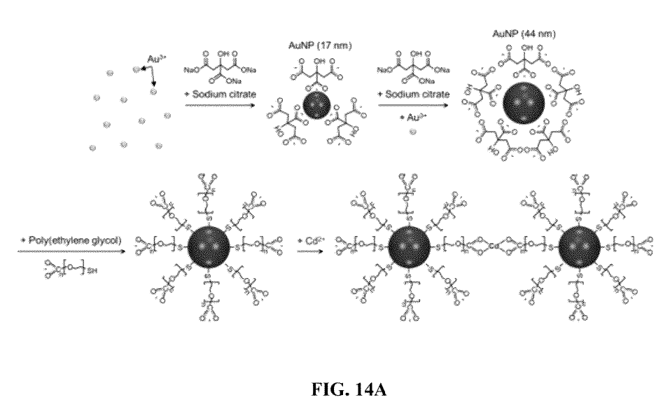

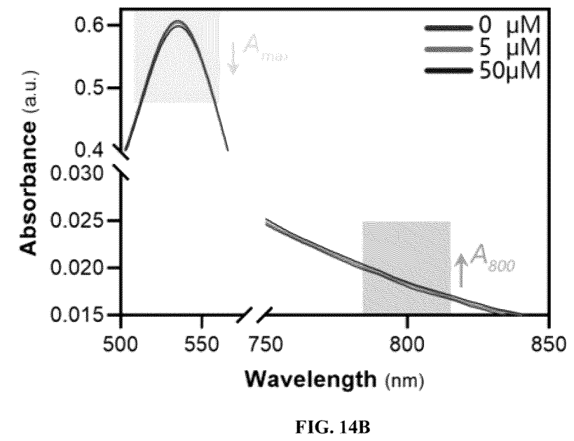

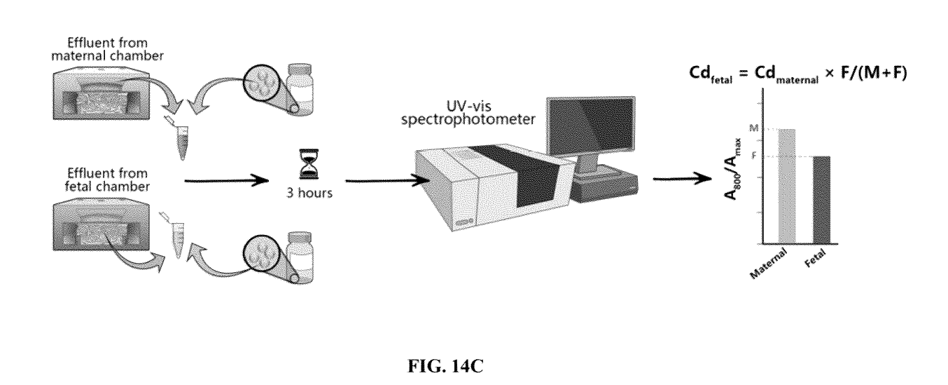

The observation of this concentration-dependent cytotoxicity in the maternal compartment led us to examine the effects of maternal cadmium on the fetal chamber side of our model. For this study, we first analyzed maternal-to-fetal transfer of cadmium by collecting device effluent from both the maternal and fetal chambers and measuring cadmium concentrations in the collected samples using nanoengineered plasmonic sensors designed specifically for cadmium detection and quantification (FIG. 14). In all conditions tested, this analysis revealed the presence of cadmium in the fetal compartment within 24 hours of exposure, but the rate of transfer was dependent upon the cadmium level on the maternal chamber side. At a cadmium concentration of 1 μM, the transfer rate was 0.004 μM/h, consistent with limited maternal-to-fetal transfer of cadmium in human pregnancies (FIG. 4F). At 10 μM, however, approximately 47.3% of maternal cadmium was transferred to the fetal compartment at a rate of 0.17 μM/h, likely due to cadmium-induced toxicity in trophoblasts. Notably, cadmium in the fetal compartment was found to induce LDH release from the vascularized stroma in concentration-dependent manners similar to those observed on the maternal chamber side, but the threshold level of cadmium at which tissue injury became evident in our dynamic model was higher (50 μM) (FIG. 4G).

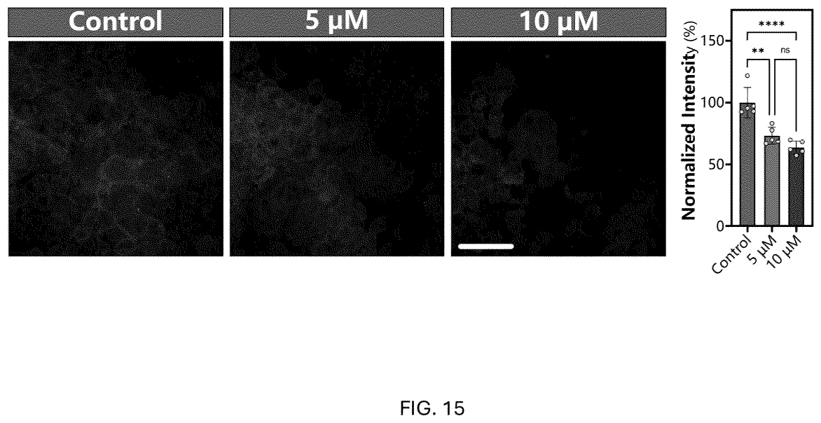

As expected, these cytotoxic effects produced by higher levels of cadmium also had a negative impact on the functional capacity of the engineered placental barrier. For example, at 10 μM, our exposure model showed compromised barrier function as demonstrated by significantly reduced electrical resistance across the barrier and its increased permeability to indicator dye molecules (FIG. 4H). What was unexpected, however, was substantially increased barrier permeability even at 5 μM (FIG. 4H), which was not associated with elevated LDH secretion and caspase signaling (FIGS. 4D, 4E, 4G). Consistent with these results, immunofluorescence analysis showed significantly reduced expression of E-cadherin by trophoblasts at 5 μM and higher concentrations (FIG. 15). Cadmium exposure at 5 μM also resulted in significant decreases in the production of β-hCG (FIG. 4I), suggesting that lower, non-cytotoxic levels of cadmium may still generate adverse effects on the placental barrier.

We then asked whether and how cadmium exposure affects the essential physiological function of the placental barrier as a mediator of maternal-to-fetal glucose transfer. Since our results showed compromised, leaky barrier at 5 μM and higher, we tested lower cadmium concentrations between 0 and 1 μM. When treated with 0.1 or 1 μM for 24 hours, the syncytialized trophoblast layer remained structurally intact and did not show any signs of a loss of barrier integrity (e.g., intercellular gaps). At 1 μM, however, the cells were seen with noticeable changes in the expression of glucose transporters, which was best characterized by decreased surface area of the barrier stained positive for GLUT1 (FIG. 4J). Supporting these results, the measurement of glucose levels in the maternal and fetal compartments revealed significantly decreased glucose transfer rates, which fell below the physiological range (FIG. 4K). This adverse response was not observed when the model was exposed to 0.1 μM.

### Cadmium-Induced Inflammation and Disruption of Placental Signaling

Clinical studies of prematurity have shown that inflammation in the placenta is associated with several clinical markers of fetal growth restriction and may play an important role in preterm birth⁴². Based on this clinical evidence and the known association of prenatal cadmium exposure with low birth weight and preterm birth, we reasoned that cadmium in the maternal blood may induce inflammation of the placental barrier and that this adverse response may be one of the ways in which cadmium exposure can disrupt placental signaling that may eventually lead to adverse pregnancy outcomes reported in the literature.

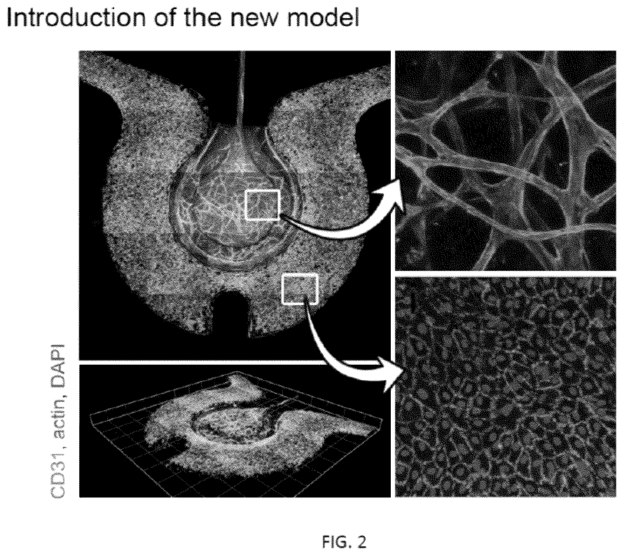

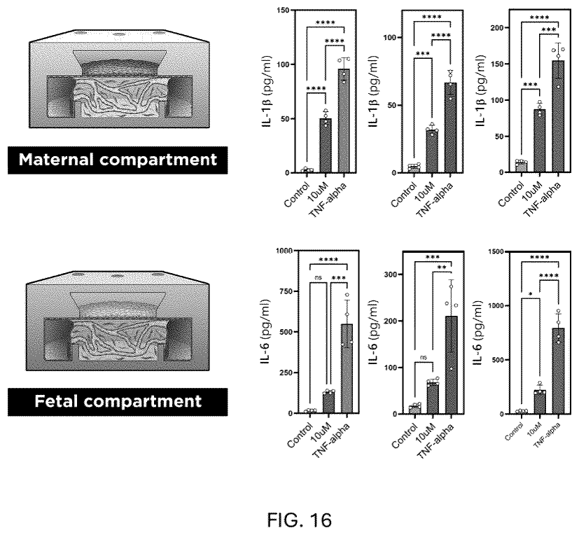

To investigate relevant biological responses in our exposure model, we measured the levels of four representative pro-inflammatory cytokines, including interleukin-8 (IL-8), IL-6, IL-1β, and tumor necrosis factor-alpha (TNF-α), all of which have been implicated in human placental inflammation. Analysis of maternal effluent following 24 hours of exposure showed dose-dependent inflammatory responses in which all four cytokines were produced in significantly increased amounts when the model was subjected to 5 μM or higher (FIG. 4A, FIG. 16), which coincided with the threshold above which barrier function and β-hCG production were compromised (FIGS. 4H, 4I). Treatment with lower doses (0.5, 1 μM) yielded no differences in the cytokine levels (FIG. 4L). Similar to our observation of platform-dependent susceptibility to cadmium-induced tissue damage (FIGS. 2B-2D), the levels of the same cytokines were 1.5-3-fold higher in the Transwell model (FIG. 4L).

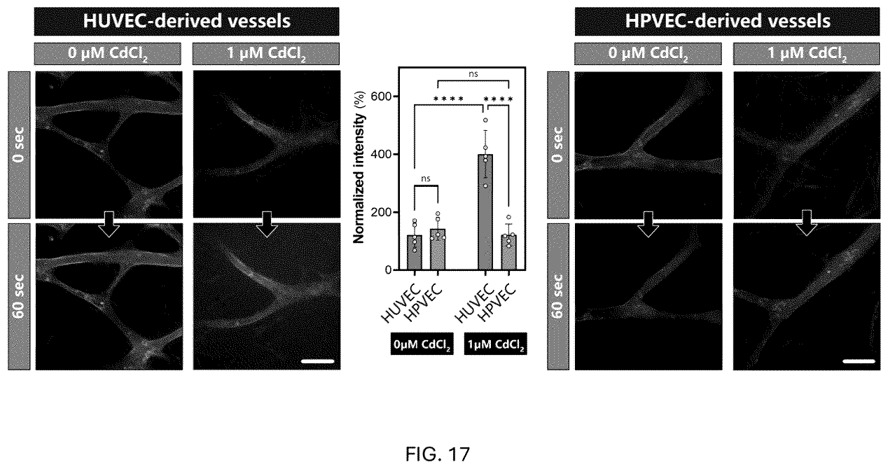

For each cytokine, measurements taken from the effluent of the fetal compartment of our model showed the same concentration-dependent trends, as well as lower cytokine levels than the Transwell control (FIG. 4M). The pro-inflammatory microenvironment of the fetal chamber resulting from higher-level cadmium exposure also led to the activation of the vasculature in the stroma as illustrated by increased endothelial expression of intercellular adhesion molecule-1 (ICAM-1) at 10 μM (FIG. 4N). The inflamed blood vessels appeared to retain their architecture but were found to be leakier during perfusion with 70 kDa Rhodamine B-dextran (FIG. 4O). Interestingly, this deleterious response demonstrating compromised endothelial barrier function due to cadmium exposure was observed even at 1 μM when the fetal blood vessels were derived from HUVECs instead of placental villous endothelial cells (FIG. 17), suggesting tissue-specificity of endothelial cells as an important factor in the susceptibility of the fetal tissue to vascular dysfunction in our model.

To more faithfully model the inflammatory milieu of the cadmium-exposed placental barrier, we then obtained donor-matched primary human Hofbauer cells, which are placental macrophages of fetal origin that reside in the villous stroma (FIG. 4P), and incorporated them into the fetal compartment of our system (FIG. 4Q). During tissue production, these cells were found mainly in the vicinity of the self-assembled blood vessels (FIG. 4Q). Notably, when the Hofbauer cell-containing model was treated with maternal cadmium at 10 μM for 24 hours, the production of IL-1B in the effluent of the maternal and fetal chambers was increased by 1.58 and 3.75 folds, respectively, as compared to control without Hofbauer cells (FIG. 4R). Similarly significant increases were observed in the analysis of the other cytokines (FIG. 4H), demonstrating the pro-inflammatory phenotype of Hofbauer cells previously suggested by histological studies of human placentas with inflammatory conditions (e.g., villitis).

Interestingly, inclusion of Hofbauer cells in the stromal compartment also resulted in substantially elevated levels of transforming growth factor-beta (TGF-β1) produced by cadmium exposure (FIG. 4T). Given previous studies showing the association of activated TGF-β1 signaling with fibrosis in preeclamptic human placentas, this result prompted us to examine the fibroblast population in our exposure model.

Immunofluorescence analysis of alpha-smooth muscle actin (α-SMA) revealed that the level of placental fibroblast activation was significantly increased as a results of treatment with 10 μM of cadmium chloride (FIG. 4U). Further supporting this observation, the stroma of the exposed model was also seen with profibrotic matrix remodeling as shown by increased deposition of fibronectin and type I collagen (FIG. 4V), suggesting the capacity of cadmium to induce fibrogenic responses in the placental barrier.

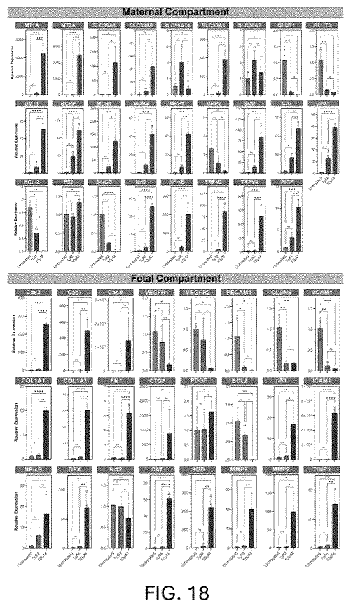

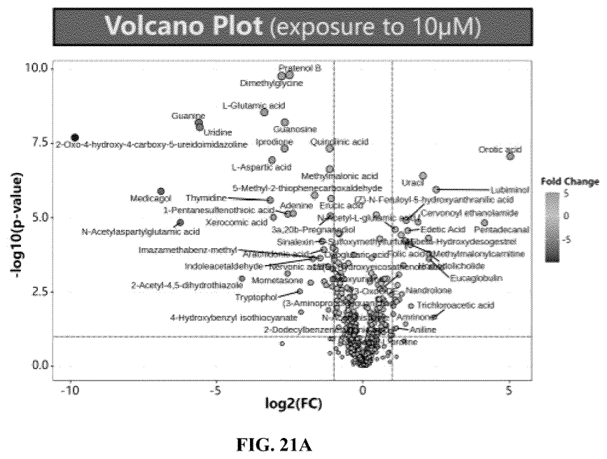

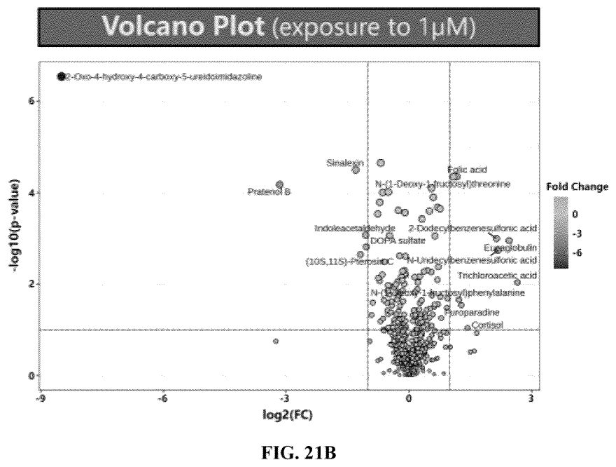

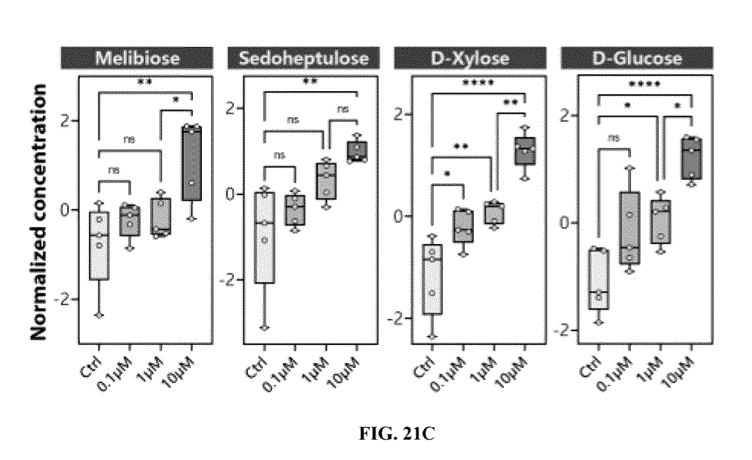

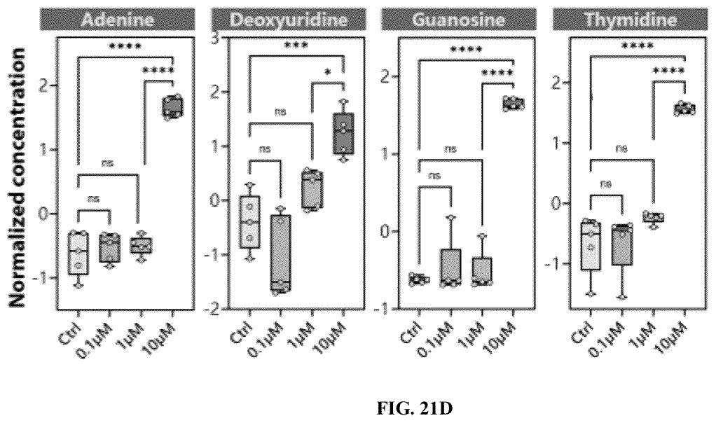

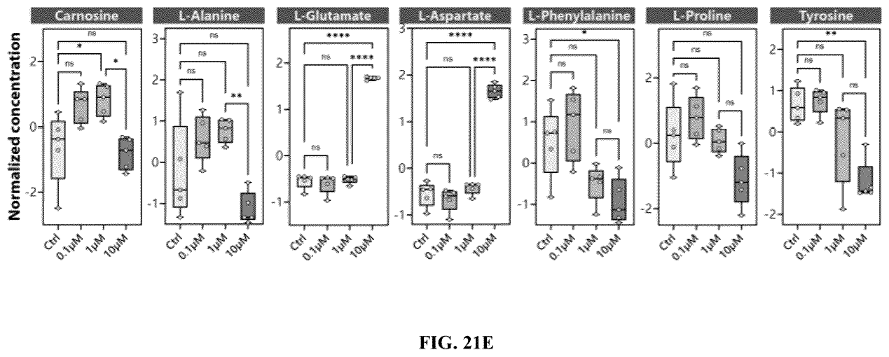

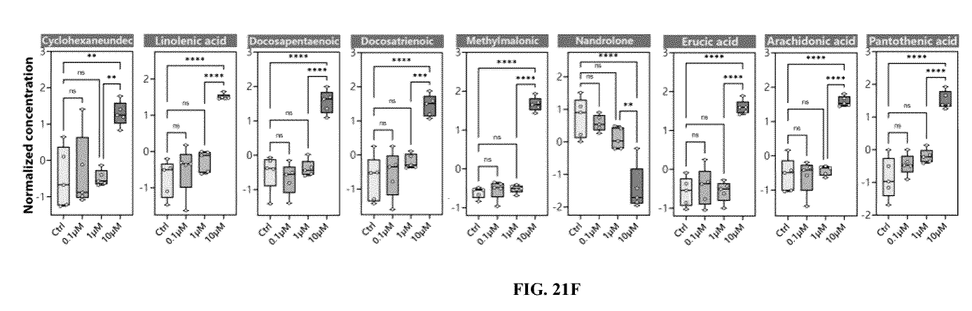

Finally, we performed RT-PCR analysis of our model with the goal of gaining further insight into how placental signaling may be altered or dysregulated by cadmium exposure (FIGS. 4W, 4X, FIG. 18). Examination of top 25 differentially regulated genes in the maternal compartment showed increased activities of biological signaling involved in cellular responses to oxidative stress, which was evident from significantly elevated expression of metallothionein (MT), glutathione perioxidase-1 (GPX1), superoxide dismutase (SOD), catalase (CAT), and transcription factor nuclear factor erythroid 2-related factor 2 (Nrf2) (FIG. RW). NF-κB was another gene related to oxidative stress and inflammation that was upregulated in the cadmium-treated model (FIG. 4W). Data also revealed signaling changes indicative of disrupted placental function, including dose-dependent reduction in the expression of hCG, as well as solute-carrier (SLC) genes, such as SLC2A1 and SLC2A3, that are responsible for producing glucose transporters (FIG. 4W). This result was in agreement with the findings of hCG ELISA (FIG. 21) and immunofluorescence of GLUT1 (FIG. 2J). Finally, cadmium exposure increased the expression of genes, such as ABCG2, ABCB1, and ABCC1, that encode ATP-binding cassette (ABC) transporters, a large family of membrane proteins mostly found in the syncytiotrophoblast layer of the human placental barrier that use energy from ATP hydrolysis for active efflux transport to limit fetal exposure to xenobiotics, drugs, and environmental toxins (FIG. 4W).

PCR analysis of cells collected from the fetal compartment of the cadmium-treated model showed increased signaling that mediates apoptosis (e.g., caspase-3, caspase-7) and antioxidant responses (e.g., SOD, CAT, GPX, Nrf2, NF-κB) (FIG. 4M). Consistent with our observation of cadmium-induced vascular inflammation (FIG. 4C) and fibrogenesis in the villous stroma (FIGS. 4U, 4V), upregulated genes in the exposure model also included ICAM-1, COL1A1, COL1A2, FN1, and CTGF (FIG. 4X). Data indicated that cadmium treatment resulted in dose-dependent decreases in the expression of vascular endothelial growth factor receptor (VEGFR) genes involved in placental angiogenesis (FIG. 4X). Also included in this group of downregulated genes was claudin-5 (CLDN5) that plays an important role in maintaining placental barrier function, especially in regulating ion/electrolyte transport.

Notably, our analysis also identified a set of genes whose expression levels were significantly altered by cadmium exposure in both maternal and fetal compartments, including NF-κB, Nrf2, SOD, CAT, and GPX (FIGS. RW, 4X, FIG. 18). These genes, which share a common function in mediating antioxidant responses, exhibited dose-dependent upregulation in both compartments. This finding demonstrates oxidative stress as a shared consequence of cadmium exposure in the maternal and fetal tissues of our model, while also highlighting their intrinsic capacity to mount a protective response against such oxidative insults.

### Investigation of Placental Efflux Transporters

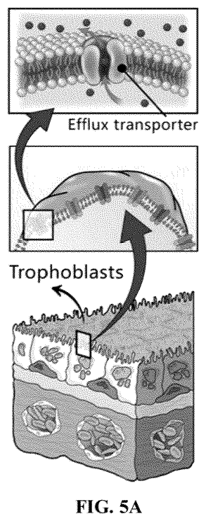

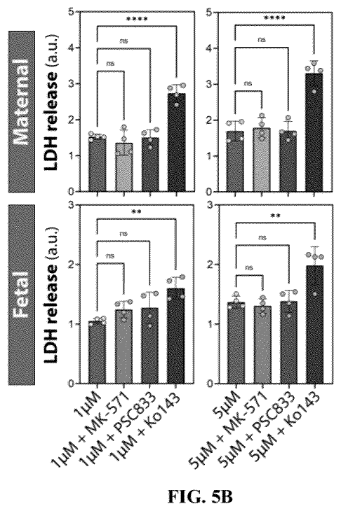

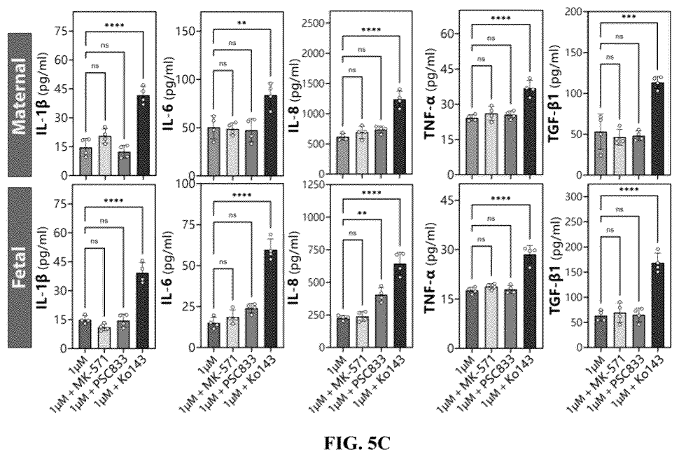

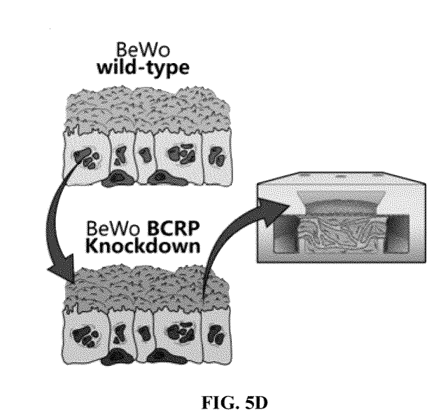

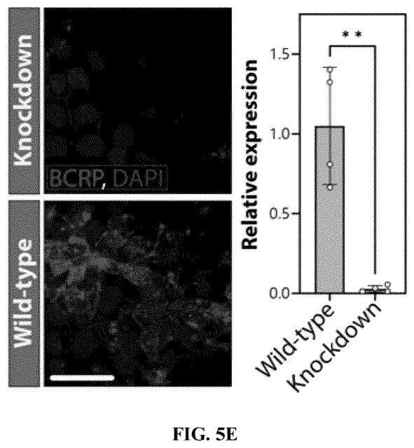

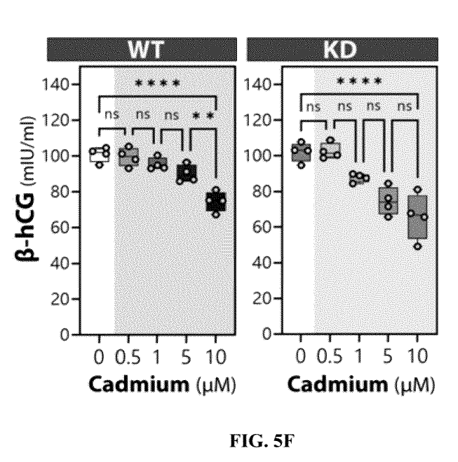

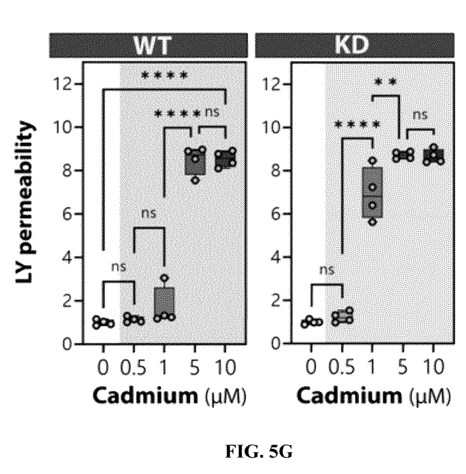

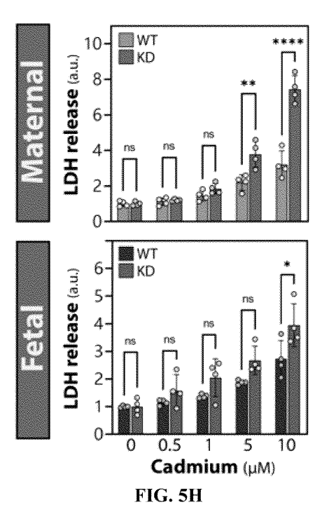

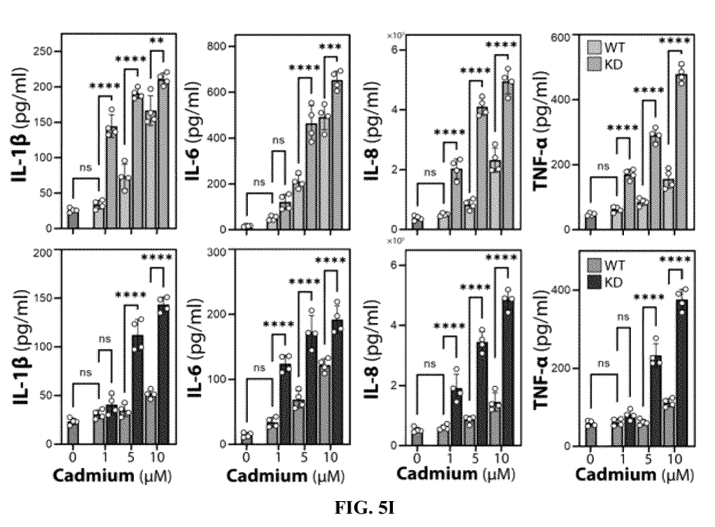

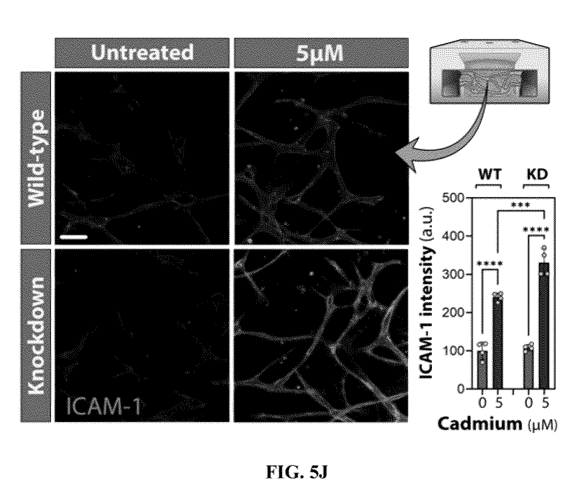

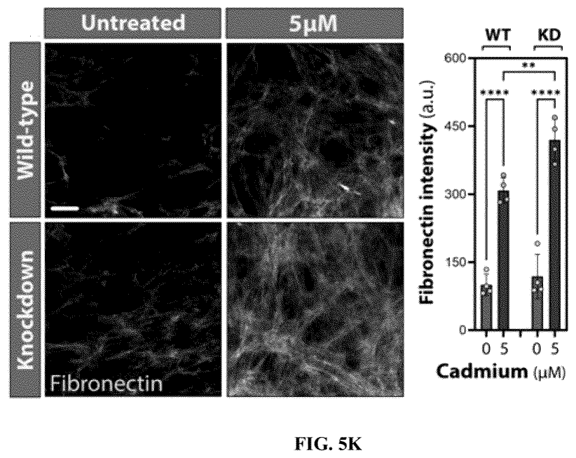

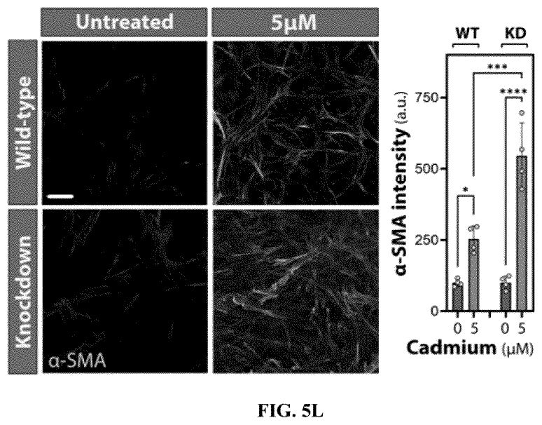

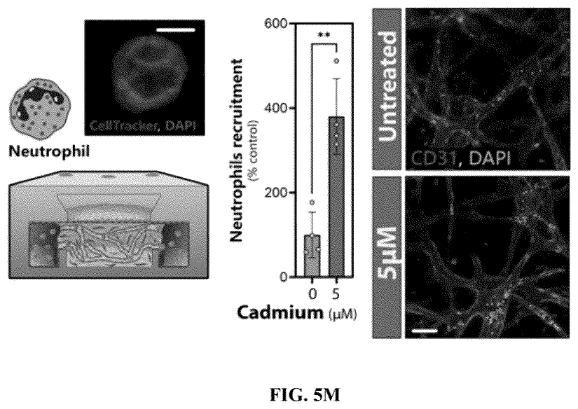

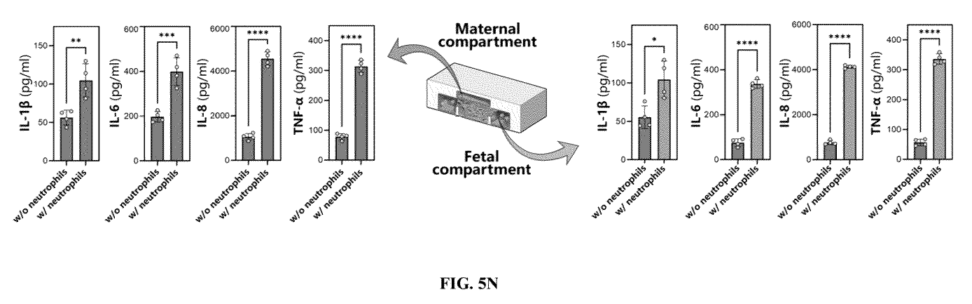

One of the findings of our PCR analysis that was particularly interesting was the increased expression of ABC genes in the trophoblast cells due to cadmium exposure (FIG. 4L). This subset included ABCG2, ABCB1, ABCB4, and ABCC1 that are known to encode breast cancer resistance protein (BCRP), multidrug resistance protein-1 (MDR1), MDR3, and multidrug resistance-associated protein 1 (MRP1), respectively. Notably, these are transmembrane proteins expressed by syncytiotrophoblasts in the human placenta that utilize ATP-derived energy to actively transport substances from the intracellular compartment to the extracellular space to prevent fetal exposure to xenobiotics (FIG. 5A). Although the general function of these proteins as efflux transporters has been established, their specific role and significance in placental toxicity of cadmium remain to be further investigated.

We next delved into how the detected efflux transporters affect responses of the bioengineered placental barrier to cadmium. Based on our PCR data, we hypothesized that during exposure, these proteins are upregulated to increase efflux transport of cadmium from trophoblasts and thus protect the barrier tissue against cadmium toxicity in our model. To test this hypothesis, we individually inhibited the activity of BCRP, MDR1, and MRP, which were selected as three representative efflux transporters, by adding their respective chemical inhibitors in the trophoblast media flowing in the maternal chamber and examined cadmium-induced cellular injury and inflammation in comparison to control without inhibition. In this study, we first treated the model with cadmium at 1 μM, a lower-level exposure condition that did not produce acute tissue injury as evaluated by live/dead staining (FIG. 4B) and LDH release (FIG. 4D). At this concentration, inhibition of MDR1 and MRP transporters did not change the level of LDH in the effluent of the maternal and fetal compartments (FIG. 5B). When the model was modified by using Ko143 to selectively inhibit BCRP function, however, LDH measurements showed nearly two-fold increases on the maternal chamber side, which occurred in the fetal compartment as well although to a lesser extent (FIG. 5B). This result demonstrating significantly increased deleterious effects of cadmium in the BCRP-inhibited model was also observed when the cadmium concentration was increased to 5 μM (FIG. B).

Analysis of cadmium-induced inflammation was performed by measuring pro-inflammatory cytokines in our model exposed to 1 μM of cadmium chloride. Although 5 μM was a threshold concentration necessary to trigger measurable cytokine production in the unmodified control group (FIG. 4L), we suspected that the inhibition of efflux transporters may have a significant impact even at lower cadmium levels that normally do not induce inflammatory responses. According to our data, suppressing the activity of MDR1 and MRP failed to alter the cytokine levels in the maternal chamber (FIG. 5C). By contrast, exposure to the same concentration of cadmium led to markedly elevated maternal production of all cytokines when BCRP was inhibited (FIG. 5C). This condition also resulted in significantly higher levels of all five cytokines in the fetal compartment as well (FIG. 5C).

Importantly, these data suggested that BCRP may play a critical role in protecting the maternal-fetal interface from tissue injury and inflammation during cadmium exposure. To validate this, we next used the small interference RNA (siRNA) technique to knock down trophoblast expression of BCRP in our model. Given the difficulty of applying this method to the primary culture of trophoblast cells, we used the BeWo b30 cell line to generate BCRP known-down cells, which were then cultured in the maternal chamber with primary placental fibroblasts and villous endothelial cells in the fetal compartment of the same device (FIG. 5D). siRNA did not affect the proliferative capacity and growth rate of these cells in our model, and they formed a confluent monolayer on the membrane surface within 1 day, after which the cells were stimulated with 50 μM forskolin for 3 days to induce syncytialization. The resultant placental syncytium expressed drastically reduced levels of BCRP compared to the control model derived from wildtype BeWo cells (FIG. 4E).

The engineered placental barrier was then treated with cadmium chloride at 0.5, 1, 5, and 10 μM. The effect of BCRP knockdown was first assessed by ELISA measurement of β-hCG, which appeared to show decreasing levels of production with the increasing dose of cadmium but without statistical significance, whereas hCG in the wild-type model was unaffected at the same levels of exposure (FIG. 5F). The threshold cadmium concentration at which the placental barrier began to show compromised integrity was lower in the knock-down model (1 μM) than was measured in the wild-type control (5 μM) (FIG. 5G). A loss of BCRP in the trophoblast cells also accentuated cytotoxic effects of cadmium at higher doses (1 and 5 μM) in both maternal and fetal compartments (FIG. 5H).

In these higher-level exposure conditions, the knockdown of BCRP resulted in significantly increased production of pro-inflammatory cytokines in the maternal and fetal compartments (FIG. 5I). Although the vascularized stroma in the fetal chamber of this model did not contain any modified cells, it exhibited higher levels of cadmium-induced vascular inflammation, fibroblast activation, and matrix deposition when compared to the wildtype control exposed to the same cadmium concentration (FIGS. 5J-5L), which is presumably due to elevated cytokine release in the maternal compartment. When the vasculature of this model was perfused with polymorphonuclear neutrophils, we observed large numbers of adherent neutrophils along the endothelial lining (FIG. 5m), reminiscent of neutrophil infiltration during fetal inflammatory response reported in pathological studies of the human placenta with acute inflammation. The capacity of the recruited neutrophils to exacerbate inflammatory responses to cadmium was demonstrated by the significantly higher levels of pro-inflammatory cytokines in these devices as compared to the control group without neutrophils (FIG. 5N).

Taken altogether, these data provide evidence that BCRP expressed by trophoblast cells, which is one of the most abundant ABC transporters in the human placenta, plays a central role in protecting the placental barrier from cadmium-induced acute tissue injury and inflammation in our model.

### Metabolomic Profiling of the Cadmium Exposure Model-Maternal Compartment

Traditionally, the primary goal of investigating the adverse effects of toxic environmental metals has been to assess the capacity and potential of the toxicants to induce tissue damage and immune reactions. Increasing evidence shows, however, that environmental metal exposure can also disrupt glucose, lipid, and hormonal homeostasis to elicit aberrant changes in metabolism. In fact, cadmium can cause metabolic dysfunction of the liver by altering the activity of the key enzymes of carbohydrate metabolism. Similar effects of cadmium have been demonstrated in other organs involved in metabolic function, such as the intestine and the pancreas. Unfortunately, this line of investigation has been scarce in the context of pregnancy, despite the fact that the placenta is a metabolically highly active organ responsible for supporting the growing fetus and itself. Recent epidemiology studies have examined the association of cadmium exposure with relevant conditions such as gestational diabetes, but it remains unknown whether and how cadmium can perturb and exert adverse effects on metabolic activities of the human placenta.

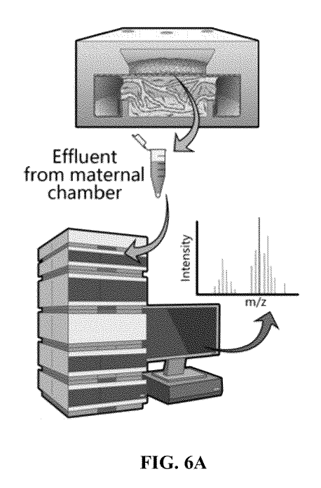

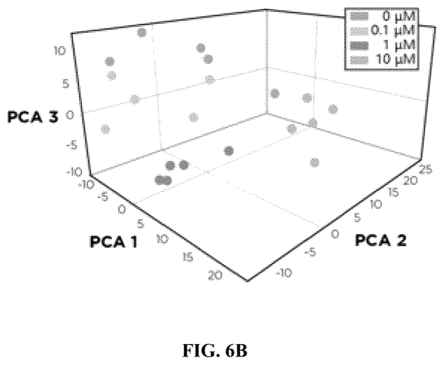

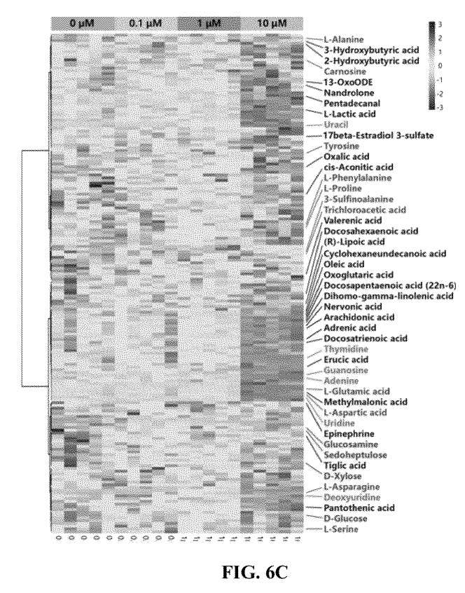

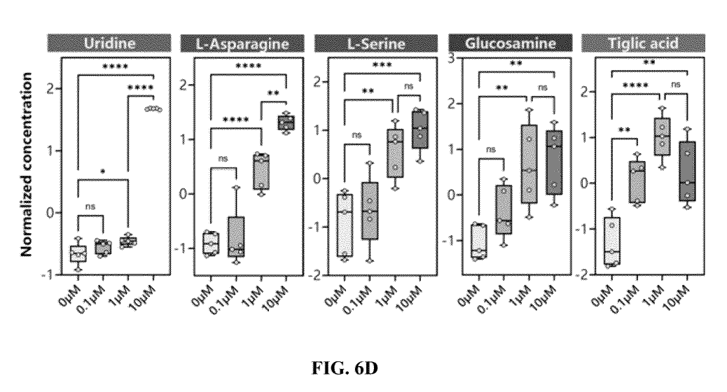

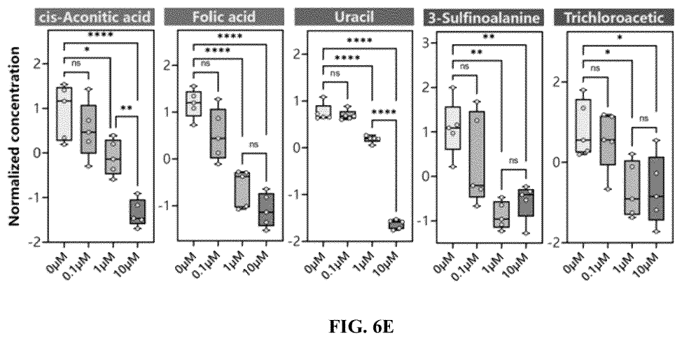

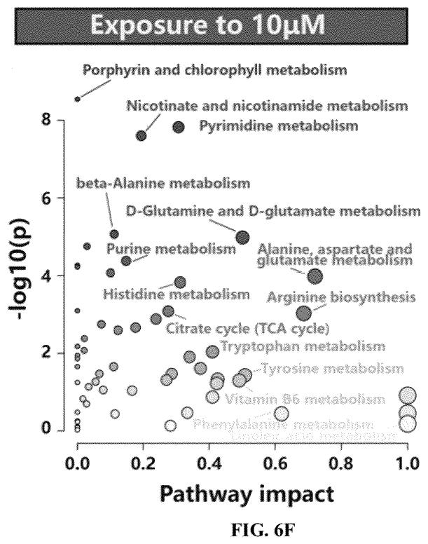

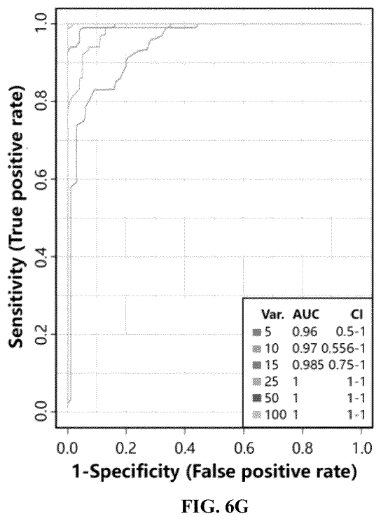

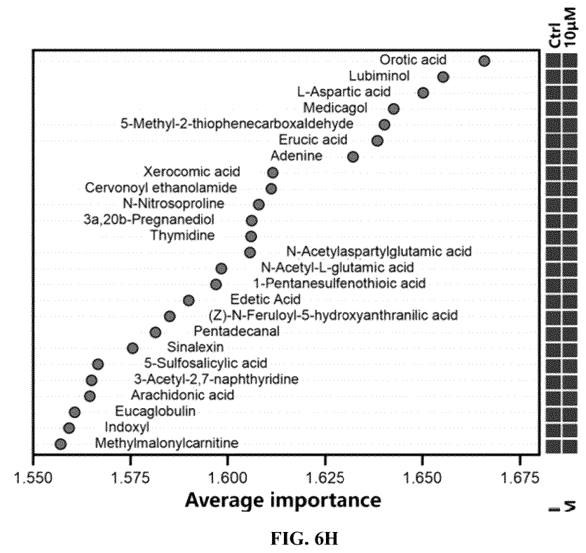

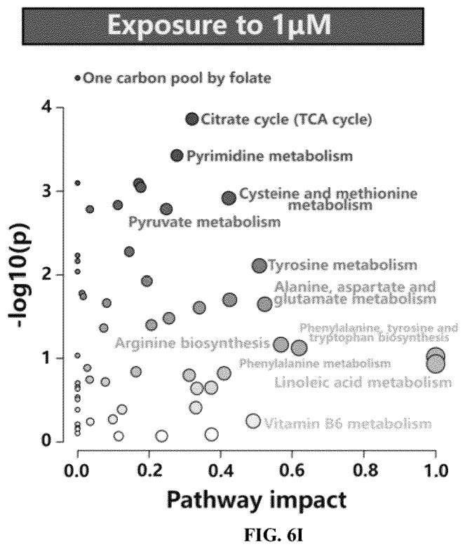

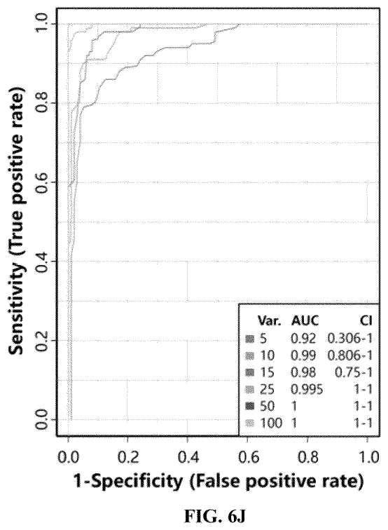

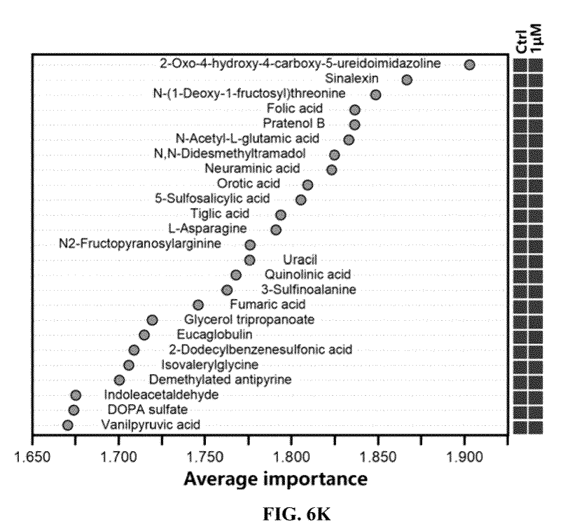

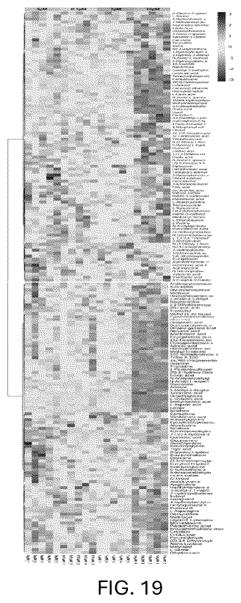

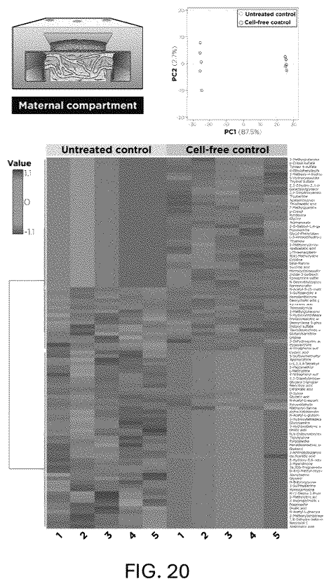

Inspired by this knowledge gap, we conducted unbiased, global analysis of the metabolome of our exposure model, towards the goal of discovering specific markers and molecular signatures indicative of cadmium-induced metabolic changes in the fetal and maternal compartments. In this study, we treated our model established using primary cells with three select concentrations of cadmium chloride (0.1, 1, and 10 μM). For analysis, we used device effluent collected from the maternal and fetal chambers separately over the course of 24-hour exposure (FIG. 6A). Dimensionality reduction using principal component analysis (PCA) of our data acquired from the maternal compartment indicated distinct metabolic profiles at different exposure levels, as illustrated by a separation between dose-dependent clusters (FIG. 6B). The heatmap representation of 183 most differentially regulated metabolites identified by hierarchical cluster analysis demonstrated that i) the overall patterns of metabolite expression were similar between the untreated control and 0.1 μM groups and that ii) exposure to higher doses (1 and 10 μM) resulted in noticeable changes in their metabolic profiles, especially at 10 μM, in comparison to control and the 0.1 μM group (FIG. 6C, FIGS. 19, 20).

Quantitative comparison of specific metabolites between these groups provided further insight into how cadmium might alter placental metabolism in our model (FIG. 6D, FIG. 21). For example, our data indicated elevated pyrimidine metabolism as a result of cadmium exposure at 1 and 10 μM, which was evidenced by substantially increased levels of its metabolic product, uridine (FIG. 6D). This is a notable finding for verifying the physiological relevance of our exposure model as in vivo data from mouse studies have identified significantly altered pyrimidine metabolism as one of the key metabolic signatures associated with spontaneous abortion due to prenatal exposure to benzophenone-3. Cadmium also increased asparagine and serine metabolism in our model (FIG. 6D). Although these pathways have not been investigated in the context of chemical toxicities in the placenta, elevated levels of asparagine and serine were previously shown by metabolomic analysis of the human placenta with fetal growth restriction. Glucosamine was another upregulated marker at higher doses of cadmium known to play a critical role in the synthesis of glycosylated proteins and lipids (FIG. 6D). No evidence exists implicating glucosamine in human placental toxicity of cadmium, but it has been associated with altered fetal growth in mothers with diabetes.

Exposure to the higher doses also led to the reduced activity of several metabolic pathways. This was illustrated by the decreased production of metabolites, such as cis-aconitic acid that plays an essential role in energy production and removal of toxins and folic acid that is an important contributor to placental development and fetal growth (FIG. 6E). In response to lower-level exposure at 0.1 μM, metabolism in the maternal compartment remained largely unaffected, but a small number of metabolites still showed significant changes compared to control, including tiglic acid and D-xylose (FIGS. 6D, FIG. 11). In addition to these examples, our analysis identified many other upregulated or downregulated metabolites with previously unknown links to human placental toxicities of cadmium and other toxic environmental metals (Table 1).

We then performed pathway impact analysis to gain further insight into the metabolic consequences of cadmium exposure in our model. Comparison of the 10 μM exposure condition with control allowed us to identify and rank 57 metabolic pathways that were most significantly altered by cadmium (FIG. 6F). These included pyrimidine and folate metabolism described above, as well as purine metabolism involved in fulfilling the demanding need for energy production in the placenta, nicotinate and nicotinamide metabolism essential for redox reactions that has been shown to change in gestational diabetes, and metabolism of amino acids such as alanine and glutamine that play key roles in supporting fetal growth and metabolism.

Furthermore, we carried out multivariate receiver operating characteristics (ROC) analysis to identify potential metabolite biomarker candidates that may be associated with cadmium toxicity in the human placenta (FIG. 6G). This analysis using the 25-feature model showed i) reduction in orotic acid and lubimonol and ii) increased levels of L-aspartic acid, medicagol, and 5-methyl-2-thiophenecarboxaldehyde as the most predictive signatures of metabolic changes due to placental exposure to 10 μM cadmium in our model (FIG. 6H).

Finally, the same set of analysis was performed for the intermediate-level (1 μM) exposure condition (FIGS. 6I-6K). The metabolic pathways perturbed by this condition included some of those discovered at 10 μM (e.g., pyrimidine and folate metabolism) but data also revealed new ones, including taurine and hypotaurine metabolism, which has been shown by rodent studies to protect trophoblasts from oxidative stress-induced cytotoxicity and sphingolipid metabolism whose alterations have been associated with gestational diabetes (FIG. 6I). Of note, the top 25 biomarker candidates predicted by ROC analysis for this condition and those for the high-dose condition (10 μM) only shared five metabolites (FIG. 6K), suggesting the possibility of developing distinct metabolic signatures that may distinguish placental responses to these two exposure conditions.

### Metabolomic Profiling of the Cadmium Exposure Model—Fetal Compartment

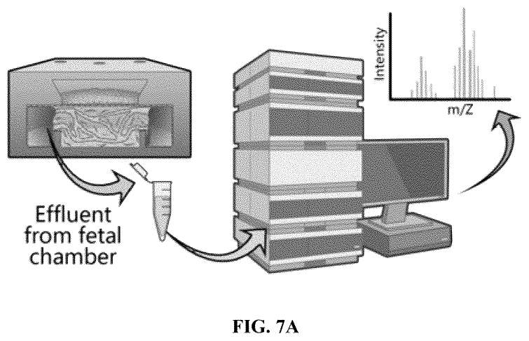

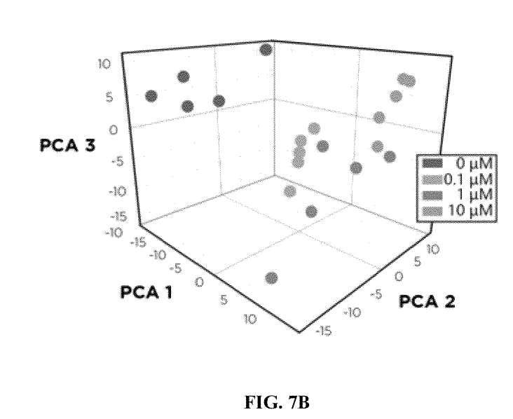

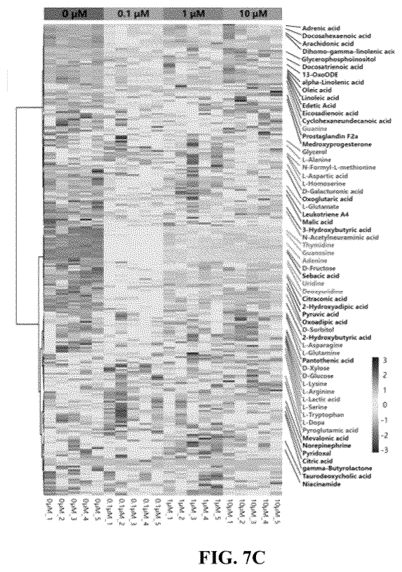

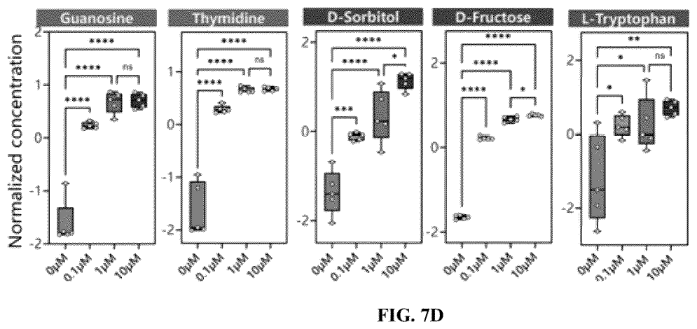

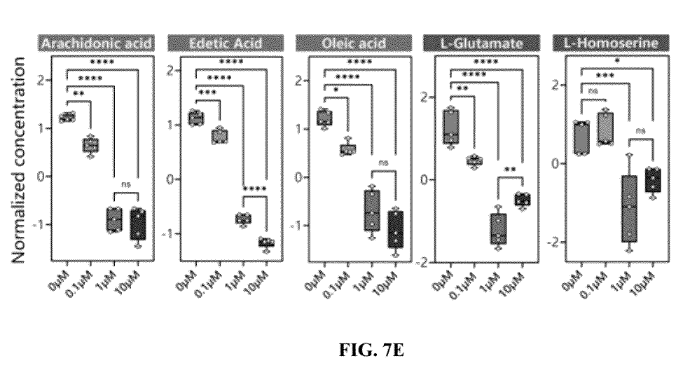

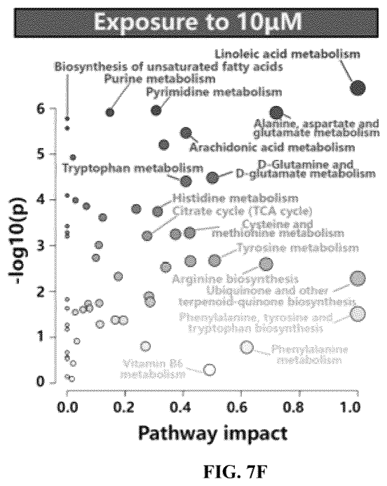

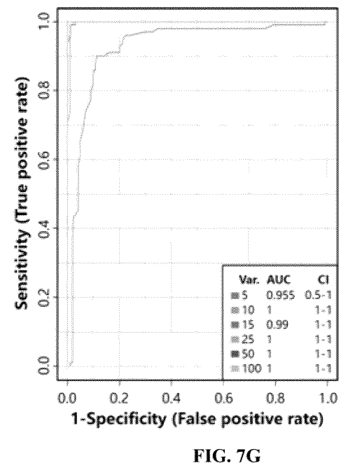

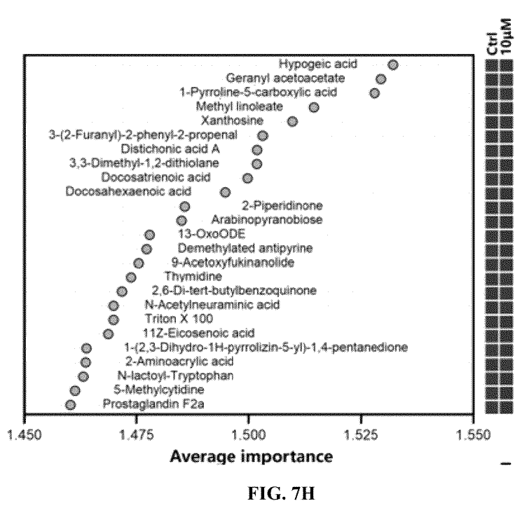

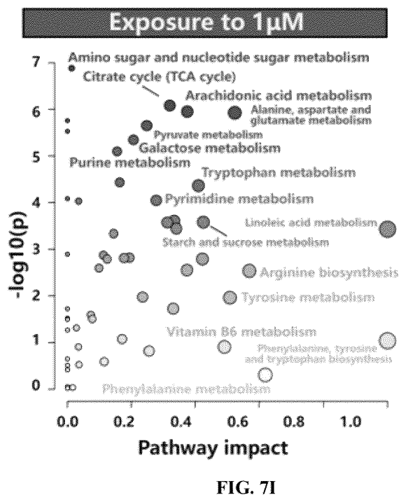

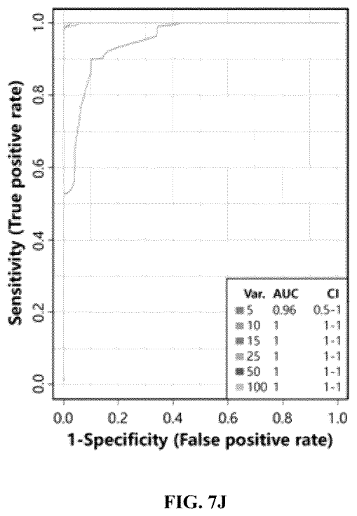

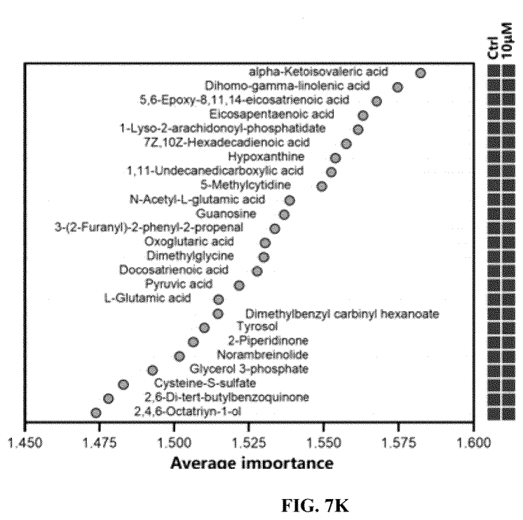

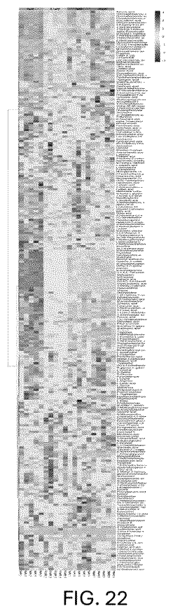

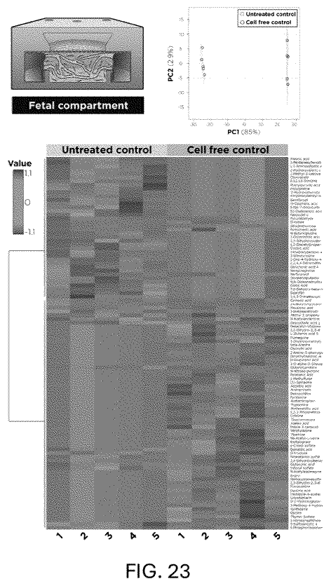

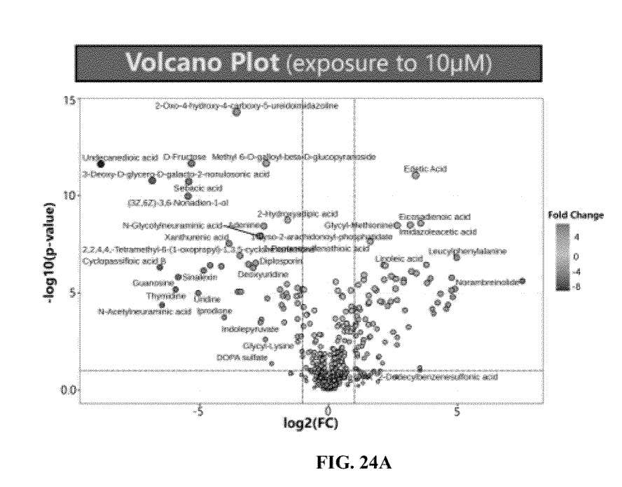

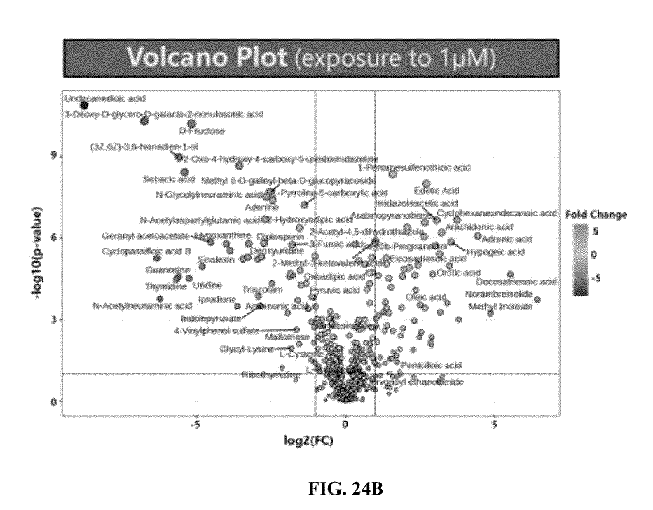

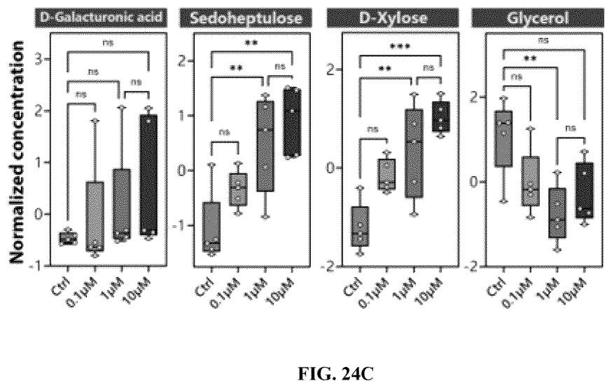

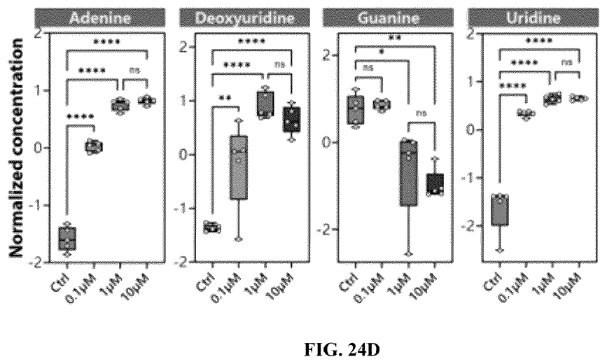

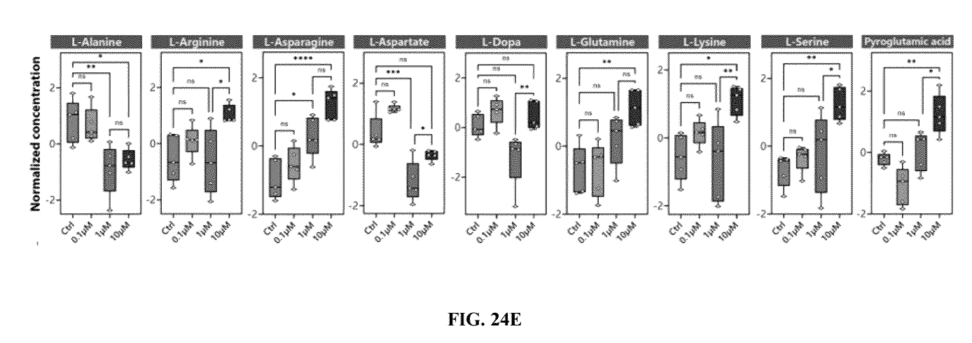

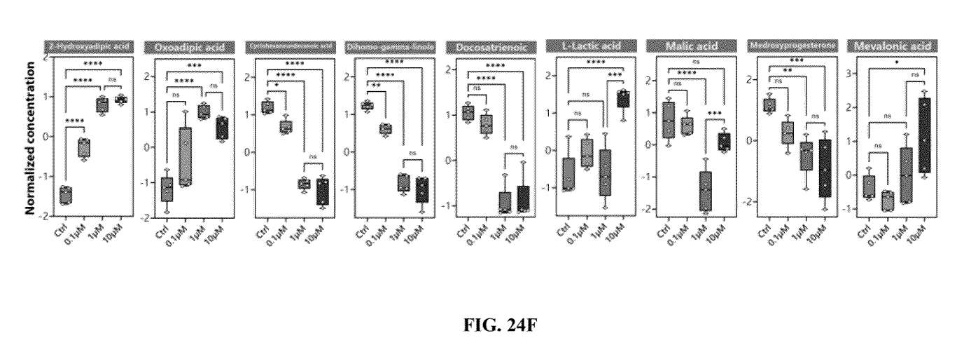

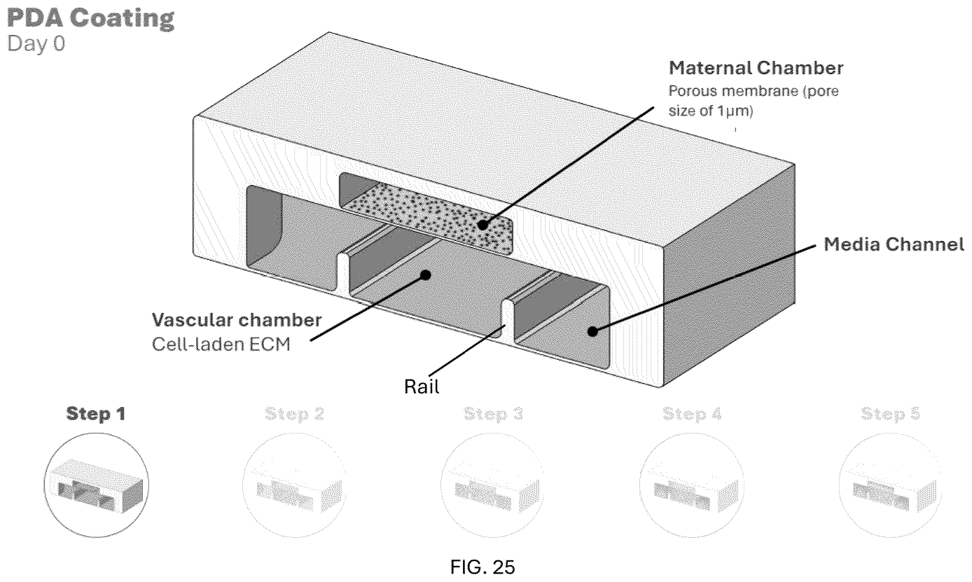

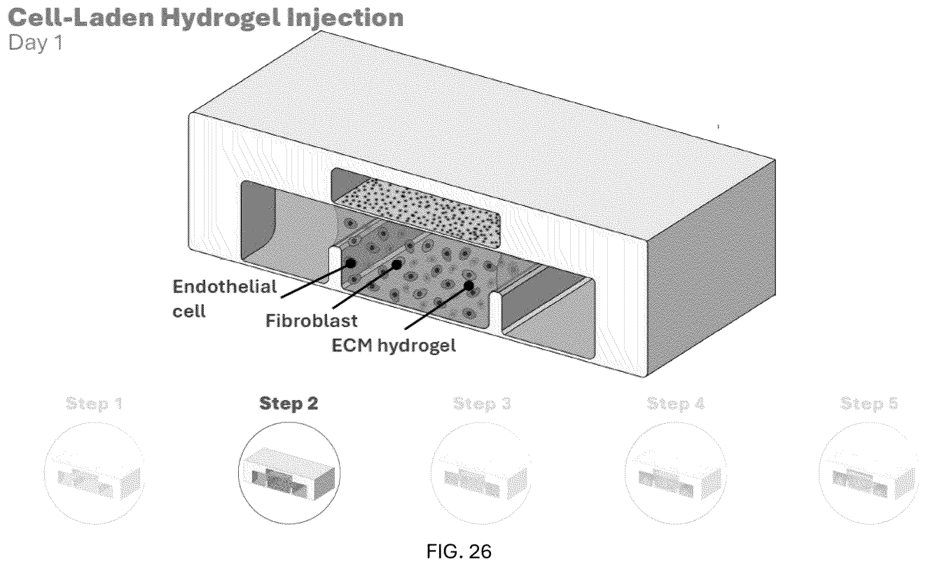

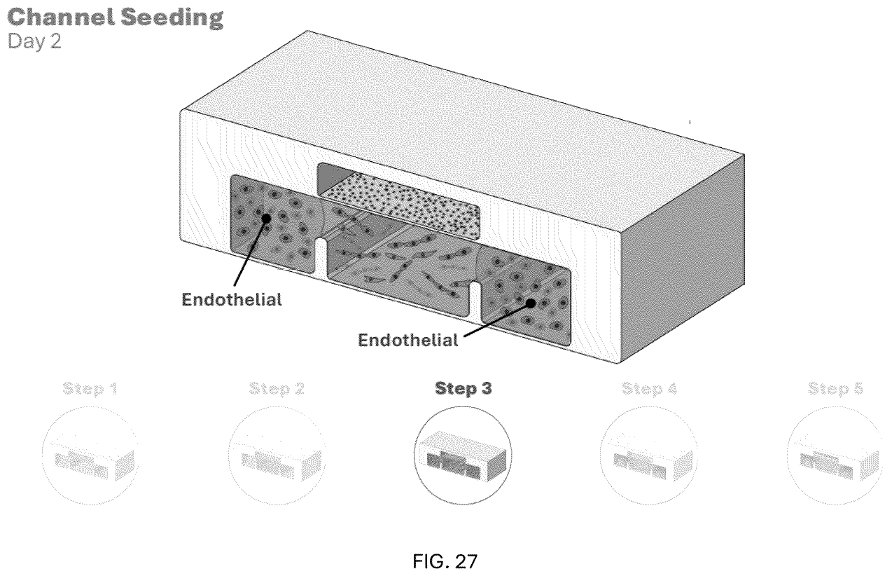

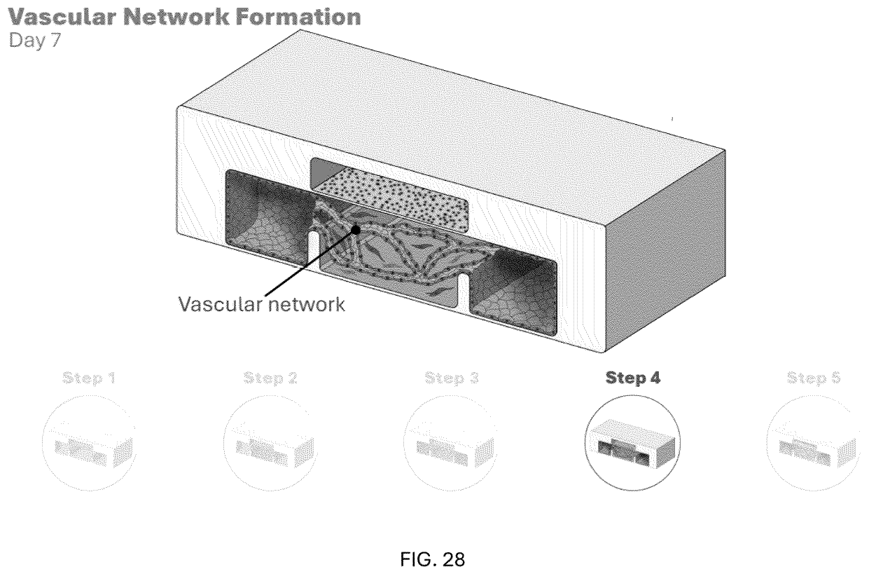

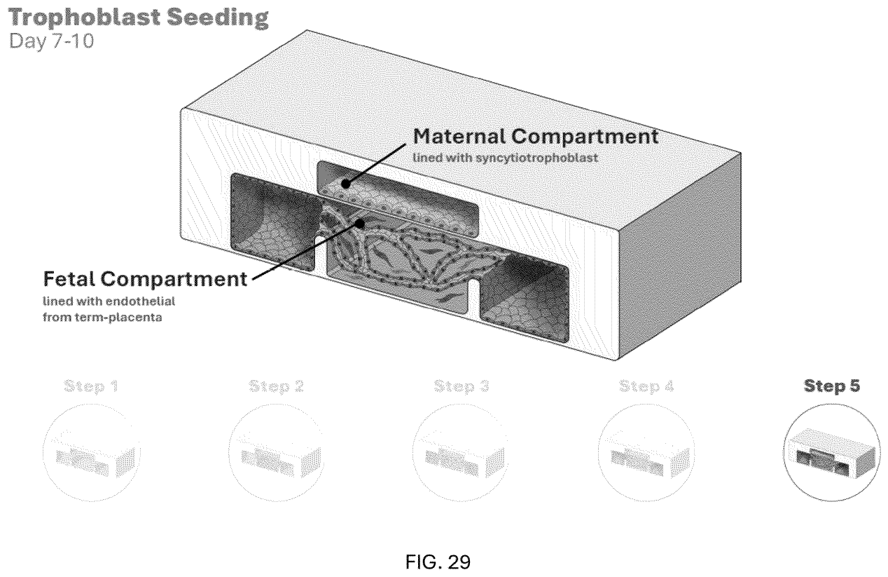

Analysis of metabolites present in the perfusate of the fetal compartment demonstrated similar dose-dependent profiles, but in comparison to what was measured in the maternal chamber, many metabolites began to show noticeable changes even in the lower-level (0.1 μM) exposure condition (FIGS. 7A-7C, FIGS. 22, 23). Guanosine was one of them that was significantly upregulated at 0.1 μM, and its level increased further when the concentration was raised to 1 μM (FIG. 7D). Cadmium exposure also resulted in substantially increased production of sorbitol at 0.1 μM and higher (FIG. 7D). Although these metabolites have not been described in the context of environmental metal toxicity in the placenta, clinical studies have shown their altered production as an indicator of abnormal fetal development and pregnancy complications. Increased levels of guanosine, for example, were among the key findings of metabolomic analysis of human placentas with spontaneous preterm birth⁸⁰. Similarly, sorbitol accumulation in fetal tissues has been suggested to contribute to the development of fetal defects in diabetic pregnancy. In addition to these markers, our analysis showed increased production of many other metabolites without known associations with pregnancy (FIG. 7D, FIG. 24, Table 2).

We also found that cadmium exposure led to a decrease in the levels of several metabolites on the fetal chamber side. Examples included: arachidonic acid, an essential fatty acid critical for proper neural development in the fetus, oleic acid, which plays an important role in maintaining early-stage pregnancy, glutamate, a major substrate for fetal energy metabolism, and many others that have not been studied in relation to placentation and pregnancy (FIG. 7E. FIG. 24, Table 2). Interestingly, a large fraction of the significant metabolic changes in the fetal compartment were seen in fatty acids, indicating the capacity of cadmium to disrupt lipid metabolism of fetal tissues in our model. Consistent with this finding, pathways involved in the metabolism of fatty acids (e.g., arachidonic acid, linoleic acid) were shown by our analysis to be among the most significantly affected pathways in both the 1-μM and 10-μM groups (FIGS. 7F, 7i). Notably, these changes were not observed in the maternal compartment, suggesting that disrupted fatty acid metabolism may represent a distinctive feature of the fetal response to cadmium exposure.

Comparative analysis of the highly ranked pathways in the maternal and fetal compartments also revealed several shared features of altered metabolism. For example, cadmium exposure significantly affected pathways involved in the metabolism of nucleosides, including elevated levels of uridine and guanosine. Additionally, both compartments showed altered activities of pathways regulating organic acid metabolism, such as those involving oxoglutarate and citric acid, known to play a key role in central carbon metabolism and redox homeostasis.

Our biomarker analysis using the ROC curve yielded a set of differentially regulated metabolites associated with fetal-specific effects of cadmium. In case of higher-level exposure at 10 μM, metabolites with the highest predictive values included fatty acids and fatty acid esters, such as hypogeic acid, methyl linoleate, docosatrienoic acid, and docosahexaenoic acid, all of which were present in decreased amounts as a result of cadmium exposure (FIG. 7H). The fetal compartment-specific biomarker candidates also included geranyl acetoacetate, 1-pyrroline-5-caboxylic acid, and xanthosine whose levels were elevated by cadmium (FIG. 7H). Data from the 1-μM group revealed a different set of fatty acids and other types of metabolites as predictive biomarkers (FIGS. 7J, 7K). Specifically, fetal metabolic changes due to cadmium exposure at this level were represented by decreased levels of a-ketoisovaleric acid, dihomo-γ-linolenic acid, 5,6-epoxy-8,11,14-eicosatrienoic acid, and eicosapentaenoic acid, as well as increased production of 1-lyso-2-arachidonoyl-phosphatidate and hypoxanthine, to name a few with the highest predictive values (FIG. 7K). To our knowledge, most of these metabolites and their pathways have not been investigated in the context of placental exposure to environmental metals.

### Ex Vivo Validation of Placental Responses to Cadmium

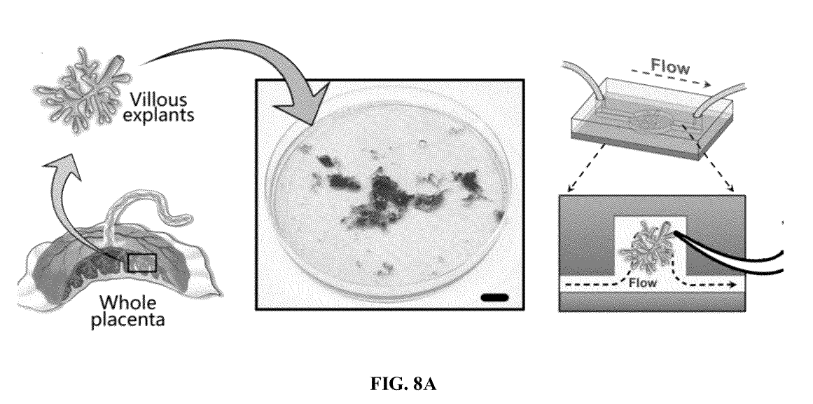

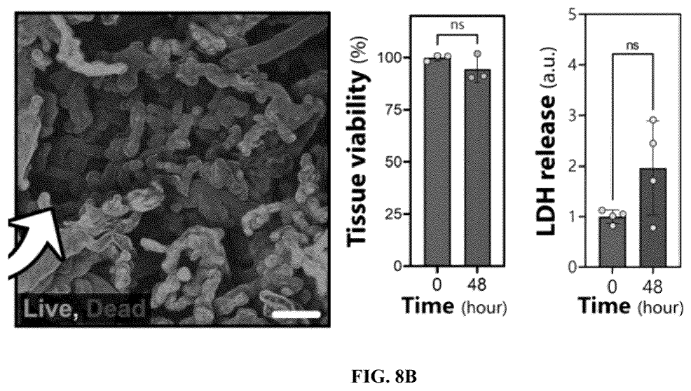

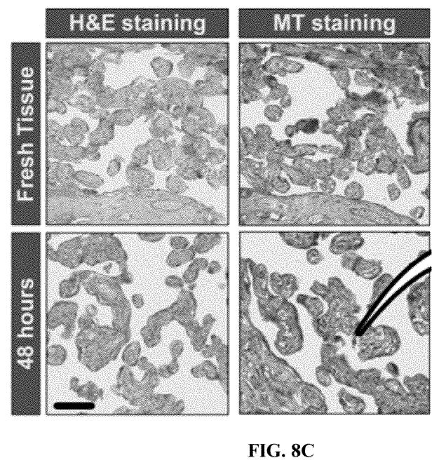

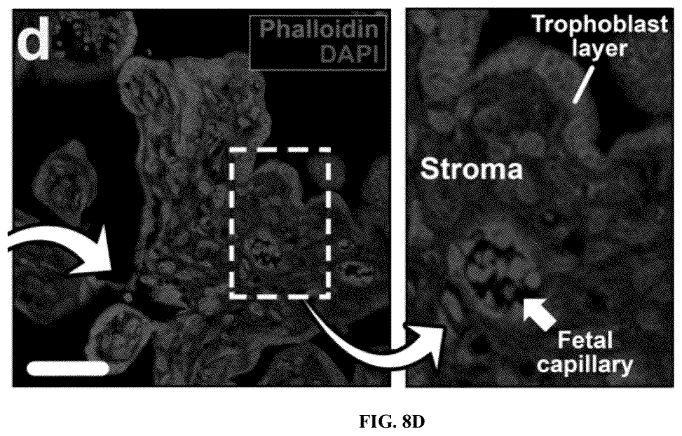

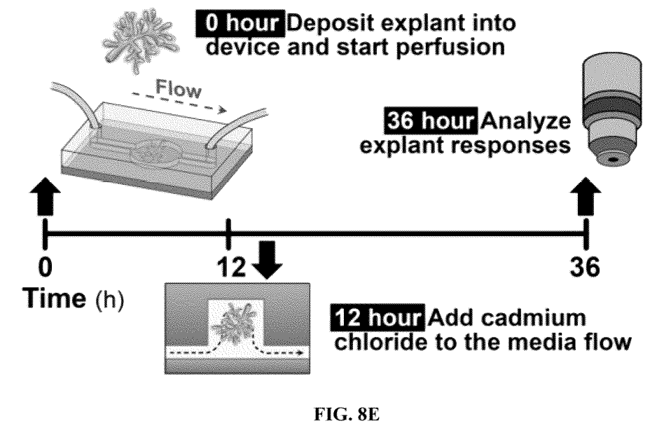

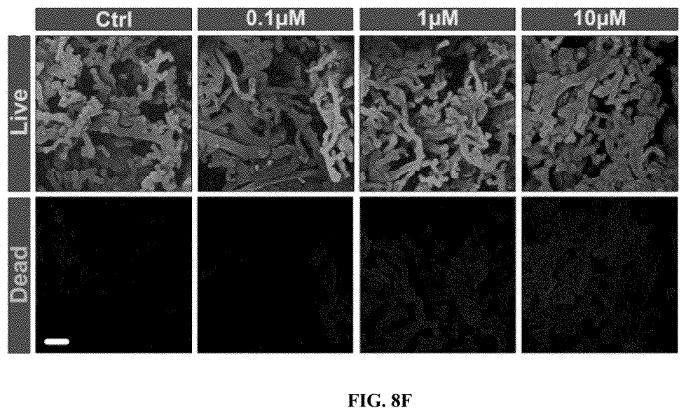

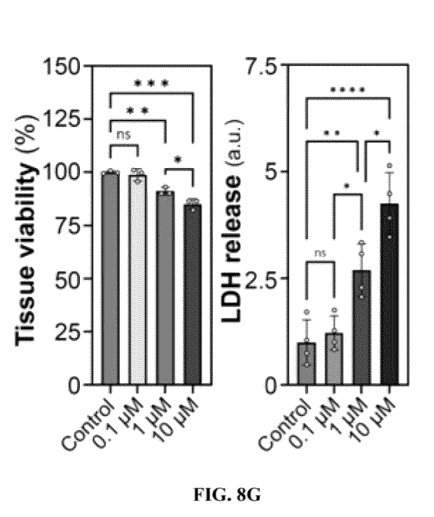

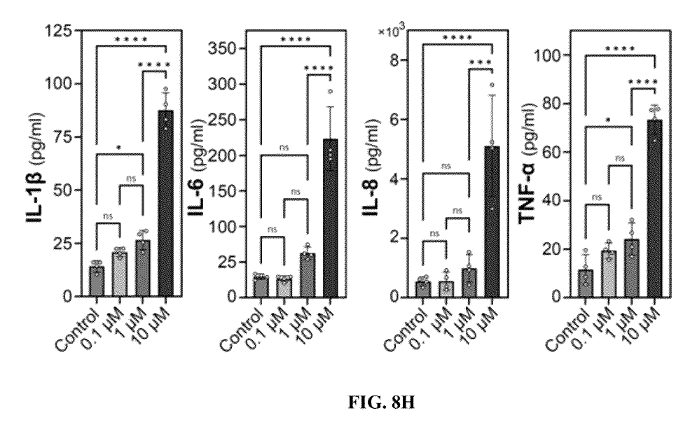

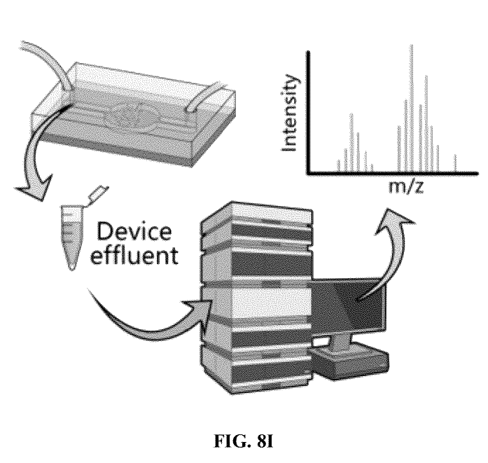

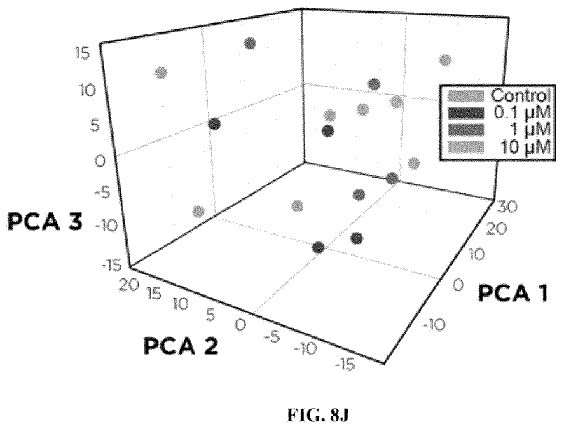

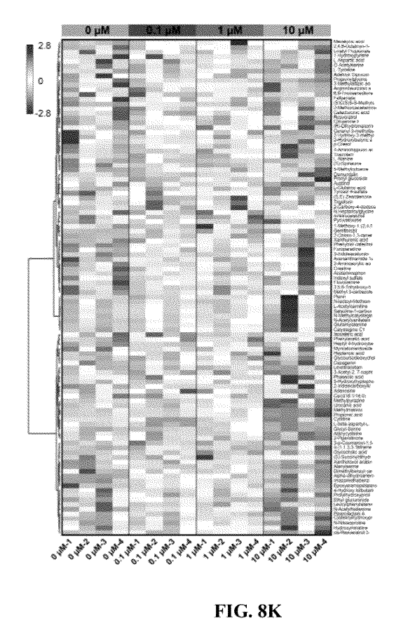

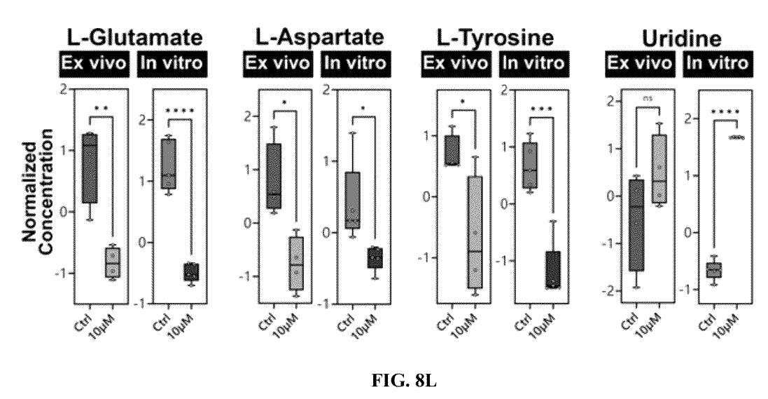

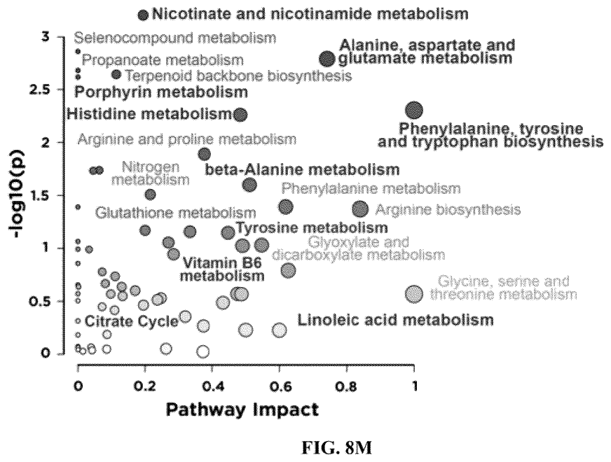

Various adverse responses to cadmium exposure in our bioengineered model led us to ask whether we could validate some of the key findings of this in vitro study. To this end, we used living villous explants isolated from term human placentas to construct an ex vivo model in which the tissue explants were housed in a 4-mm circular chamber created in a 3D-printed PDMS device and perfused continuously with explant culture media to simulate hemodynamic flow in the intervillous space (FIG. 8A). This culture condition was effective for preserving tissue viability (FIG. 8B) and the villous structures of the explants (FIG. 8C). Notably, the trophoblast layer and its underlying stroma containing fetal capillaries were clearly visible in the histological examination of the explants cultured for 48 hours with perfusion (FIG. 8D), demonstrating the intact microarchitecture of the placental barrier in the ex vivo model.

Modeling placental exposure to cadmium in this system was achieved by i) first depositing and culturing villous explants in the device for 12 hours and ii) then adding cadmium chloride at defined concentrations (0.1, 1, or 10 μM) to the media flowing through the culture chamber (FIG. 8E). After 24-hour treatment, the chorionic villi in the explants appeared to retain their original morphology and branching structures, regardless of cadmium concentration (FIG. 8F). At higher doses, however, cadmium had negative effects on cell viability, as illustrated by a significant increase in the fraction of membrane-compromised cells and the level of LDH in device effluent at 1 and 10 μM (FIG. 8G). In particular, 24-hour treatment of the explants with 10 μM cadmium chloride led to a 4.5-fold increase in LDH release from the untreated control, which was in good agreement with the corresponding increases observed in the maternal (2.24-fold) and fetal (1.51-fold) compartments of our in vitro model under the same exposure conditions (FIGS. 4D, 4G).

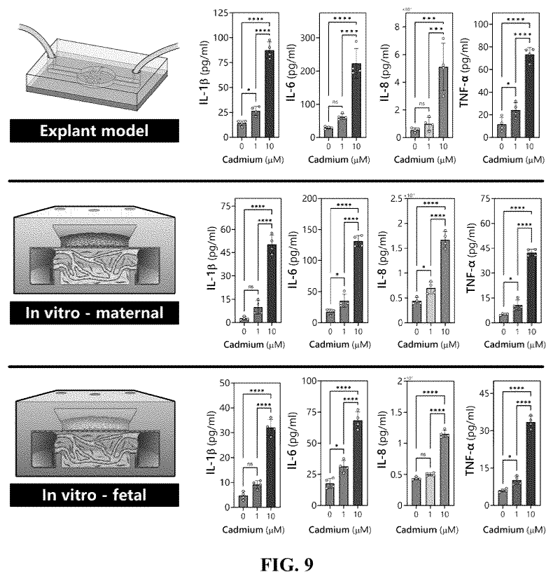

Analysis of pro-inflammatory cytokines in the effluent samples showed statistically insignificant increases in the levels of IL-1β, IL-6, IL-8, and TNF-α at 0.1 and 1 μM as compared to control (FIG. 8H). This result was consistent with the measurement of the same cytokines in our microphysiological system (FIGS. 4A, 4B, FIG. 9), with the exception of the significantly elevated production of IL-6 in the fetal compartment of the in vitro model treated with 1 μM cadmium. When the concentration was raised to 10 μM, all of the cytokines measured increased their levels by a factor of at least 3, indicating highly inflammatory phenotype of the ex vivo model caused by the high cadmium concentration (FIG. 8H). The mean fold increase for all cytokines in the explants was 7.3, which was comparable to that evaluated in our in vitro system (9.55 and 4.72 for the maternal and fetal compartments, respectively). (FIGS. 4A, 4B, FIG. 9).

Finally, extending the scope of this analysis, we performed in vitro-ex vivo comparison of data generated by global metabolomics analysis (FIG. 8A). Both principal component analysis and heatmap visualization showed dose-dependent clustering of metabolomics data from our explant model (FIGS. 8J, 8K). The metabolic profiles, as illustrated by the heatmap, revealed similarities between untreated control and the 0.1 μM group, but they appeared noticeably different when the cadmium concentration was higher (FIG. 8K). Specifically, the overall patterns of differential regulation of the top-ranked metabolites were reversed as a result of cadmium exposure at 1 μM, and this change became even more pronounced at 10 μM (FIG. 8K). Although qualitative, this dose-dependent trend was similar to cadmium-induced metabolic changes observed in the maternal (FIG. 6C) and fetal (FIG. 7C) compartments of our microengineered model.

Since effluent samples used in this analysis contained a mixture of metabolites derived from both the trophoblast and fetal vascular sides of the villous explants, in vitro-ex vivo comparison of individual metabolites in a compartment-specific manner was not feasible. Nevertheless, some top-ranked metabolites commonly detected in both models exhibited highly similar responses to cadmium exposure (FIG. 8L). For example, L-glutamate and L-aspartate, two core intermediates in the alanine, aspartate, and glutamate metabolism pathway, were significantly downregulated at 10 μM in both the explant model and the maternal compartment of our in vitro system.

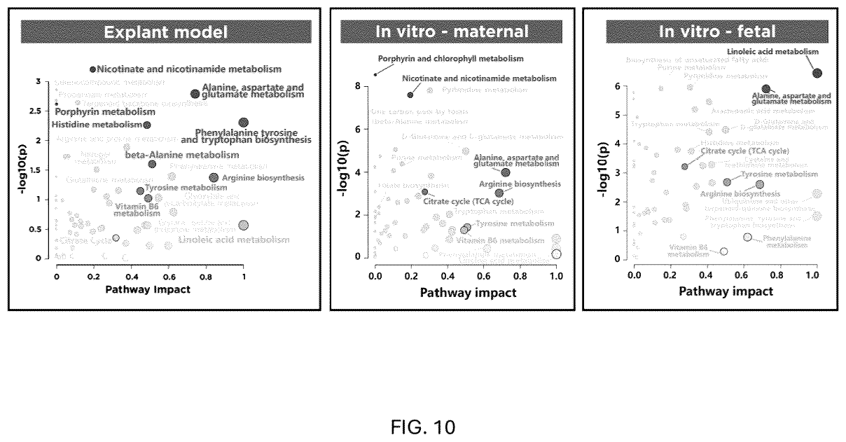

Importantly, pathway impact analysis revealed substantial overlap between the metabolic changes observed in the ex vivo and in vitro models following cadmium exposure (FIG. 8M, Extended Data FIG. 10). In the 10 μM treatment group, top-ranked pathways identified in the explant model included nicotinate and nicotinamide metabolism and alanine, aspartate, and glutamate metabolism (FIG. 8M, Extended Data FIG. 102), both of which were also among the most significantly altered pathways in the maternal compartment of our microphysiological model exposed to the same cadmium concentration (FIG. 6F). Similarly, phenylalanine, tyrosine, and tryptophan biosynthesis and linoleic acid metabolism, two of the most highly impacted pathways in the ex vivo model (FIG. 8M, Extended Data FIG. 10), were also detected in the fetal compartment of the in vitro model (FIG. 7F). Quantitatively, approximately 48% and 36% of the top 25 pathways in the explant model overlapped with those in the maternal and fetal compartments of our microengineered in vitro system, respectively.

While more rigorous and quantitative analysis is needed for full model validation, these results demonstrate the ability of our bioengineered exposure model to produce in vitro data that correlate with adverse biological responses that can be measured in living human placental explants.

### Discussion

In this disclosure, we have demonstrated how the key features of the maternal-fetal interface in the human placenta can be recapitulated using microengineered cell culture systems and how these platforms can be leveraged to model and investigate adverse biological responses of the placental barrier to environmental toxicants.

A critical aspect of model construction in this work was to use primary human trophoblasts isolated from the chorionic villi of the human placenta and cultured in physiologically relevant conditions maintained at 5% oxygen. This represents a notable improvement over existing in vitro models of the human placental barrier, which typically use human trophoblast cell lines, such as BeWo and JEG, cultured at 20% oxygen. The physiological oxygen environment in our system enabled primary trophoblasts to maintain their proliferative capacity and more faithfully recapitulate the properties of native tissue, as illustrated by spontaneous syncytialization and increased hCG production.

When compared to a conventional Transwell-based in vitro model, the microengineered placental barrier was found to be more resistant to cytotoxic effects of cadmium administered at a range of 5 to 100 μM, despite the fact that both systems were created using the same batches of primary trophoblasts cultured at 5% oxygen (FIGS. 4B-4D). This may be attributed to the effect of fluid flow in the microphysiological system, which was not present in static Transwell cultures. Our data show that flow conditions in our model improve barrier integrity and upregulate trophoblast expression of key maturation markers and efflux transporters that can play a protective role against cadmium toxicity (FIGS. 3O-3Q). The difference in the structural organization of tissue constructs may be another contributing factor. The Transwell model consisted of two opposing cell layers separated by a thin membrane, whereas our microengineered system contained a hydrogel-filled stromal compartment beneath the membrane, which likely provides additional physical barrier to cadmium transfer across the maternal-fetal interface. This may explain substantially reduced cadmium-induced tissue damage in the fetal vasculature of the microengineered model.

To contextualize these findings in relation to human situations, it is important to note that the levels of cadmium in typical scenarios of human environmental exposure rarely exceed 10 μM and that the human placenta is usually not associated with severe tissue injury and inflammation under such conditions. The prevailing hypothesis is that that cadmium may exert more subtle toxic effects by disrupting biological signaling essential for regulating placental barrier phenotype and function, rather than causing direct cytotoxic damage. Consistent with this idea, exposure of our model to 0.5, 1, and 5 μM of cadmium did not compromise cell viability, yet it still elicited adverse cellular and tissue responses, including reduced hormone production, decreased maternal-to-fetal glucose transfer, increased cytokine production, and altered transporter expression. The observation that the Transwell model exhibited signs of tissue injury at these lower levels of cadmium exposure (FIGS. 4B-4G) suggests that our engineered model may have the capacity to more accurately represent physiological human placental responses to cadmium and capture nuanced functional impairments that may be missed in conventional in vitro systems.

Our data provided additional evidence of cadmium toxicity by demonstrating disrupted metabolic activities in the exposure model. Research has shown adverse effects of cadmium and other toxic metals on metabolic function of various organs, but their impact on placental metabolism has not been reported previously. Notably, our analysis revealed a high degree of similarities between some of the specific metabolic changes observed in our model and those identified in metabolomic studies of human placentas associated with pregnancy complications, including spontaneous preterm birth, gestational diabetes, spontaneous abortion, and fetal growth restriction. Although our system does not directly model the effects of cadmium on fetal development, these findings suggest disrupted placental metabolism as a potential mechanism contributing to cadmium-associated pregnancy complications, which remains a major knowledge gap in the field.

Among the key findings of this study was the cadmium-induced disruption of lipid metabolism in the fetal compartment. The placenta is known to take up lipids from the maternal circulation and convert them into free fatty acids to support its own growth, while also transporting them to the fetal circulation to meet the high metabolic needs of the growing fetus. This highly regulated process becomes even more important in late stages of gestation as lipids are essential for fetal neurodevelopment. Studies have shown that placental processing of lipids can be impaired by maternal conditions like obesity, contributing to placental maladaptation and pregnancy complications common in such conditions. It remains unknown, however, whether and how placental lipid metabolism is affected by prenatal exposure to toxic metals. Our data suggest that cadmium may have the ability to interfere with lipid metabolism in the human placenta to change the levels of fatty acids in the fetal circulation, which may have negative implications for fetal development.

Also worth noting is the protective role of BCRP demonstrated in the exposure model. Although the function of these transmembrane proteins are well studied for cellular export of ions, macromolecules, toxins, and drugs in various organs, their activities during prenatal cadmium exposure remain poorly understood. BCRP, one of the two most abundant ABC transporters in the human placenta, was recently shown to protect cultured trophoblasts from cadmium. Supporting this finding in a more complete in vitro model, our data show the capacity of BCRP to reduce cadmium-induced tissue damage and inflammation in both the maternal and fetal compartments during human exposure conditions. We also found that BCRP provides greater protection than other ABC transporters in our system, such as MDR1 and MRP2. These findings reinforce the notion of ABC transporters as innate defense mechanisms against foreign substances and highlight the specific role of BCRP-mediated efflux transport function in placental cadmium toxicity. We identified dose-specific metabolite biomarkers that indicate cadmium-induced metabolic changes in the maternal and fetal compartments of our model.

### Methods

### Fabrication of Cell Culture Devices

Devices used for culturing human placental cells in this study were fabricated using soft lithography. Briefly, poly (dimethyl siloxane) (PDMS, Sylgard 184, Dow Corning) monomer base was mixed with a curing agent (10:1, w/w) and poured onto 3D-printed molds manufactured by stereolithography (Protolabs). After degassing in a desiccation chamber (Bel-Art Inc.) for 1 hour, PDMS was cured in an oven at 65° C. for 2 hours. Subsequently, a Whatman Cyclopore Polycarbonate Thin Clear membrane with 1 μm pores (Cytiva, USA) was punched using a 7 mm biopsy punch (Acuderm Inc., USA) and sealed against the middle PDMS layer using uncured PDMS as a glue. Next, fluidic access ports were generated in the topmost PDMS layer (tubing layer) using a 1 mm biopsy punch (Integra, Inc.), and this layer was bonded to the upper PDMS layer containing the maternal chamber. The device assembly was then placed in an oven for 1 hour and stored in a container until use.

### Cell Culture

Human primary cells used in this study were obtained from the Amnion Foundation (NC, USA) that included human cytotrophoblast cells (Cat #1230), placental stromal fibroblasts (Cat #1250), MVECPRO2, germinal-origin microvascular endothelial cells (Cat #1245), and Hofbauer cells (Cat #1220). Human placental microvascular endothelial cells and placental fibroblasts were seeded and grown in Corning TC-treated T-75 tissue culture flasks. The endothelial cells and fibroblasts were cultured in microvascular endothelial cell growth medium (MVECPRO2: Lonza EGM-2 MV) and fibroblast media (Stromal cells/fibroblasts; Lonza FGM-2), respectively. These cells were used for creating engineered placental tissues after one passage. Human cytotrophoblast cells were cultured using cytotrophoblast culture media (CTBPRO2GRO: Amnion Foundation) in an incubator maintained at 5% oxygen.

### Tissue Production in the Fetal Chamber

After device fabrication, the PDMS chambers were filled with 70% ethanol and incubated for 1 minute, after which ethanol was removed by applying vacuum aspiration to access ports. Subsequently, the device was sterilized for 20 minutes using ultraviolet (UV) light. To generate vascularized stromal tissues in the fetal compartment, a 100-μl mixture of 10 mg/ml fibrinogen (Millipore Sigma Cat. F8630-5G) in DPBS, thrombin (10 U/ml), microvascular endothelial cells (5× 10⁶ cells/ml), and placental fibroblasts (5× 10⁶ cells/ml) were injected into the fetal chamber of the lower device layer compartment of a microfabricated device. For devices containing Hofbauer cells, primary human Hofbauer cells (Amnion Foundation) were first labeled with CellTracker™ red CMFDA and added to the ECM-cell mixture at a final concentration of 1×10⁶ cells/ml, which was then injected into the fetal chamber of our device. The seeded device was placed in an incubator at 37° C. for 15 minutes to induce gelation of the mixture. Subsequently, the side channels were seeded with endothelial cells (5× 10⁶ cells/ml). After cell attachment, the fetal chamber of the device was connected to a computer-controlled flow pump driven at a volumetric flow rate of 100 μl/h.

### Tissue Production in the Maternal Chamber

To improve cell adhesion, the polycarbonate membrane surface was coated with fibronectin/gelatin (0.1 mg/mL) for 2 h at 37° C. and rinsed with PBS before seeding. Primary cytotrophoblasts were seeded onto the upper membrane surface at a seeding density of 2.5×10⁶ cells/ml, incubated for 1.5 h, and cultured under continuous perfusion (100 μl/h). BeWo wild-type and BCRP knockdown cells were cultured in DMEM: F12 with 10% FBS and 1% penicillin-streptomycin. After membrane coating, BeWo cells (2.5× 10⁶ cells/mL) were seeded into the maternal chamber, incubated for 1.5 h, and perfused at 100 μl/h. when BeWo cells were used in our model, syncytialization was induced by treating the cells with 50-100 μM forskolin.

### Transwell Culture

Transwell culture of primary human placental cells was established for comparative analysis of biological responses to cadmium. This model was created in a Transwell insert (Corning, NY, USA catalog #38024) containing a semipermeable membrane with a pore size of 0.4 μm. First, microvascular placental endothelial cells were plated on the lower side of the membrane insert at a concentration of 5×10⁶ cells/ml while the insert was kept inverted. After 1.5 hours of incubation at 37° C., 5% CO₂ to permit cell attachment, the endothelial cell-seeded insert was placed in a culture well containing fresh EGM-2 MV media. Following this step, trophoblast cells were seeded onto the upper side of the membrane at 5×10⁶ cells/ml. During culture, media were changed every 24 hours until 100% confluency was reached.

### Cadmium Exposure

The cadmium used in this study was sourced from CdCl2 (Sigma-Aldrich, catalog #202908), prepared as a stock solution in distilled deionized water, and appropriately diluted with medium before application. To generate an exposure model, the maternal chamber was perfused with trophoblast media containing defined concentrations of cadmium (0, 0.1, 0.5, 1, 5, 10, and 50 μM). During exposure, the devices were maintained at 37° C. and 5% CO₂.

### Construction of Explant Model

Human placental tissues were obtained from the Amnion Foundation (Durham, NC) as de-identified research samples under vendor-provided protocols. The use of these tissues was determined to be IRB-exempt by the University of Pennsylvania. The cryoprotected tissue in 5 ml vials were rapidly thawed in a 37° C. water bath prior to use. Thawed tissue was transferred directly into 40 ml of cold complete growth medium containing 10% fetal bovine serum and gently rocked at 4° C. for 15-20 minutes. Samples were then allowed to settle by gravity or gently centrifuged at 200×g for 5-10 minutes. The spent medium was carefully removed prior to further processing or culture. Small biopsies (4 mm) of villous explants were dissected and placed into our devices. Explants were cultured in the device under continuous flow (100 μl/h) of phenol red-free high glucose DMEM/F12 medium supplemented with 10% FBS and 1% penicillin/streptomycin for 12 hours to stabilize before cadmium exposure.

### Histology and Immunofluorescence Staining and Microscopy

For immunofluorescence staining, tissues were first fixed in a 4% PFA solution at 4° C. for 4 hours and then permeabilized in 1% Tween-20 and 10% BSA at 4° C. for 2 hours. Primary antibodies were applied at manufacturer-recommended dilutions or at 1:100 dilution, whichever was more concentrated, and incubated at 4° C. overnight. On the next day, tissues were washed with DPBS containing 0.1% Tween-20 and 10% BSA and left overnight. Secondary antibodies, if required, were incubated overnight, followed by another day of washing in the same solution. DAPI and Phalloidin were added at dilutions of 1:5000 and 1:1000, respectively. Following staining, imaging was conducted using confocal microscopy (Zeiss LSM 800) with 10×0.45 NA and 63×1.4 NA objectives.

For histological analysis, paraffin-embedded tissue sections were deparaffinized and rehydrated by sequential immersion in xylene (3×), 100% ethanol (2×), followed by graded ethanol solutions (95%, 90%, 80%, and 70%) and finally distilled water. Sectioning and staining were performed by the Molecular Pathology and Imaging Core (MPIC) and the Penn Center for Musculoskeletal Disorders (PCMD) Histology Core.

Antibodies used for immunofluorescence analysis included CD31 (Alexa Flour 488 Anti-CD31, Abcam ab215911), E-Cadherin (Abcam ab40772 and ab1416), GLUT-1 (Abcam ab115730), CD163 (Abcam ab182422, Invitrogen MA5-17716), ZO-1 (Invitrogen 33-9100, Invitrogen 61-7300), ICAM-1 (Invitrogen 1A29), Fibronectin (Invitrogen 53-9869-82), α-SMA (Invitrogen 14-9760-82, R&D Systems MAB1420), and BCRP (Santa Cruz sc-18841). The complete list of antibodies is shown in Table 3.

### Measurement of Cell Viability, LDH Release, and Apoptosis

The viability of trophoblast cells in the maternal compartment was analyzed using the Live/Dead Cell Viability Kit (Life Technologies) following the manufacturer's instructions. Briefly, the cells in the device were washed with PBS and stained with PBS containing 4 μM ethidium homodimer and 2 μM calcein AM at 37° C. for 30 min. Subsequently, the cells were rinsed with fresh PBS and imaged using a laser scanning confocal microscope (LSM 800, Carl Zeiss, Jena, Germany). To measure LDH release from the engineered placental tissues, perfusate from the device was collected from the outlet access ports and analyzed by the Cytotoxicity Detection KitPLUS assay (LDH, Roche, 04744926001) using the manufacturer-provided protocol. Analysis of caspase-3/7 was performed by using the Caspase-Glo® 3/7 Assay (Promega). An equal volume of reagent was added and gently mixed at 37° C. for 2 hours according to the manufacture's protocol. Luminescence generated by apoptotic cells was detected and quantified using a fluorescence plate reader (Infinite M PLEX, Tecan, Männedorf, Switzerland).

### Analysis of Secretory Products

To measure β-hCG production by trophoblast cells in our model, effluent from the maternal chamber was collected on day 10 of culture. The collected media samples were then analyzed by the human β-hCG ELISA Kit (DKO014, DiaMetra) following the manufacturer-provided protocol to measure the concentration of β-hCG. For analysis of pro-inflammatory cytokines, device effluent collected from the maternal and fetal chambers was measured using the following ELISA kits: Human IL-1β ELISA Kit (RAB0273, Sigma Aldrich), Human IL-6 ELISA Kit (RAB0306, Sigma Aldrich), Human IL-8 ELISA Kit (RAB0319, Sigma Aldrich), Human TNF Alpha ELISA Kit (ab181421, abcam), and Human TGF Beta 1 ELISA Kit (DY240, R&D Systems).

### Measurement of Barrier Function

The structural integrity of the engineered placental barrier was assessed by measuring electrical resistance between the maternal and fetal chambers. Briefly, Ag/AgCl electrodes (0.008″ diameter, A-M Systems, WA, USA) connected to a multimeter (Fluke, USA) were inserted into the access ports of the maternal and fetal chambers. Resistance evaluated from an empty device without any cells was subtracted from each measurement to calculate net resistance, which was then multiplied by the surface area of the culture chamber to compute the final values expressed in Ω×cm².

Barrier permeability was evaluated by measuring transfer of tracer dyes including Lucifer Yellow CH (Invitrogen, L453) and Bovine Serum Albumin 594 conjugated (BSA, Biotium, cat #20290) from the maternal to fetal compartments. Briefly, a solution containing 1 mM Lucifer Yellow in HBSS/HEPES was introduced into the maternal chamber, while the fetal chamber was filled with HBSS/HEPES. Over a period of 2 hours, outflow from the fetal chamber was collected, and its fluorescent intensity was measured using a microplate reader (Tecan, excitation 485 nm, emission 530 nm). Please note that the pore size of the intervening membrane is an important consideration for barrier permeability—pore sizes that are smaller than 1 μm may yield excessively low barrier permeability when the fetal chamber beneath the membrane is filled with cell-laden hydrogel and the upper surface of the membrane is covered with a confluent monolayer of trophoblasts.

### RT-PCR Analysis

For RNA isolation from the maternal compartment, trophoblast cells were treated with trypsin at 37° C. for 5-10 minutes and then harvested from the device. Total RNA for PCR analysis was extracted using the High Pure RNA Isolation Kit (Roche) following the manufacturer's instructions. In the fetal compartment, the fibrin gel was dissolved with an enzyme solution mixed with trypsin. Subsequently, total RNA was isolated using the RNeasy Plant Mini Kit (Qiagen) according to the manufacturer's protocol. The RNA was then converted to cDNA using the Transcriptor First Strand cDNA Synthesis Kit (Roche). Quantitative PCR reactions were set up with the FastStart Universal SYBR Green Master (Roche) using the primers listed in Table 4. The relative expression level of each gene was calculated using the comparative threshold cycle (ΔCt) method using GAPDH as a housekeeping gene.

### Analysis of Glucose Transport

To measure maternal-to-fetal transfer of glucose in our model, the maternal chamber was perfused with trophoblast media supplemented with D-glucose (Gibco) to generate a final glucose concentration of 450 mg/dl. Culture media flowing through the fetal compartment contained 100 mg/dl of glucose. Over a 2-hour period, outflow from both the maternal and fetal chambers was collected, and its glucose concentration was measured using a digital glucose meter (Auvon, USA). The rate of maternal-to-fetal glucose transfer was evaluated by calculating the percent increase in fetal glucose concentration over the perfusion period.

### Generation of BCRP Knock-Down Model

The human choriocarcinoma BeWo cells were cultured in DMEM/F12-K (Life Technologies) supplemented with 10% fetal bovine serum and 1% penicillin-streptomycin in an incubator at 5% CO₂ and 37° C. BeWo knock-down cells were generated using BCRP lentiviral shRNA particles (sc-41151-V, Santa Cruz Biotechnology, Dallas, TX) as previously described¹⁰³. For device seeding, the sterilized membrane surface in the maternal chamber was coated with gelatin (0.1% Gelatin Solution) and fibronectin (20 μg/ml) for 2 hours and seeded with BeWo cells at 2.5×10⁶ cells/ml, after which the device was incubated at 37° C. for at least 1.5 hours to allow the cells to adhere to the membrane surface. Once cell attachment was confirmed, the chamber was connected to a pump for continuous perfusion at a flow rate of 100 μl/h. To induce syncytialization, the BeWo cells in the maternal chamber were perfused with DMEM/F12-K medium containing 50×10⁻⁶ M forskolin (Sigma).

### Plasmonic Analysis of Cadmium

Gold nanoparticles (AuNPs) with a diameter of 44 nm were synthesized using a standard sodium citrate reduction method¹⁰⁴. The AuNPs were modified with poly (ethylene glycol) (PEG) following a previously described protocol with some modifications¹⁰⁵. Briefly, 5 ml of the as-prepared AuNPs solution was stirred vigorously at room temperature. Next, 50 μl of poly(ethylene glycol) 2-mercaptoethyl ether acetic acid (Mn 2,100, Sigma Aldrich) was added dropwise, and the solution was stirred overnight. The solution was then sonicated for 3 minutes and centrifuged at 2,800 rcf for 10 minutes, twice. Subsequently, 700 μl of the PEG-modified AuNP solution was mixed with 300 μl of the medium and left for 3 hours prior to absorbance measurement. The absorbance spectra were measured using either a Cary 60 UV-vis spectrophotometer or a Cary 5000 UV-vis spectrophotometer (Agilent Technologies Inc.).

### Introduction of Human Peripheral Blood Neutrophils

Human peripheral blood neutrophils were isolated from the whole blood provided by a healthy donor using a the EasySep™ Direct Human Neutrophil Isolation Kit (StemCell Technologies, Cat #19257). To introduce the neutrophils into our exposure model, we stained the cells with Hoechst and suspended them in EGM-2 medium at the final concentration of 2.5-3×10⁶ cells/ml. The cells were then injected into the vessels through the inlet access port for one of the sides microchannels and allowed to flow through the vasculature for 24 hours while the device was maintained in a cell culture incubator. At the completion of perfusion, the device was washed with DPBS three times and imaged to quantify the number of adherent neutrophils.

### Supernatant Metabolite Extraction for Metabolomics Analysis

Samples of culture media were collected from individual microdevices at defined time points and stored at −80° C. 5 μl of media was added to 120 μl of −20° C. 25:25:10 (v/v/v) acetonitrile: methanol:water solution, vortexed for 10 seconds, and put on ice for at least 5 minutes. The resulting extract was centrifuged at 16,000×g for 20 minutes at 4° C., and the supernatant was transferred to tubes for LC-MS analysis. A procedure blank sample was generated identically without culture media, which was used later to remove the background ions.

### Metabolite Measurement by LC-MS

Metabolites were analyzed using a Vanquish Horizon UHPLC System (Thermo Scientific) coupled to an Orbitrap Exploris 480 Mass Spectrometer (Thermo Scientific). Waters XBridge BEH Amide XP Column (particle size, 2.5 μm; 150 mm (length)× 2.1 mm (i.d.)) was used for hydrophilic interaction chromatography (HILIC) separation. Column temperature was kept at 25° C. Mobile phases A=20 mM ammonium acetate and 22.5 mM ammonium hydroxide in 95:5 (v/v) water:acetonitrile (pH 9.45) and B=100% acetonitrile were used for both ESI positive and negative modes. The linear gradient eluted from 90% B (0.0-2.0 min), 90% B to 75% B (2.0-3.0 min), 75% B (3.0-7.0 min), 75% B to 70% B (7.0-8.0 min), 70% B (8.0-9.0 min), 70% B to 50% B (9.0-10.0 min), 50% B (10.0-12.0 min), 50% B to 25% B (12.0-13.0 min), 25% B (13.0-14.0 min), 25% B to 0.5% B (14.0-16.0 min), 0.5% B (16.0-20.5 min), then stayed at 90% B for 4.5 min. The flow rate was 0.15 mL/min. The sample injection volume was 5 μL. ESI source parameters were set as follows: spray voltage, 3200 V or −2800 V, in positive or negative modes, respectively; sheath gas, 35 arb; aux gas, 10 arb; sweep gas, 0.5 arb; ion transfer tube temperature, 300° C.; vaporizer temperature, 35° C. LC-MS data acquisition was operated under full scan polarity switching mode for all samples. The full scan was set as: orbitrap resolution, 120,000 at m/z 200; AGC target, 1e7; maximum injection time, 200 ms; scan range, 60-1000 m/z.

### Analysis of Metabolomics Data

LC-MS raw data files (.raw) were converted to mzXML format using ProteoWizard (version 3.0.20315). El-MAVEN (version 0.12.0) was used to generate a peak table containing m/z, retention time, and intensity for the peaks. Parameters for peak picking were the defaults except for the following: mass domain resolution, 5 ppm; time domain resolution, 10 scans; minimum intensity, 10,000; and minimum peak width, 5 scans. The resulting peak table was exported as a .csv file. Peak annotation of untargeted metabolomics data was performed using NetID with default parameters. Statistical analyses were performed using MetaboAnalyst 5.0 and 6.0.

### Statistical Analysis and Reproducibility

A minimum of three biological replicates were used for each experimental group. All data in the paper are represented as mean±standard deviation (SD). Data were analyzed with Student's t-test and with one-way and two-way ANOVA followed by Tukey's post-hoc test for multigroup pairwise comparisons. Differences were considered statistically significant at p-values <0.05. *P≤0.05, **P≤0.01, ***P≤0.001, ****P≤0.0001. GraphPad Prism (ver. 9; GraphPad Software) was used for statistical analysis.

TABLE 1
Selected maternal metabolites and their association with
human placental toxicity of environmental metals.

| | | | | | Association | |
|---|---|---|---|---|---|---|
| | | | | | with | |
| | | | | | placental | |
| | | | | | toxicity of | |
| | | | KEGG | | environmental | |
| No. | Metabolite | Type | Entry | Change | metals | Toxicant |
| 1 | Uridine | Nucleic acids | C00295 | Upregulated | Known | Arsenic |
| 2 | Uracil | Nucleic acids | C00106 | Downregulated | Known | Polychlorinated |
| | | | | | | Biphenyls |
| 3 | L-Glutamic acid | Amino acids | C00025 | Upregulated | Known | Cadmium, mercury, |
| | | | | | | cobalt, copper, |
| | | | | | | thallium, and |
| | | | | | | vanadium |
| 4 | Methylmalonic | Lipids | C02170 | Upregulated | Unknown | — |
| | acid | | | | | |
| 5 | L-Aspartic acid | Amino acids | C00049 | Upregulated | Known | Tobacco (contains |
| | | | | | | various metals) |
| 6 | Guanosine | Nucleic acids | C00387 | Upregulated | Unknown | — |
| 7 | Adenine | Nucleic acids | C00147 | Upregulated | Known | Cadmium |
| 8 | Pentadecanal | Lipids | C01948 | Downregulated | Unknown | — |
| 9 | Oxoglutaric acid | Organic acids | C00026 | Upregulated | Unknown | — |
| 10 | L-Asparagine | Amino acids | C00152 | Upregulated | Known | Tobacco (contains |
| | | | | | | various metals) |
| 11 | Erucic acid | Lipids | C08316 | Upregulated | Unknown | — |
| 12 | Nervonic acid | Lipids | C08323 | Upregulated | Unknown | — |
| 13 | L-Lactic acid | Organic acids | C00186 | Downregulated | Known | Tobacco (contains |
| | | | | | | various metals) |
| 14 | Thymidine | Nucleic acids | C00214 | Upregulated | Unknown | — |
| 15 | Arachidonic acid | Lipids | C00219 | Upregulated | Known | Particulate matter |
| | | | | | | (PM2.5) (may |
| | | | | | | contain metals) |
| 16 | Eicosadienoic | Lipids | C16525 | Upregulated | Unknown | — |
| | acid | | | | | |
| 17 | Pantothenic acid | Vitamins and | C00864 | Upregulated | Known | Cadmium |
| | | cofactors | | | | |
| 18 | Eicosapentaenoic | Lipids | C06428 | Upregulated | Known | Tobacco (contains |
| | acid | | | | | various metals) and |
| | | | | | | articulate matter |
| | | | | | | (PM2.5) (may |
| | | | | | | contain metals) |
| 19 | Docosapentaenoic | Lipids | C16513 | Upregulated | Unknown | — |
| | acid (22n-6) | | | | | |
| 20 | Folic acid | Vitamins and | C00504 | Downregulated | Unknown | — |
| | | cofactors | | | | |
| 21 | Adrenic acid | Lipids | C16527 | Upregulated | Known | Cadmium, chromium, |
| | | | | | | manganese, nickel, |
| | | | | | | copper, zinc, |
| | | | | | | mercury, and lead |
| 22 | Dihomo-gamma- | Lipids | C03242 | Upregulated | Unknown | — |
| | linolenic acid | | | | | |
| 23 | 13-OxoODE | Lipids | C14765 | Downregulated | Known | Particulate matter |
| | | | | | | (PM2.5) (may |
| | | | | | | contain metals) |
| 24 | D-Xylose | Carbohydrates | C00181 | Upregulated | Unknown | — |
| 25 | cis-Aconitic acid | Organic acids | C00417 | Downregulated | Unknown | — |
| 26 | Nandrolone | Lipids | C07254 | Downregulated | Unknown | — |
| 27 | Oxalic acid | Organic acids | C00209 | Downregulated | Unknown | — |
| 28 | Docosatrienoic | Lipids | C16534 | Upregulated | Unknown | — |
| | acid | | | | | |
| 29 | Deoxyuridine | Nucleic acids | C00526 | Upregulated | Unknown | — |
| 30 | Tiglic acid | Lipids | C08279 | Upregulated | Unknown | — |
| 31 | D-Glucose | Carbohydrates | C00031 | Upregulated | Unknown | — |
| 32 | L-Serine | Peptides | C00065 | Upregulated | Known | Tobacco (contains |
| | | | | | | various metals) |
| 33 | (R)-Lipoic acid | Lipids | C16241 | Upregulated | Unknown | — |
| 34 | N-(1-Deoxy-1- | Peptides | C00082 | Downregulated | Known | Cadmium and |
| | fructosyl)tyrosine | | | | | tobacco (contains |
| | | | | | | various metals) |
| 35 | 2-Hydroxybutyric | Lipids | C05984 | Downregulated | Unknown | — |
| | acid | | | | | |
| 36 | 3-Sulfinoalanine | Peptides | C00606 | Downregulated | Unknown | — |
| 37 | Glucosamine | Carbohydrates | C00329 | Upregulated | Known | Polychlorinated |
| | | | | | | Biphenyls and |
| | | | | | | particulate matter |
| | | | | | | (PM2.5) (may |
| | | | | | | contain metals) |
| 38 | Cyclohexaneund | Lipids | C12100 | Upregulated | Unknown | — |
| | ecanoic acid | | | | | |
| 39 | L-Alanine | Peptides | C00041 | Downregulated | Unknown | — |
| 40 | L-Phenylalanine | Peptides | C00079 | Downregulated | Unknown | — |
| 41 | Epinephrine | Hormones | C00547 | Upregulated | Unknown | — |
| | | and | | | | |
| | | transmitters | | | | |
| 42 | Carnosine | Peptides | C00386 | Upregulated | Unknown | — |
| 43 | Oleic acid | Lipids | C00712 | Upregulated | Unknown | — |
| 44 | Sedoheptulose | Carbohydrates | C02076 | Upregulated | Known | Polychlorinated |
| | | | | | | Biphenyls |
| 45 | Melibiose | Carbohydrates | C05402 | Upregulated | Unknown | — |
| 46 | Penicillin G | Antibiotics | C05551 | Downregulated | Unknown | — |
| 47 | Trichloroacetic | Peptides | C11150 | Downregulated | Unknown | — |
| | acid | | | | | |
| 48 | 3-Hydroxybutyric | Organic acids | C01089 | Downregulated | Known | Cadmium |
| | acid | | | | | |
| 49 | L-Proline | Peptides | C00148 | Downregulated | Known | Cadmium |
| 50 | Docosahexaenoic | Lipids | C06429 | Upregulated | Unknown | — |
| | acid | | | | | |
| 51 | Valerenic acid | Lipids | C09743 | Downregulated | Unknown | — |
| 52 | 17beta-Estradiol | Lipids | C09743 | Downregulated | Unknown | — |
| | 3-sulfate | | | | | |

TABLE 2
Selected fetal metabolites and their association with
human placental toxicity of environmental metals.

| | | | | | Association | |
|---|---|---|---|---|---|---|
| | | | | | with placental | |
| | | | | | toxicity of | |
| | | | KEGG | | environmental | |
| No. | Metabolite | Type | Entry | Change | metals | Toxicant |
| 1 | D-Fructose | Carbohydrates | C00095 | Upregulated | Unknown | — |
| 2 | Sebacic acid | Lipids | C08277 | Upregulated | Unknown | — |
| 3 | Edetic Acid | Vitamins and | C00284 | Downregulated | Unknown | — |
| | | cofactors | | | | |
| 4 | Adenine | Nucleic acids | C00147 | Upregulated | Known | Cadmium |
| 5 | 2-Hydroxyadipic | Lipids | C02360 | Upregulated | Unknown | — |
| | acid | | | | | |
| 6 | Eicosadienoic | Lipids | C16525 | Downregulated | Unknown | — |
| | acid | | | | | |
| 7 | Arachidonic acid | Lipids | C00219 | Downregulated | Known | Particulate matter |
| | | | | | | (PM2.5) (may |
| | | | | | | contain metals) |
| 8 | Cyclohexaneunde | Lipids | C12100 | Downregulated | Unknown | — |
| | canoic acid | | | | | |
| 9 | Dihomo-gamma- | Lipids | C03242 | Downregulated | Unknown | — |
| | linolenic acid | | | | | |
| 10 | Guanosine | Nucleic acids | C00387 | Upregulated | Unknown | — |
| 11 | Uridine | Nucleic acids | C00299 | Upregulated | Known | Arsenic |
| 12 | Thymidine | Nucleic acids | C00214 | Upregulated | Unknown | — |
| 13 | Citraconic acid | Lipids | C02226 | Upregulated | Known | Polychlorinated |
| | | | | | | Biphenyls |
| 14 | L-Glutamic acid | Peptides | C00025 | Downregulated | Known | Cadmium, |
| | | | | | | mercury, cobalt, |
| | | | | | | copper, thallium, |
| | | | | | | and vanadium |
| | | | | | | and tobacco |
| | | | | | | (contains |
| | | | | | | various metals) |
| 15 | Oleic acid | Lipids | C00712 | Downregulated | Unknown | — |
| 16 | Linoleic acid | Lipids | C01595 | Downregulated | Known | Cadmium |
| 17 | Docosahexaenoic | Lipids | C06429 | Downregulated | Unknown | — |
| | acid | | | | | |
| 18 | N- | Carbohydrates | C00270 | Upregulated | Unknown | — |
| | Acetylneuraminic | | | | | |
| | acid | | | | | |
| 19 | Pyruvic acid | Lipids | C00022 | Upregulated | Known | Inorganic arsenic |
| | | | | | | (iAs) |
| 20 | Docosatrienoic | Lipids | C16534 | Downregulated | Unknown | — |
| | acid | | | | | |
| 21 | alpha-Linolenic | Lipids | C06427 | Downregulated | Unknown | — |
| | acid | | | | | |
| 22 | 2-Hydroxybutyric | Lipids | C05984 | Upregulated | Unknown | — |
| | acid | | | | | |
| 23 | Adrenic acid | Lipids | C16527 | Downregulated | Known | Cadmium, |
| | | | | | | chromium, |
| | | | | | | manganese, |
| | | | | | | nickel, copper, |
| | | | | | | zinc, mercury, |
| | | | | | | and lead |
| 24 | D-Sorbitol | Carbohydrates | C00794 | Upregulated | Unknown | — |
| 25 | Prostaglandin F2a | Lipids | C00639 | Downregulated | Unknown | — |
| 26 | Glycerophosphoi | Lipids | C03819 | Downregulated | Known | Tobacco |
| | nositol | | | | | (contains various |
| | | | | | | metals) |
| 27 | L-Aspartic acid | Peptides | C00049 | Downregulated | Known | Tobacco |
| | | | | | | (contains various |
| | | | | | | metals) |
| 28 | Deoxyuridine | Nucleic acids | C00526 | Upregulated | Unknown | — |
| 29 | 13-OxoODE | Lipids | C14765 | Downregulated | Known | Particulate matter |
| | | | | | | (PM2.5) (may |
| | | | | | | contain metals) |
| 30 | Malic acid | Organic acids | C00149 | Downregulated | Unknown | — |
| 31 | 3-Hydroxybutyric | Lipids | C01089 | Downregulated | Known | Cadmium |
| | acid | | | | | |
| 32 | L-Lactic acid | Organic acids | C00186 | Upregulated | Known | Tobacco |
| | | | | | | (contains various |
| | | | | | | metals) |
| 33 | N-Formyl-L- | Peptides | C03145 | Downregulated | Known | Arsenic, tobacco |
| | methionine | | | | | (contains various |
| | | | | | | metals) and |
| | | | | | | inorganic arsenic |
| | | | | | | (iAs) |
| 34 | Oxoadipic acid | Lipids | C00322 | Upregulated | Unknown | — |
| 35 | L-Asparagine | Peptides | C00152 | Upregulated | Known | Tobacco |
| | | | | | | (contains various |
| | | | | | | metals) |
| 36 | Leukotriene | Lipids | C00909 | | Known | Tobacco |
| | | | | | | (contains various |
| | | | | | | metals) |
| 37 | Oxoglutaric acid | Organic acids | C00026 | Downregulated | Unknown | — |
| 38 | Norepinephrine | Hormones | C00547 | Upregulated | Known | Polychlorinated |
| | | and | | | | Biphenyls |
| | | transmitters | | | | |
| 39 | Pyroglutamic acid | Peptides | C01879 | Upregulated | Unknown | — |
| 40 | L-Homoserine | Peptides | C00263 | Downregulated | Known | Tobacco |
| | | | | | | (contains various |
| | | | | | | metals) |
| 41 | D-Xylose | Carbohydrates | C00181 | Upregulated | Unknown | — |
| 42 | Guanine | Nucleic acids | C00242 | Downregulated | Known | Cadmium |
| 43 | Citric acid | Organic acids | C00158 | Upregulated | Known | Phthalates and |
| | | | | | | metals |
| 44 | Medroxyprogester | Lipids | C07119 | Downregulated | Unknown | — |
| | one | | | | | |
| 45 | D-Glucose | Carbohydrates | C00031 | Upregulated | Unknown | — |
| 46 | Pyridoxal | Vitamins & | C00250 | Upregulated | Unknown | — |
| | | cofactors | | | | |
| 47 | L-Alanine | Peptides | C00041 | Downregulated | Unknown | — |
| 48 | L-Glutamine | Peptides | C00064 | Upregulated | Known | Tobacco |
| | | | | | | (contains various |
| | | | | | | metals) |
| 50 | L-Serine | Peptides | C00065 | Upregulated | Known | Tobacco |
| | | | | | | (contains various |
| | | | | | | metals) |
| 51 | Taurodeoxycholic | Lipids | C05463 | Downregulated | Known | Persistent organic |
| | acid | | | | | pollutants (POPs) |
| 52 | L-Dopa | Peptides | C00355 | Downregulated | Unknown | — |
| 53 | L-Tryptophan | Peptides | C00078 | Upregulated | Known | Tobacco |
| | | | | | | (contains various |
| | | | | | | metals) |
| 54 | gamma- | Lipids | C01770 | Downregulated | Unknown | — |
| | Butyrolactone | | | | | |
| 55 | L-Lysine | Peptides | C00047 | Upregulated | Known | Cadmium, cobalt, |
| | | | | | | copper, cesium, |
| | | | | | | manganese, |
| | | | | | | thallium and |
| | | | | | | vanadium |
| 56 | Mevalonic acid | Lipids | C00418 | Upregulated | Unknown | — |
| 57 | Niacinamide | Vitamins and | C00153 | Upregulated | Known | Particulate matter |
| | | cofactors | | | | (PM2.5) (may |
| | | | | | | contain metals) |
| 58 | Pantothenic acid | Vitamins and | C00864 | Upregulated | Known | Cadmium and |
| | | cofactors | | | | zinc |
| 59 | Glycerol | Carbohydrates | C00116 | Downregulated | Known | Inorganic arsenic |
| | | | | | | (iAs) |
| 60 | D-Galacturonic | Carbohydrates | C00333 | Upregulated | Known | Cadmium and |
| | acid | | | | | particulate matter |
| | | | | | | (PM2.5) (may |
| | | | | | | contain metals) |
| 61 | L-Arginine | Peptides | C00062 | Upregulated | Known | Tobacco |
| | | | | | | (contains various |
| | | | | | | metals) |

TABLE 3
Resources

| | | |
|---|---|---|
| Reagent or resource | Source | Identifier |

Antibodies

| | | |
|---|---|---|
| Alexa Fluor 488 Anti-CD31 | abcam | ab215911 |
| Anti-E-Cadherin | abcam | ab40772 |
| Anti-E-Cadherin | abcam | ab1416 |
| Anti-Glucose Transporter (GLUT1) | abcam | ab115730 |
| Anti-CD163 | abcam | ab182422 |
| Anti-CD163 | Invitrogen | MA5-17716 |
| Anti-ZO-1 | Invitrogen | 61-7300 |
| Anti-ZO-1 | Invitrogen | 33-9100 |
| Alexa Fluor 488 Anti-Fibronectin | Invitrogen | 53-9869-82 |
| Anti-alpha -Smooth Muscle | Invitrogen | 14-9760-82 |
| Anti-alpha -Smooth Muscle | R&D Systems | MAB1420 |
| Anti-BCRP | Santa Cruz | Sc-18841 |
| Alexa Fluor ™ 488 Phalloidin | Thermofisher | A-12379 |
| Alexa Fluor ™ 647 Phalloidin | Thermofisher | A-22287 |
| Donkey anti-mouse Secondary Antibody | Invitrogen | A-32773 |
| Alexa Fluor ™ Plus 555 | | |
| Goat anti-mouse Secondary Antibody | Invitrogen | A-32723 |
| Alexa Fluor ™ Plus 488 | | |
| Goat anti-mouse Secondary Antibody | Invitrogen | A-32728 |
| Alexa Fluor ™ Plus 647 | | |
| Goat anti-rabbit Secondary Antibody | Invitrogen | A-32732 |
| Alexa Fluor ™ Plus 555 | | |
| Donkey anti-rabbit Secondary Antibody | Invitrogen | A-32790 |
| Alexa Fluor ™ 488 | | |

Chemicals, peptides, and recombinant proteins

| | | |
|---|---|---|
| DMEM/F-12, HEPES, no phenol red | Gibco/Invitrogen | Catalog # 11039021 |
| EGM ™-2 MV BulletKit ™ | Lonza | Catalog # CC-3202 |
| EGM ™-2 BulletKit ™ | Lonza | Catalog # CC-3162 |
| FGM ™-2 BulletKit ™ | Lonza | Catalog # CC-3132 |
| CTBPRO2GRO ™ trophoblast media | Amnion Foundation | Catalog # CTBPRO2GRO |
| Invitrogen ™ Lucifer Yellow CH, Lithium Salt, 25 mg | Invitrogen | Catalog # L453 |
| Cadmium chloride solution | Sigma Aldrich | Catalog # 21115 |
| EasySep ™ Magnet | STEMCELL Technologies | Catalog # 18000 |
| EasySep ™ Direct Human Neutrophil Isolation Kit | STEMCELL Technologies | Catalog # 100-0404 |

Assays

| | | |
|---|---|---|
| Human IL-6 ELISA kit | Sigma Aldrich | RAB0306 |
| Human IL-8/CXCL8 ELISA Kit | Sigma Aldrich | RAB0319 |
| Human TNF alpha ELISA Kit | abcam | Ab181421 |
| Human IL-1β ELISA Kit | Sigma Aldrich | RAB0273 |
| Human TGF-beta 1 DueSet ELISA | R&D Systems | DY240 |
| β-HCG ELISA | DiaMetra | DKO014 |
| Cytotoxicity Detection Kit^{PLUS} (LDH) | Roche | 04744926001 |

Cells

| Human Cytotrophoblast Cells | Amnion Foundation | Cat #1230 |
|---|---|---|
| Placental Stromal Cells/Fibroblasts | Amnion Foundation | Cat# 1250 |
| Germinal-Origin Microvascular Endothelial Cells | Amnion Foundation | Cat #1245 |
| Hofbauer Cells | Amnion Foundation | Cat #1220 |
| BeWo-b30 human choriocarcinoma cells | Donated by Dr. Lauren | |
| | M. Aleksunes at Rutgers | |
| | University | |
| BCRP knockdown and controls | Donated by Dr. Nick Illsley | |
| Oligonucleotides | | |
| Primers for RT PCR | This disclosure | see Table 4 |

TABLE 4
List of primers

| | | SEQ ID |
|---|---|---|
| Gene | Forward Primers | No. |
| GAPDH | CGCTCTCTGCTCCTCCTGTT | 1 |
| Syn-1 | GCAACCACGAACGGACATC | 2 |
| Syn-2 | CGGATACCTTCCCTAGTGCC | 3 |
| hCG-α | CAGAATGCACGCTACAGGAA | 4 |
| hCG-β | GCACCAAGGATGGAGATGTT | 5 |
| ABCG2 | GGATGAGCCTACAACTGGCTT | 6 |
| ABCB1 | GTGGTGGGAACTTTGGCTG | 7 |
| ABCB4 | ATCGAGACGTTACCCCACAA | 8 |
| ABCC1 | GTGTTTCTGGTCAGCCCAACT | 9 |
| ABCC2 | TCCAACTGTGCTTCAAGC | 10 |
| SLC2A1 | GATGATGCGGGAGAAGAAGGT | 11 |
| SLC2A3 | CTTCCCCTCCGCTGCTCACTA | 12 |
| SLC11A1 | TGCATCTTGCTGAAGTATGTCACC | 13 |
| Twist1 | TCTCGGTCTGGAGGATGGA | 14 |
| PIGF | GGCTGTTCCCTTGCTTCCT | 15 |
| SDC1 | GGATGACTCTGACAACTTCTCC | 16 |
| ZIP8 | CAGTGTGGTATCTCTACAGGATGGA | 17 |
| ZIP14 | CAAGTCTGCAGTGGTGTITG | 18 |
| E-cad | GCCGAGAGCTACACGTTCAC | 19 |
| ZO-1 | CAACATACAGTGACGCTTCACA | 20 |
| Snail | AAGATGCACATCCGAAGCCA | 21 |
| Zeb1 | TGCACTGAGTGTGGAAAAGC | 22 |
| Zeb2 | CGCTTGACATCACTGAAGGA | 23 |
| P-53 | CATGAGCGCTGCTCAGATAG | 24 |
| MT1A | CTTGGGATCTCCAACCTCAC | 25 |
| MT2A | CCGACTCTAGCCGCCTCTT | 26 |
| ZnT1 | CCTGGGCTTCTTCTCTAGATTG | 27 |
| ZnT2 | CCTGGTCTCTGTACTGTCCATCT | 28 |
| SOD | GCAGAAGGCAAGCGGTGAAC | 29 |
| CAT | GCGAATGGAGAGGCAGTGTAC | 30 |
| GPX1 | AGATGTCATTCCTGCACACG | 31 |
| BCL-2 | TCCCTCGCTGCACAAATACTC | 32 |
| Nrf2 | CGCAGACATTCCCGTTTGTAGA | 33 |
| NF-κB | GCAAAGGGAACATTCCGATAT | 34 |
| TRPV2 | TGTAGCCCTGGTGAGCCT | 35 |
| TRPV4 | CTACGGCACCTATCGTCACC | 36 |
| Caspase3 | AATTGTGGAATTGATGCGTGATG | 37 |
| Caspase7 | CCAATAAAGGATTTGACAGCC | 38 |
| Caspase9 | ATGGACGAAGCGGATCGG | 39 |
| VEGFR1 | CAGGCCCAGTTTCTGCCATT | 40 |
| VEGFR2 | CCAGCAAAAGCAGGGAGTCTGT | 41 |
| PECAM1 | GAGTATTACTGCACAGCCTTCA | 42 |
| CLDN5 | GTTCGCCAACATTGTCGTCC | 43 |
| VCAM-1 | CCGTCTCATTGACTTGCAGC | 44 |
| COL1A1 | ATCAACCGGAGGAATTTCCGT | 45 |
| COL1A2 | GGCCCTCAAGGTTTCCAAGG | 46 |
| FN1 | CAGGATCACTTACGGAGAAACAG | 47 |
| CTGF | GCAGGCTAGAGAAGCAGAGC | 48 |
| PDGF | TGATCTCCAACGCCTGCT | 49 |
| ICAM-1 | AACCAGAGCCAGGAGACACT | 50 |
| MMP-2 | ACATCAAGGGCATTCAGGAG | 51 |
| MMP-9 | GGGAAGATGCTGCTGTTCA | 52 |
| TIMP-1 | TCAACCAGACCACCTTATACCA | 53 |
| | | SEQ ID |
| Gene | Reverse Primers | No. |
| GAPDH | CCATGGTGTCTGAGCGATGT | 54 |
| Syn-1 | GTATCCAAGACTCCACTCCAGC | 55 |
| Syn-2 | AGCTGAGGTTGCTGGTTCTG | 56 |
| hCG-α | CGTGTGGTTCTCCACTITGA | 57 |
| hCG-β | GCACATTGACAGCTGAGAGC | 58 |
| ABCG2 | CTTCCTGAGGCCAATAAGGTG | 59 |
| ABCB1 | TACCTGGTCATGTCTTCCTCC | 60 |
| ABCB4 | CATTCTGGATGGTGGACAGG | 61 |
| ABCC1 | TTGGATCTCAGGATGGCTAGG | 62 |
| ABCC2 | GGCATCCACAGACATCAG | 63 |
| SLC2A1 | ACAGCGTTGATGCCAGACAG | 64 |
| SLC2A3 | CAAAAGTCCTGCCACGGGTCT | 65 |
| SLC11A1 | CTCCACCATCAGCCACAGGAT | 66 |
| Twist1 | CAATGACATCTAGGTCTCCG | 67 |
| PIGF | TACCACTTCCACCTCTGACGA | 68 |
| SDC1 | CTACAGCCTCTCCCTCCTT | 69 |
| ZIP8 | CAGTTTGGGCCCCTTCAAA | 70 |
| ZIP14 | GTGTCCATGATGATGCTCATTT | 71 |
| E-cad | GTCGAGGGAAAAATAGGCTG | 72 |
| ZO-1 | CACTATTGACGTTTCCCCACTC | 73 |
| Snail | CATTCGGGAGAAGGTCCGAG | 74 |
| Zeb1 | TGGTGATGCTGAAAGAGACG | 75 |
| Zeb2 | CTTGCCACACTCTGTGCATT | 76 |
| P-53 | ACACGCAAATTTCCTTCCAC | 11 |
| MT1A | AGGAGCAGCAGCTCTTCTTG | 78 |
| MT2A | GTGGAAGTCGCGTTCTTTACA | 79 |
| ZnT1 | TTGTCTTGGAAAGGTTGTTCTG | 80 |
| ZnT2 | GATCACGAACAGCTGTGAAGTC | 81 |
| SOD | TAGCAGGACAGCAGATGAGT | 82 |
| CAT | GAGTGACGTTGTCTTCATTAGCACTG | 83 |
| GPX1 | AAGGAGAAGCTTCCTCAGCC | 84 |
| BCL-2 | TTCTGCCCCTGCCAAATCT | 85 |
| Nrf2 | GTGACCGGGAATATCAGGAACAAG | 86 |
| NF-κB | GCGACATCACATGGAAATCTA | 87 |
| TRPV2 | CCAACGGTCAGCATCACA | 88 |
| TRPV4 | CTGCGGCTGCTTCTCTATGA | 89 |
| Caspase3 | CTACAACGATCCCCTCTGAAAAA | 90 |
| Caspase7 | GCATCTGTGTCATTGATGGG | 91 |
| Caspase9 | CCCTGGCCTTATGATGTT | 92 |
| VEGFR1 | TTCCAGCTCAGCGTGGTCGTA | 93 |
| VEGFR2 | TGTCTGTGTCATCGGAGTGATATCC | 94 |
| PECAM1 | AACCACTGCAATAAGTCCTTTC | 95 |
| CLDN5 | GTAGTTCTTCTTGTCGTAGTCGC | 96 |
| VCAM-1 | GATGTGGTCCCCTCATTCGT | 97 |
| COL1A1 | CACCAGGACGACCAGGTTTTC | 98 |
| COL1A2 | CACCCTGTGGTCCAACAACTC | 99 |
| FN1 | GCCAGTGACAGCATACACAGTG | 100 |
| CTGF | ATGTCTTCATGCTGGTGCAG | 101 |
| PDGF | TCATGTTCAGGTCCAACTCG | 102 |
| ICAM-1 | GAGACCTCTGGCTTCGTCAG | 103 |
| MMP-2 | GCCTCCGTATACCGCATCAAT | 104 |
| MMP-9 | TCAACTCACTCCGGGAACTC | 105 |
| TIMP-1 | ATCCGCAGACACTCCAT | 106 |

### Aspects

Aspect 1. A placenta-on-a-chip fluidic chip, comprising: a bottom region, the bottom region comprising a central channel and at least one side channel adjacent thereto, the at least one side channel having therein a plurality of placental or placenta-derived cells, the central channel having therein a plurality of cells disposed in a matrix; and a upper region, the upper region having an upper channel defined therein, the upper channel being in register with at least one of the central channel and the at least one side channel of the bottom region.

An example chip is provided in FIG. 3C. As shown in that figure, chip 300 can include a bottom portion that includes central channel 301 (which can be configured as a chamber, in some embodiments) and side channel 303. Side channel 303 can include placental or placental-derived cells. Chip 300 can also include an upper region, which upper region can include upper channel 305 (which can be configured as a chamber, in some embodiments). As shown, upper channel 305 can be in register or at least partial register with at least one of central channel 301 and side channel 303. Central channel 301 can be referred to as a primary channel in some instances, and there is no requirement that central channel 301 be centered within any part of chip 300 or be centered within chip 300, as the term “central” is used for convenience. As shown, rail 309 can be arranged between side channel 303 and central channel 301.
Aspect 2. The fluidic chip of Aspect 1, further comprising a pervious membrane, the pervious membrane being arranged between the upper channel of the upper region and at least one of (i) the central channel of the bottom region or (ii) the at least one side channel of the bottom region. As shown in FIG. 3C, pervious medium 307 can be arranged between upper channel 305 and central channel 301.

FIG. 3Dd provides a depiction of a chip 300a according to the present disclosure. As shown, chip 300a can include a cap 311. Cap 311 can include an inlet ports, shown by inlet port 313, inlet port 315, and inlet port 316. Chip 300a can also include an upper portion 317; as shown, upper portion 317 can include upper chamber 321, which can also be referred to as the maternal chamber. Upper portion 317 can also include an aperture 319, which aperture can be in register with one or more inlet ports, as shown. Upper chamber 321 can be in at least partial register or fluid communication with an inlet port, such as an inlet port of cap 311. Chip 300a can also include a pervious membrane 323.

Chip 300a can further include bottom portion 325. As shown, bottom portion 325 can include a central channel 331, which can also be termed the fetal chamber in some instances. Bottom portion 325 can also include side channel 327; as shown, rail 329 can be arranged between central channel 331 and side channel 327. An inlet port-such as inlet port 316—can be in at least partial register or fluid communication with one or more of side channel 327 and central channel 331. It should be understood, however, that a chip according to the present disclosure need not be composed of different sections assembled together, as a chip according to the present disclosure can be a single, integral body that is not formed from assembling various parts together. A chip according to the present disclosure can, however, be formed of a plurality of sections assembled together.
Aspect 3. The fluidic chip of any one of Aspects 1-2, further comprising trophoblast cells disposed within the upper channel, the trophoblast cells optionally superposed on the pervious membrane, and the trophoblast cells optionally being syncytialized.
Aspect 4. The fluidic chip of any one of Aspects 1-3, wherein the matrix comprises a hydrogel.
Aspect 5. The fluidic chip of any one of Aspects 1-4, wherein the plurality of cells comprises any one or more fibroblasts and endothelial.
Aspect 6. The fluidic chip of any one of Aspects 1-5, wherein the central channel has therein a perfusable vascular network comprising endothelial cells.
Aspect 7. The fluidic chip of Aspect 6, wherein the perfusable vascular network comprises at least one vessel that places the central channel into fluid communication with the at least one side channel. One such example is provided in FIG. 3C.
Aspect 8. The fluidic chip of any one of Aspects 1-7, wherein the side channel includes an endothelium having a luminal surface, the endothelium being from or derived from the placenta.
Aspect 9. The fluidic chip of Aspect 8, wherein the luminal surface is in fluid communication with a source of fluid.
Aspect 10. The fluidic chip of any one of Aspects 1-9, wherein the upper channel is configured as a material channel and wherein the central channel is configured as a fetal channel.
Aspect 11. The fluidic chip of any one of Aspects 1-10, wherein the bottom region and the upper region are separable from one another.
Aspect 12. The fluidic chip of any one of Aspects 1-11, wherein the bottom region and the upper region comprise a polymeric material.
Aspect 13. The fluidic chip of any one of Aspects 1-12, further comprising a rail disposed between the central channel and the at least one side channel.
Aspect 14. A method, comprising communicating an agent within the upper channel of a fluidic chip according to any one of Aspects 1-12 and monitoring a response of at least one of a tissue in the upper channel and a tissue in the central channel.

Monitoring can include, without limitation, staining specific markers indicative of cell viability, maturity, physiological function, and the like. One can also collect effluent from the chip and to measure biomolecules secreted by the exposed cells and tissues, including proteins, lipids, metabolites, nucleic acids, hormones, extracellular vesicles, and the like. Further, one can also harvest cells and their surrounding matrices for RNA sequencing analysis. These tissues can also be processed for histological analysis. One can monitor a marker indicative of exposure to the agent; the marker can be indicative of any one or more of tissue damage, a secretory product, golgi damage, an oxidative stress, a barrier function, a fibrinogenic response, a cell marker expression, a morphological change, an inflammatory response, a transcriptomic response, a lipidomic response, a metabolomic response, a proteomic response, and an unfolded protein response. A marker can include any one or more of cell viability, apoptosis, necrosis, cell proliferation, DNA damage, cell sloughing, a golgi morphology or a change thereof, a golgi marker expression or a change thereof, a reactive oxygen species or a change thereof, a lipid peroxidation product or a change thereof, a protein oxidation product or a change thereof, a DNA oxidation product or a change thereof, an antioxidant secretion or a change thereof, an interleukin secretion or a change thereof, an endothelin-1 secretion or a change thereof, a secretion of mucus or a change thereof, a secretion of a surfactants or a change thereof, a barrier permeability, a TEER level or a change thereof, an impedance or a change thereof, a gene expression or a change thereof, a lipid expression or a change thereof, a protein expression or a change thereof, a metabolite level or a change thereof, a cell-specific marker expression or a change thereof, a TGF-α release or a change thereof, a TGF-β release or a change thereof, an ECM deposition or a change thereof, a PERK level, a CHOP level, an IRE1 level, an eIF2a level, and an ATF6 level. As an example, one can monitor changes in one or more molecules associated with the unfolded protein response.
Aspect 15. The method of Aspect 14, wherein the agent is a toxin. Example toxins include, without limitation, metals.

What is claimed:

1. A placenta-on-a-chip fluidic chip, comprising:
a bottom region,
the bottom region comprising a central channel and at least one side channel adjacent thereto,
the at least one side channel having therein a plurality of placental or placenta-derived cells,
the central channel having therein a plurality of cells disposed in a matrix; and
a upper region,
the upper region having an upper channel defined therein,
the upper channel being in register with at least one of the central channel and the at least one side channel of the bottom region.

2. The fluidic chip of claim 1, further comprising a pervious membrane, the pervious membrane being arranged between the upper channel of the upper region and at least one of (i) the central channel of the bottom region or (ii) the at least one side channel of the bottom region.

3. The fluidic chip of claim 1, further comprising trophoblast cells disposed within the upper channel, the trophoblast cells optionally superposed on the pervious membrane, and the trophoblast cells optionally being syncytialized.

4. The fluidic chip of claim 1, wherein the matrix comprises a hydrogel.

5. The fluidic chip of claim 1, wherein the plurality of cells comprises any one or more fibroblasts and endothelial.

6. The fluidic chip of claim 1, wherein the central channel has therein a perfusable vascular network comprising endothelial cells.

7. The fluidic chip of claim 6, wherein the perfusable vascular network comprises at least one vessel that places the central channel into fluid communication with the at least one side channel.

8. The fluidic chip of claim 1, wherein the side channel includes an endothelium having a luminal surface, the endothelium being from or derived from the placenta.

9. The fluidic chip of claim 8, wherein the luminal surface is in fluid communication with a source of fluid.

10. The fluidic chip of claim 1, wherein the upper channel is configured as a material channel and wherein the central channel is configured as a fetal channel.

11. The fluidic chip of claim 1, wherein the bottom region and the upper region are separable from one another.

12. The fluidic chip of claim 1, wherein the bottom region and the upper region comprise a polymeric material.

13. The fluidic chip of claim 1, further comprising a rail disposed between the central channel and the at least one side channel.

14. A method, comprising communicating an agent within the upper channel of a fluidic chip according to claim 1 and monitoring a response of at least one of a tissue in the upper channel and a tissue in the central channel.

15. The method of claim 14, wherein the agent is a toxin.
